(19) United States
(12) Patent Application Publication
(10) Pub. No.: US 2004/0078844 A1
(43) Pub. Date: Apr. 22, 2004

(54) Methods and means for gene silencing

(76) Inventors: David Charles Baulcombe, Norwich (GB); Ana Monserrat Martin-Hernandez, Norwich (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/362,144

(22) PCT Filed: Aug. 13, 2001
(86) PCT No.: PCT/GB01/03623

(30) Foreign Application Priority Data
Aug. 17, 2000 (GB) 0020320.8

(51) Int. Cl. A01H 1/00; C12N 15/82

(52) U.S. Cl. 800/280; 435/468

(57) ABSTRACT

Provided are insolated DNA vectors which may be based on Agrobacterium binary vectors and which comprise: (a) a transfer nucleotide sequence comprising (i) a plant active promoter, operably linked to (ii) a recombinant tobacco rattle virus (TRV) nucleic acid which may corresponds to all or part of TRV RNA 1 and which includes: a sequence encoding a TRV trans acting factor, and cis acting elements, which confer on the TRV nucleic acid transcript the ability to replicate in the cytoplasm of a plant cell; and a heterologous nucleotide sequence which is foreign to said virus (which may be a cloning site, or a targeting sequence which is capable of down-regulating expression of a target gene); (b) border sequences which permit the transfer of the transfer nucleotide sequence into a plant cell genome. Preferred vectors include pBTAΔAMPΔ16K (SEQ ID NO: 3) or pBTAΔMP (SEQ ID NO: 2). Also provided are related materials and methods of use of such vectors e.g. to produce a cytoplasmically-replicating RNA which can be used to silence target genes in plants.

### TECHNICAL FIELD

The present invention relates generally to recombinant, replicable, plant-viral based nucleic acid constructs, and methods of use thereof in silencing genes in plants.

### PRIOR ART

In plants, post-transcriptional gene silencing (PTGS) can be manifested as an inhibition of nuclear gene expression after the infection with a virus which has been modified to carry sequence from a nuclear expressed gene (Kjemtrup et al., 1998; Kumagai et al., 1995; Ruiz et al., 1998) PTGS can also be manifested after the insertion of a transgene into the plant genome (Napoli et al., 1990; van der Krol et al., 1990). In 2-20% of the cases, after the plant transformation, the plant shows the loss-of function phenotype for the inserted gene instead of its overexpression (Angell and Baulcombe, 1999 and references therein). In both cases, the loss-of function phenotype is caused by sequence specific RNA degradation.

When the transgene contains the sequence of a replicating virus carrying sequence from a nuclear expressed gene (amplicon virus), the transgenic plant shows the null phenotype for the homologous plant gene in 100% of the plants expressing the replicating amplicon (Angell and Baulcombe, 1999). This null phenotype is stable and inherited through subsequent generations (Angell and Baulcombe, 1997). Therefore, amplicon technology can be used to identify the function of any gene and at the same time, to have the actual knock-out plant for the gene whose function is being identified.

There are a number of examples of transgenic plants carrying constructs based on whole viral genomes (Atkinson et al., 1998) Kaido et al., 1995; Mori et al., 1993; Yamaya et al., 1988), but most amplicon work has been done with potato virus X (PVX)(Angell and Baulcombe, 1997; Angell and Baulcombe, 1999). In those studies, PVX amplicon was used to transform Nicotiana species and tomato, and silence a number of endogenous genes in these plants (Angell and Baulcombe, 1999). PVX amplicon plants produced infectious viruses, but without any viral symptoms overlapping the silencing phenotype.

### DISCLOSURE OF THE INVENTION

The present invention is concerned with novel viral amplicon constructs. In preferred forms the present invention is concerned with providing amplicon-based methods and materials which may be more suitable as a tool for functional genomics than those which have been used in the past.

For instance, PVX amplicon Nicotiana plants may not exhibit silencing of genes expressed in meristems (Angell and Baulcombe, 1999). Additionally, PVX has a relatively narrow spectrum of hosts suggesting that it may be difficult to produce silencing of non-host PVX amplicon plants. For example, Arabidopsis thaliana PVX amplicon plants show only weak silencing (Dalmay et al., 2000) and endogenous genes in particular may be difficult to target (Dalmay, unpublished results).

The present inventors have developed novel viral amplicon constructs based on tobacco rattle virus (TRV) which seeks to address one or more of the problems of the prior art. TRV is able to invade meristems and has a broad range of hosts, including Arabidopsis thaliana.

Certain viral expression vectors based on TRV have previously been described in which non-viral proteins were expressed from a sub-genomic promoter (Ratcliff, MacFarlane et al. 1999). In that case the viral RNA was synthesised in vitro and then inoculated into the plant. Other TRV based vectors are disclosed by Hamilton & Baulcombe (1989) J. Gen. Virol 70: 963-968 and Mueller et al (1997) J. Gen. Virol 78: 2085-2088.

Thus in a first aspect of the present invention there is disclosed a nucleic acid vector which comprises:

(a) a transfer nucleotide sequence comprising (i) a plant active promoter, operably linked to (ii) a recombinant tobacco rattle virus (TRV) nucleic acid which includes:

a sequence encoding a TRV trans acting factor, and cis acting elements, which confer on the TRV nucleic acid transcript the ability to replicate in the cytoplasm of a plant cell;

a heterologous nucleotide sequence which is foreign to said virus;

(b) border sequences which permit the transfer of the transfer nucleotide sequence into a plant cell nucleus.

The transfer nucleotide sequence is situated between the border sequences and is capable of being inserted into a plant genome under appropriate conditions. Generally this may be achieved by use of so called “agro-infiltration” which uses Agrobacterium-mediated transient transformation. Briefly, this technique is based on the property of Agrobacterium tumafaciens to transfer a portion of its DNA (“T-DNA”) into a host cell where it may become integrated into nuclear DNA. The T-DNA is defined by left and right border sequences which are around 25 nucleotides in length. In the present invention the border sequences are included around the transfer nucleotide sequence (the T-DNA) with the whole vector being introduced into the plant by agro-infiltration, optionally in the form of a binary-transformation vector.

By “plant active promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. Nucleic acid operably linked to a promoter is “under transcriptional initiation regulation” of the promoter.

TRV is a bipartite virus, whose genome is composed of two positive stranded RNAs. RNA 1 carries the genes encoding for the replicase, the movement protein (MP) and a small protein called 16K, the precise function of which is unknown. RNA 2 carries the genes for the coat protein (CP) and two proteins involved in nematode transmission (Hernandez et al., 1995).

The TRV nucleic acid of the present invention includes cis and trans acting elements permitting replication of said cDNA. The vectors of the present invention will generally not require supplementary proteins and/or nucleic acids from TRV in order to achieve this. For instance the cDNA may correspond to all or part of TRV RNA 1. In preferred forms of the invention minimal amplicon constructs are used wherein genes involved in movement of the virus (e.g. MP) and other genes (e.g. 16K), may be removed, thereby leaving only those genes involved in viral replication i.e. one or more trans factors (replicase genes) and cis factors (5′ and 3′ untranslated regions). Generally the constructs will not encode a coat protein.

The TRV replicase (as with other defined or recited sequences herein) need not be ‘wild-type’, but may optionally be a variant (e.g. mutant, or other variant, or a substantially homologous derivative) provided that its function (to permit, in conjunction with the cis-elements, replication of the TRV nucleic acid transcript) is not negated. By “Substantially homologous” is meant that the sequence in question shares at least about 70%, or 80% identity, most preferably at least about 90%, 95%, 96%, 97%, 98% or 99% identity with the reference sequence. Identity may be at the nucleotide sequence and/or encoded amino acid sequence level.

Homology may be over the full-length of the relevant sequence shown herein (e.g. in the sequence Annex) or may be over a part of it. Identity may be determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using FASTA and FASTP (see Pearson & Lipman, 1988. Methods in Enzymology 183: 63-98). Parameters are preferably set, using the default matrix, as follows:

Gapopen (penalty for the first residue in a gap): −12 for proteins/−16 for DNA; Gapext (penalty for additional residues in a gap): −2 for proteins/−4 for DNA; KTUP word length: 2 for proteins/6 for DNA.

The heterologous nucleotide sequence is foreign (non-native) to TRV, which is to say that it does not occur naturally in the TRV viral genome at the position in which it is present in the VIGS vector. The sequence will generally be either a cloning site (to permit the insertion of a desired sequence) or a desired sequence itself. It may be introduced in place of other sequence which has been removed (e.g. MP sequence) or as a fusion with all or part of that sequence.

Some preferred embodiments of the invention will now be discussed.

Vector

Nucleic acid vectors according to the present invention may be provided isolated and/or purified, in substantially pure or homogeneous form, or free or substantially free of other nucleic acid. The term “isolated” encompasses all these possibilities.

Nucleic acid according to the present invention may be polynucleotides or oligonucleotides, and may include cDNA, RNA, genomic DNA and modified nucleic acids. Where a DNA sequence is specified, e.g. with reference to a figure, unless context requires otherwise the RNA equivalent, with U substituted for T where it occurs, is encompassed.

Where a nucleic acid (or nucleotide sequence) of the invention is referred to herein, the complement of that nucleic acid (or nucleotide sequence) will also be embraced by the invention. The ‘complement’ in each case is the same length as the reference, but is 100% complementary thereto whereby by each nucleotide is base paired to its counterpart i.e. G to C, and A to T or U.

Preferably the vector is based on plant binary transformation vector pBINTRA6 (see Materials and Methods below).

Generally speaking, in the light of the present disclosure, those skilled in the art will be able to construct vectors according to the present invention. Such vectors may include, in addition to the promoter, a suitable terminator or other regulatory sequence such as to define an expression cassette consisting of the recombinant TRV nucleic acid, including the heterologous nucleotide sequence. For further details see, for example, Molecular Cloning: a Laboratory Manual: 2nd edition, Sambrook et al, 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second Edition, Ausubel et al. eds., John Wiley & Sons, 1992. Specific procedures and vectors previously used with wide success upon plants are described by Bevan, Nucl. Acids Res. (1984) 12, 8711-8721), and Guerineau and Mullineaux, (1993) Plant transformation and expression vectors. In: Plant Molecular Biology Labfax (Croy RRD ed) Oxford, BIOS Scientific Publishers, pp 121-148.

Plant Promoter

Suitable promoters will be well known to those skilled in the art and will generally either be constitutive or inducible (e.g. developmentally regulated or tissue specific). Preferred examples include the Cauliflower Mosaic Virus 35S (CaMV 35S) gene promoter that is expressed at a high level in virtually all plant tissues. The promoter may in principle be an inducible promoter such as the maize glutathione-S-transferase isoform II (GST-II-27) gene promoter which is activated in response to application of exogenous safener (WO93/01294, ICI Ltd). The GST-II-27 gene promoter has been shown to be induced by certain chemical compounds which can be applied to growing plants. Another suitable promoter may be the DEX promoter (Plant Journal (1997) 11: 605-612).

Recombinant TRV Nucleic Acid

This is preferably based on a modified, reduced, cDNA clone of TRV RNA 1. In the Examples herein the strain used is ppk20. However any appropriate strain, which can give rise to replicating, infectious viral transcripts, could be used (see e.g. Macfarlane, 1999 for further examples).

Within the cDNA it is preferable that non-essential ORFs or other sequences are deleted, provided that the CDNA can still be used to generate (cytoplasmically) replicating, infectious transcripts. Preferably, where the cDNA is based on TRV RNA1 of ppk20, one or both of the open reading frames (MP and 16K) are deleted to leave only the 5′ and 3′ untranslated regions and the viral gene encoding the replicase. One or more of the deleted ORFs may be replaced by a heterologous nucleotide sequence (positioned between the UTRs so as to ensure it is replicated).

Preferred vectors include pBTAΔMPΔ16K or pBTAΔMP. The sequences are shown in the Sequence appendixes. Naturally substantially homologous variants of the sequence are also included within the scope of the invention. In particular, vectors derived from pBTAΔMPΔ16K and having the characteristics (described herein) of that vector, are also embraced.

Heterologous Nucleotide Sequence.

This can in principle be a single or multiple cloning site (i.e. a short non-coding sequence encoding two, three or more restriction endonuclease target sites) to facilitate the incorporation of a desired nucleotide sequence.

Generally the sequence will be a “targeting sequence” which corresponds to a sequence in a target gene, either in the sense or anti-sense (complementary) orientation, or a sequence which has sufficient homology to a target sequence for down-regulation of expression of the target gene to occur. Such a targeting sequence may be included in the vector anywhere in the viral cDNA irrespective of the location of any subgenomic promoter (provided it does not interfere with the cis-acting replication elements or the coat protein). Generally speaking it may be preferable for the TRV amplicons of the present invention not to include a subgenomic promoter within or operably linked to the heterologous gene sequence. Such preferred vectors have the advantage that they are more stable (reduced likelihood of self-recombination) that those of the prior art such as those described by Ratcliff, MacFarlane et al. (1999) supra which had more than one subgenomic promoter.

In general the targeting sequence may be derived from a plant nuclear gene or transgene, or a gene on an extrachromosomal element such as a plastid.

Amplicon induced PTGS are particularly preferred for investigating gene function in that it can be used to impose an intermediate or a null phenotype for a particular gene, which can provide information about the function of that gene in vivo. In such cases the identity of the targeting gene may not be known, but the methods of the present invention may be used to identify it with a particular phenotype.

The complete sequence corresponding to the coding sequence (in reverse orientation for anti-sense) need not be used. For example fragments of sufficient length may be used. It is a routine matter for the person skilled in the art to screen fragments of various sizes and from various parts of the coding sequence to optimise the relationship between target and targeting sequence. It may be preferable that there is complete sequence identity between the targeting sequence in the vector and the target sequence in the plant, although total complementarity or similarity of sequence is not essential. One or more nucleotides may differ in the targeting sequence from the target gene. Thus, a targeting sequence employed in a construct in accordance with the present invention may be a wild-type sequence (e.g. gene) selected from those available, or a substantially homologous mutant, derivative, variant or allele, by way of insertion, addition, deletion or substitution of one or more nucleotides, of such a sequence. Such a sequence need not include an open reading frame or specify an RNA that would be translatable. A typical construct may include a sequence wherein the homology (similarity or identity) between the targeting sequence and the sequence within the target gene is greater than: 80, 85, 90 or 95%, and/or a sequence which targets at least the initiating ATG codon of the target gene.

A further possibility is to target a conserved sequence of a gene, e.g. a sequence that is characteristic of one or more genes in one or more pathogens against which resistance is desired, such as a regulatory sequence. Thus a construct may target a conserved sequence within a target gene group such as to down-regulate expression of one or more members of a target gene group. More than one targeting sequence may be included.

Target genes include those which confer ‘unwanted’ traits in the plant and which it may therefore be desired to silence using amplicon-induced PTGS. Examples include ripening specific genes in tomato to improve processing and handling characteristics of the harvested fruit; genes involved in pollen formation so that breeders can reproducibly generate male sterile plants for the production of F1 hybrids; genes involved in lignin biosynthesis to improve the quality of paper pulp made from vegetative tissue of the plant; gene silencing of genes involved in flower pigment is production to produce novel flower colours; gene silencing of genes involved in regulatory pathways controlling development or environmental responses to produce plants with novel growth habit or (for example) disease resistance; elimination of toxic secondary metabolites by gene silencing of genes required for toxin production.

Other aspects of the invention will now be discussed.

One aspect of the present invention is a process for producing a vector as described above, the process being substantially as set out in the Examples hereinafter. A further aspect is a process for producing a vector as described above, which process comprises the step of cloning a heterologous nucleotide sequence which is a targeting sequence into the vector.

A further aspect of the present invention includes a method of silencing a target gene in a plant tissue using amplicon induced PTGS which method comprises the steps of introducing a vector as described above into the plant, wherein said vector includes a heterologous nucleotide sequence which is a targeting sequence.

“Plant tissue” is any tissue of a plant in planta or in culture, including the whole plant an organ thereof, a cutting, or any group of plant cells organised into a structural and functional unit.

“Silencing” is a term generally used to refer to suppression of expression of a gene. The degree of reduction may be so as to totally abolish production of the encoded gene product, but more usually the abolition of expression is partial, with some degree of expression remaining. The term should not therefore be taken to require complete “silencing” of expression. It is used herein where convenient because those skilled in the art well understand this.

As discussed above, for introduction into the plant, the vector may be in the form of an Agrobacterium binary vector. The vector is introduced into the plant cell by Agrobacterium-mediated T-DNA transfer, the transfer sequence may be integrated transiently into the plant (cell) genome, and is then transcribed to RNA from the plant promoter. In the published vector of Ratcliff, MacFarlane et al. (1999), the viral cDNA and any cDNA inserted after the sub-genomic promoter was transcribed to infectious RNA in vitro by T7 RNA polymerase and subsequently introduced into the plant.

Transient Agrobacterium mediated expression in the plant of the vector is the preferred means of introducing the vector.

Any appropriate method of plant transformation may be used to generate plant cells containing a construct within the genome in accordance with the present invention. Following transformation, plants may be regenerated from transformed plant cells and tissue.

Successfully transformed cells and/or plants, i.e. with the construct incorporated into their genome, may be selected following introduction of the nucleic acid into plant cells, optionally followed by regeneration into a plant, e.g. using one or more marker genes such as antibiotic resistance.

Plants transformed with the DNA segment containing the sequence may be produced by standard techniques which are already known for the genetic manipulation of plants. DNA can be transformed into plant cells using any suitable technology, such as a disarmed Ti-plasmid vector carried by Agrobacterium exploiting its natural gene transfer ability (EP-A-270355, EP-A-0116718, NAR 12 (22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser—see attached) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d). Physical methods for the transformation of plant cells are reviewed in Oard, 1991, Biotech. Adv. 9: 1-11.

Agrobacterium transformation is widely used by those skilled in the art to transform dicotyledonous species. Production of stable, fertile monocot transgenic plants may be achieved e.g. using the techniques of, or analogous to, Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. (1990) Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; W092/14828). In particular, Agrobacterium mediated transformation is now emerging also as an highly efficient transformation method in monocots (Hiei et al. (1994) The Plant Journal 6, 271-282).

The generation of fertile transgenic plants has been achieved in the cereals rice, maize, wheat, oat, and barley (reviewed in Shimamoto, K. (1994) Current Opinion in Biotechnology 5, 158-162; Vasil, et al. (1992) Bio/Technology 10, 667-674; Vain et al., 1995, Biotechnology Advances 13 (4): 653-671; Vasil, 1996, Nature Biotechnology 14 page 702).

Microprojectile bombardment, electroporation and direct DNA uptake are preferred where Agrobacterium is inefficient or ineffective. Alternatively, a combination of different techniques may be employed to enhance the efficiency of the transformation process, eg bombardment with Agrobacterium coated microparticles (EP-A-486234) or microprojectile bombardment to induce wounding followed by co-cultivation with Agrobacterium (EP-A-486233).

Following transformation, a plant may be regenerated, e.g. from single cells, callus tissue or leaf discs, as is standard in the art. Almost any plant can be entirely regenerated from cells, tissues and organs of the plant. Available techniques are reviewd in Vasil et al., Cell Culture and Somatic Cel Genetics of Plants, Vol I, II and III, Laboratory Procedures and Their Applications, Academic Press, 1984, and Weissbach and Weissbach, Methods for Plant Molecular Biology, Academic Press, 1989.

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

The present invention may particularly be applied in plants which are natural hosts (compatible with) TRV. By “compatible” is meant capable of operating with the other components of a system, in this case TRV must be capable of replicating in the plant in question. These include Arabidopsis thaliana. Others include (but are not limited to) Allium cepa; Amaranthus caudatus; Amaranthus retroflexus; Antirrhinum majus; snap-dragon; Arachis hypogaea; Avena sativa; Bellis perennis; Beta vulgaris; Brassica campestris; Brassica campestris ssp. napus; Brassica campestris ssp. pekinensis; Brassica juncea; Calendula officinalis; Capsella bursa-pastoris; Capsicum annuum; Catharanthus roseus; Cheiranthus cheiri; Chenopodium album; Chenopodium amaranticolor; Chenopodium foetidum; Chenopodium quinoa; Coriandrum sativum; Cucumis melo; Cucumis sativus; Glycine max; Gomphrena globosa; Gypsophila elegans; Helianthus annuus; Hyacinthus; Hyoscyamus niger; Lactuca sativa; Lathyrus odoratus; Linum usitatissimum; Lobelia erinus; Lupinus mutabilis; Lycopersicon esculentum; Lycopersicon pimpinellifolium; Melilotus albus; Momordica balsamina; Myosotis sylvatica; Narcissus pseudonarcissus; Nicandra physalodes; Nicotiana benthamiana; Nicotiana clevelandii; Nicotiana glutinosa; Nicotiana rustica; Nicotiana sylvestris; Nicotiana tabacum; Nicotiana edwardsonii; Ocimum basilicum; Petunia hybrida; Phaseolus vulgaris; Phytolacca americana; Pisum sativum; Raphanus sativus; Ricinus communis; Salvia splendens; Senecio vulgaris; Solanum melongena; Solanum nigrum; Solanum tuberosum; Spinacia oleracea; Stellaria media; Trifolium pratense; Trifolium repens; Tropaeolum majus; Tulipa; Vicia faba; Vicia villosa; Viola arvensis.

A further aspect of the present invention provides a method which includes causing or allowing transcription from a construct as disclosed within the genome of a plant cell to produce a cytoplasmically-replicating RNA.

A further aspect of the present invention provides a method of reducing or suppressing or lowering the level of a target gene in a plant cell, the method including causing or allowing transcription from a vector as disclosed above.

In preferred forms the present invention is concerned with providing amplicon-based methods are useful in functional genomics.

Thus in one aspect of the present invention, the target gene may be of unknown phenotype, in which case the TRV amplicon system may be employed to analyse the phenotype by generating a widespread null (or nearly null) phenotype. The target gene may be essential, which is to say that the null phenotype is lethal to the cell or tissue in question.

This aspect of the invention may comprise a method of characterizing a target gene comprising the steps of:

(a) silencing the target gene in a part or at a certain development stage of the plant using the TRV amplicon system described above,

(b) observing the phenotype of the part of the plant in which, or when, the target gene has been silenced.

Generally the observation will be contrasted with a plant wherein the target gene is being expressed in order to characterise (i.e. establish one or more phenotypic characteristics of) the gene.

The potential advantage of the TRV system over certain prior art constructs is discussed above. There are also several advantages of the current method over alternative methods in which the targeted gene is inactivated by insertional or other mutagenic procedures. The advantage over mutagenic procedures applies when there is more than one homologous gene carrying out the role of the target gene. Mutagenic procedures will not normally reveal a phenotype in that situation. A second situation where the current invention has advantage over both mutagenic and unregulated gene silencing procedures applies when the target gene has a lethal phenotype. The controllable attribute of the gene silencing will allow the phenotype of such genes to be investigated and exploited more efficiently than using the alternative methods available prior to the disclosure of the current invention.

Nor, for the identification of endogenous genes, would it be necessary to try and generate a transgenic plant in which gene silencing is already activated to observe the effect, although transgenic plants may be used if required.

In a further aspect there is disclosed a method of altering the phenotype of a plant comprising use of the silencing method discussed above. Traits for which it may be desirable to change the phenotype include the following: colour; disease or pest resistance; ripening potential; male sterility.

In a further aspect of the present invention there is disclosed a kit comprising a vector as described above.

In a further aspect of the present invention there is disclosed a host cell including a vector according to the present invention. These may be plant cells, or may be microbial (particularly bacterial and especially Agrobacterium) cells. Use of vector as described above in the transformation (stable or transient) of a plant is also embraced by the invention. The host cell may have incorporated into its genome a construct as described above.

In a further aspect there is disclosed a plant, or plant tissue, stably or transiently transformed by, a vector of the present invention. Thus in addition to a plant, the present invention provides any clone of such a plant, selfed or hybrid progeny and other descendants, and any part of any of these, such as propagules, (any part which may be used in reproduction or propagation, sexual or asexual, including cuttings, seed and so on). Plant extracts and derivatives are also provided. In each case the material will include, or be transformed by, the vector of the present invention.

The invention will now be further described with reference to the following non-limiting Figures and Examples. Other embodiments of the invention will occur to those skilled in the art in the light of these.

### SEQUENCES, FIGURES AND EXAMPLES

### SEQUENCE APPENDICES

The sequence of pBTAΔMP is given in full, including vector backbone. For the sequences of pBTAΔMPΔ16K, pBTAΔREPΔMP, and pBTAΔREPΔMPΔ16K, the vector backbone is not given.

1. pBINTRA6 (SEQ ID NO: 1)

2. pBTAΔMP (SEQ ID NO: 2)

3. pBTAΔMPΔ16K (SEQ ID NO: 3)

4. pBTAΔREPΔMP (SEQ ID NO: 4)

5. pBTAΔREPΔMPΔ16K (SEQ ID NO: 5)

6. A. thaliana partial cDNA sequence sulphur gene (SEQ ID NO: 6)

7. A. thaliana partial CDNA sequence RUBISCO small subunit gene (SEQ ID NO: 7)

8. A. thaliana partial cDNA sequence LEAFY gene (SEQ ID NO: 8)

9. mGFP5 cDNA sequence (SEQ ID NO: 9)

### FIGURES

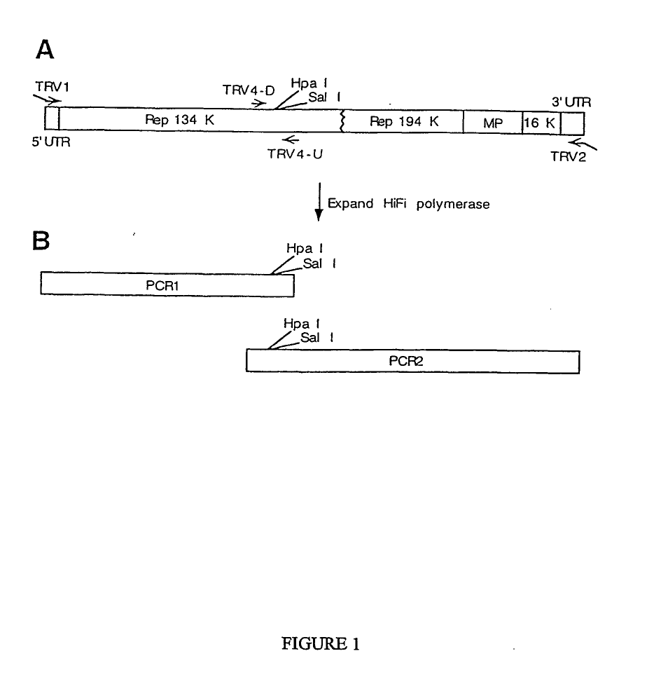

FIG. 1.

A; Schematic drawing of TRV RNA1; 5′UTR and 3′UTR are the 5′ and 3′ untranslated regions respectively; Rep 134 K is the 134KDa replicase protein; Rep 194 K is the 194 KDa read-through replicase protein; MP is the movement protein; 16K is the 16 KDa protein. B; The relative positions of the PCR1 and PCR2 cDNA fragments.

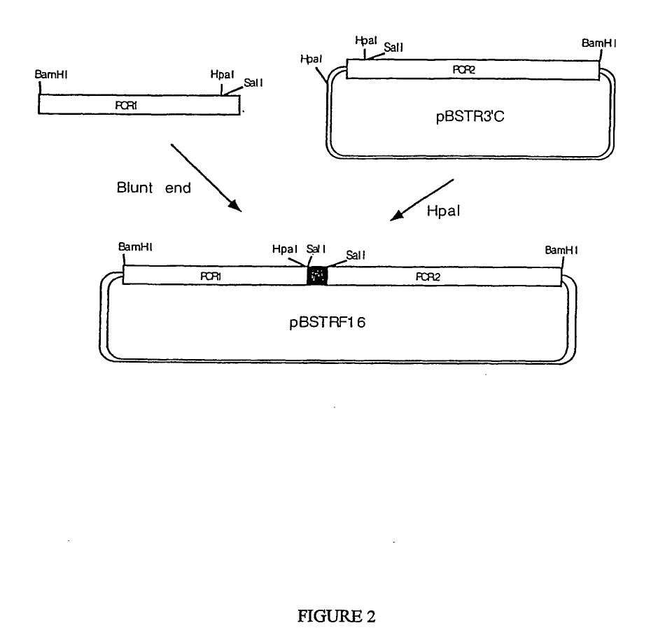

FIG. 2.

Schematic illustration of the cloning strategy for pBSTRF16.

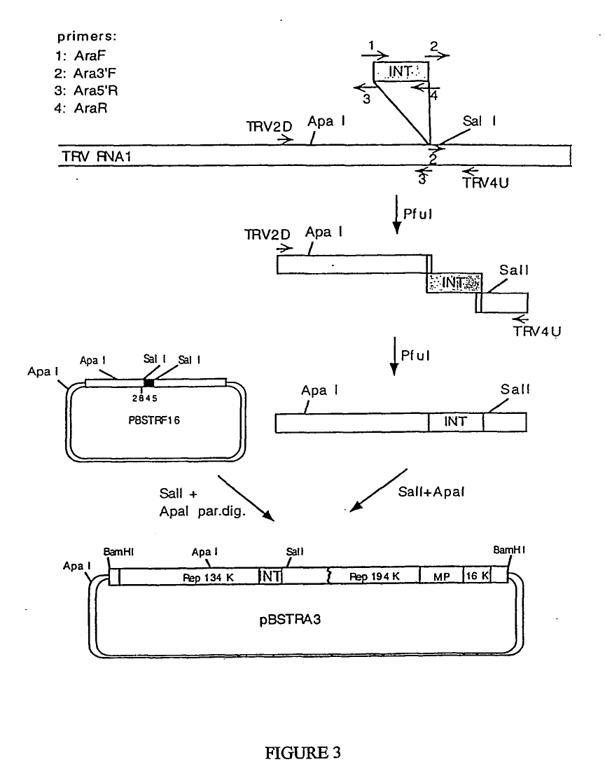

FIG. 3.

Schematic representation of the cloning strategy for the introduction of intron 3 from A. thaliana NIA1 gene to TRV RNA 1, to obtain pBSTRA3. INT is NIA1 gene intron 3.

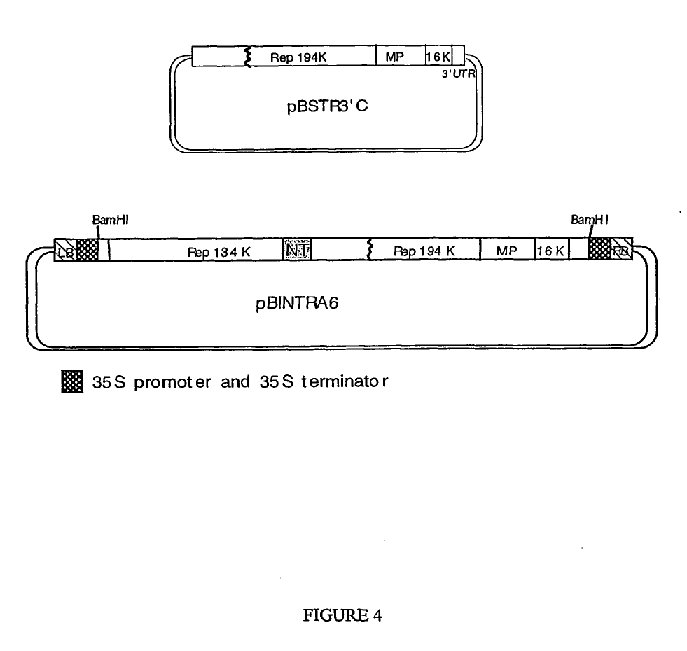

FIG. 4.

Schematic representation of pBSTR3′C and pBINTRA6. LB and RB respectively are the left border and right border of pBINTRA6 T-DNA.

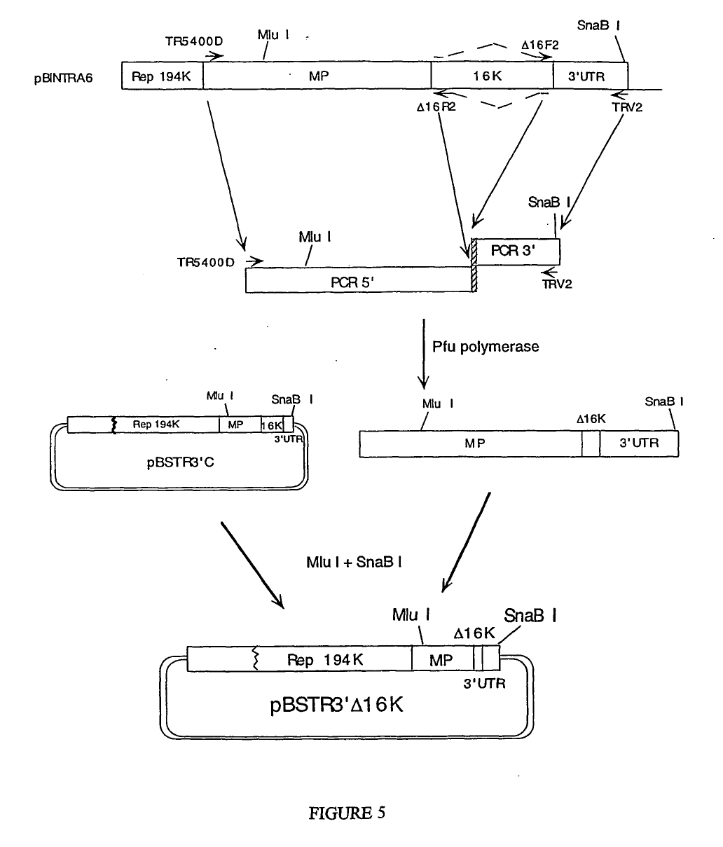

FIG. 5.

Schematic representation of the construction of pBSTR3′Δ16, plasmid carrying a deletion in the 16K gene. Rep 194K is the 3′ end of viral replicase. MP is the movement protein. 16K is the 16 kD protein gene. 3′ UTR is the 3′ untranslated region. Δ16K is the remaining part of the 16K gene.

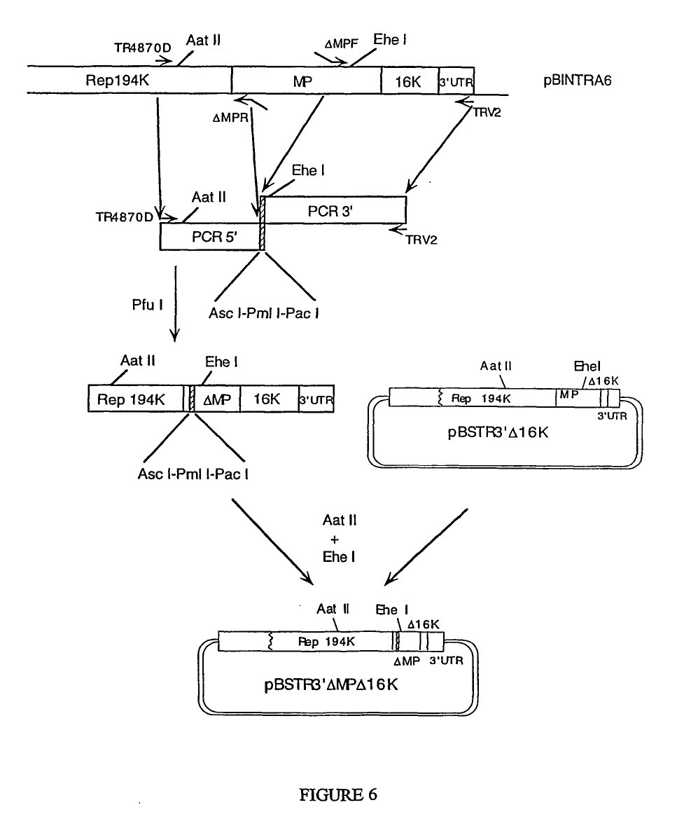

FIG. 6.

Schematic representation of the construction of pBSTR3′ΔMPΔ16K. AscI, PmlI and PacI are the restriction enzyme sites introduced into the multiple cloning site. ΔMP is the remaining part of the MP.

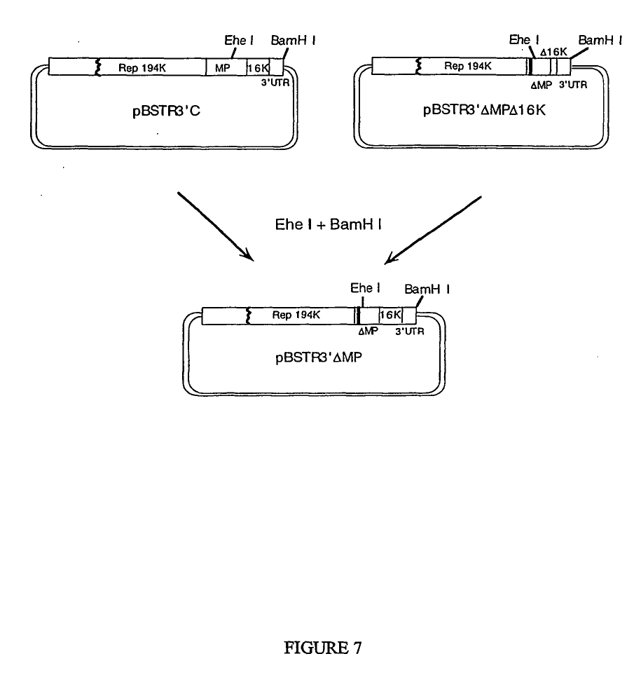

FIG. 7.

Schematic representation of the construction of pBSTR3′ΔMP.

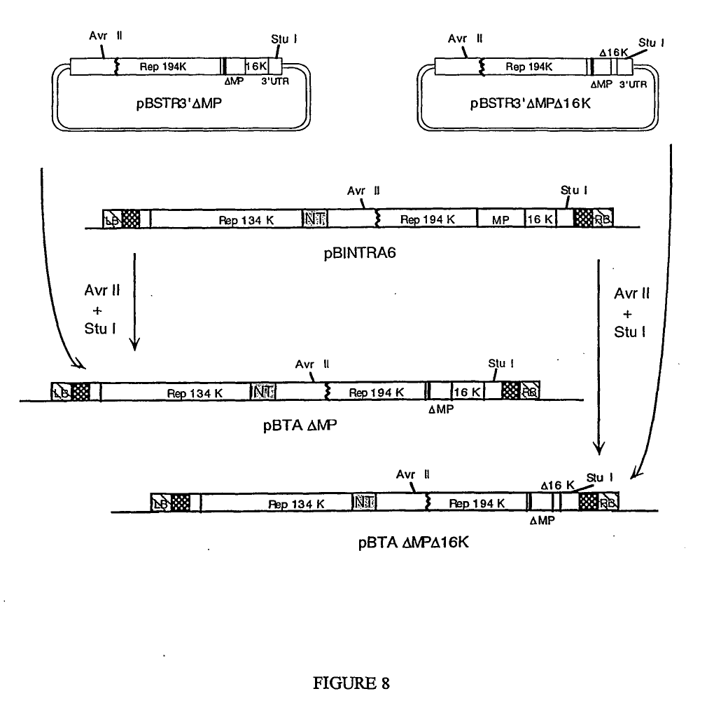

FIG. 8.

Schematic representation of the construction of the binary constructs pBTAΔMP and pBTAΔMPΔ16K.

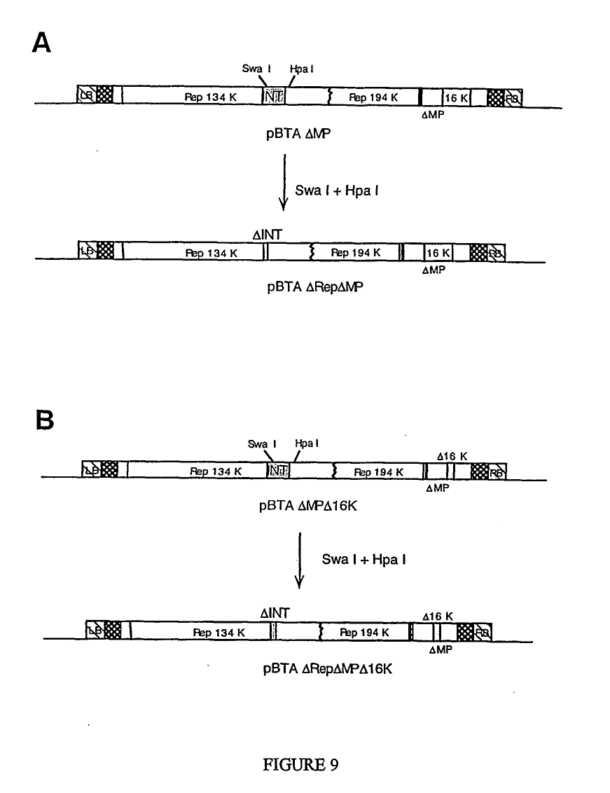

FIG. 9.

Construction of negative controls pBTAΔREPΔMP (A) and pBTAΔREPΔMPΔ16K (B). ΔINT is the remaining part of the intron. ΔRep is the remaining part of the viral replicase.

### EXAMPLES

### General Materials and Methods

Unless stated otherwise, all DNA modifications and digestions were performed using enzymes according to the manufacturers' instructs and following protocols described by Sambrook et al. (Sambrook, Fritsch et al. 1989). Unless stated otherwise, For cloning in E. coli, single or low copy-number vectors were preferred.

Construction pBINTRA6 and pBSTR3′C

pBINTRA6 is a full length infectious clone of TRV (strain PPK20) RNA1.

All the manipulations in TRV RNA1 had to be done first in the plasmid pBSTR3′C because it has more unique sites than pBINTRA6. The vectors were constructed as follows:

Total RNA was prepared from TRV (strain ppk20) infected N. benthamiana plants as previously described (Devic, Jaegle et al. 1989). Full length cDNA corresponding to TRV RNA1 was prepared from this RNA using Superscript Reverse Transcriptase (Gibco) and the primer TRV2 5′ggggggatccgggcgtaataacgcttacg3′ (SEQ ID NO: 10) which anneals to the 3′ end of TRV RNA1. All primers in this work were derived from the sequence of a closely related TRV strain SYM (Hamilton, Boccara et al. 1987) The full-length cDNA was used as a template for PCR amplification of two overlapping fragments, PCR1 and PCR2, which together cover all of TRV RNA1.

PCR1, a 3.2 kb fragment, was amplified using Expand HiFi polymerase (Roche). The primers were: TRV1 ′ggggggatccataaaacatttcaatcctttg3′ (SEQ ID NO: 11) (which anneals to positions 1-21 of TRV) and TRV4U 5′ttagcaccagctatctgagcgc3′ (SEQ ID NO: 12) (positions 3168-3189). PCR2, a 4.1 kb product, was also amplified using Expand HiFi polymerase (Roche) and the primers TRV4D 5′gttccaaccagacaaacgtatgg3′ (SEQ ID NO: 13) (positions 2698-2720) and TRV2 (see above).

PCR1 and PCR2 share a 491 nt overlap in the replicase open reading frame (ORF). The primers TRV1 and TRV2 contain BamHI sites to allow cloning of the full-length product (FIG. 1).

PCR2 was blunt-ended using T4 DNA polymerase, digested with BamHI, and cloned into the plasmid pBAC/SacB1 (Bendahmane, Kanyuka et al. 1999) which had previously digested with BamHI and EheI to form pBSTR3′ C. The PCR1 fragment was blunted-ended with T4 DNA polymerase and ligated into HpaI digested-pBSTR3′C, to form pBSTRF16. pBSTRF16 therefore contains 302 bp that are duplicated within the replicase ORF (FIG. 2).

The 302 bp of duplicated replicase sequence was replaced with a 438 bp intron. Intron 3 of Arabidopsis thaliana Col-0 nitrate reductase NIA1 gene (Wilkinson and Crawford 1993) was amplified using the primers AraF and AraR. AraF is 5′CGTATCTTTGCAA TAACAGgtaataatcctctctcttgatatt3′ (SEQ ID NO: 14), where the sequence in upper case corresponds to positions 2826-2845 of TRV RNA1 and the sequence in lower case corresponds to positions 1-24 of the intron. Similarly, AraR is 5′TTAAATTGTCCAAGATCAACct gtttaacacaagtcaacgtc3′ (SEQ ID NO: 15) where the sequence in upper case corresponds to positions 2846-2864 of TRV RNA 1 and the sequence in lower case corresponds to positions 416-438 of the intron. The PCR amplified intron 3 fragment was therefore flanked by the AGGT intron splice-sites, and 19 bp of TRV (exon) sequence (FIG. 3).

Two TRV-exons (exon 1 and exon 2) that flank the intron insertion site were then PCR amplified. For exon 1 the primers were TRV2D 5′tcgcacaaaaccaaggtgatag3′ (SEQ ID NO: 16) (positions 1772-1793) and Ara5′R 5′ggattatt acCTGTTATTGCAAAGATACGTCTG3′ (SEQ ID NO: 17) where the sequence in lower case corresponds to positions 1-10 of the intron and sequence in upper case corresponds to positions 2822-2845 of TRV RNA1. Exon 1 was amplified as a 1.07 kb fragment from pBSTR16. For exon 2 the primers were Ara3′F 5′tgttaaacagGTTGATC TTGGACAATTTAAGTGC3′ (SEQ ID NO: 18), where the sequence in upper case corresponds to positions 2846-2868 of TRV RNA1 and the sequence in lower case corresponds to positions 428-438 of the intron, and TRV4U (see above). Exon 2 was amplified as a 0.35 kb fragment from PCR 1 (see above). Exon 1, intron3 and exon 2 were all amplifed using Pfu polymerase (Promega). To introduce intron 3 to the TRV RNA 1 genome, chimeric PCR was performed with Pfu polymerase and the primers TRV2D and TRV4U using a mixture of exon 1, intron 3 and exon 2 as template to give a 1.8 kb fragment.

This 1.8 kb intron-containing-fragment was digested with ApaI and SalI and cloned in pBSTRF16 using ApaI-partial digestion and SalI, thus replacing the region that included duplicated sequence, and forming pBSTRA3 (FIG. 3).

To transfer the cloned RNA1 to a binary transformation vector, the 7.2 kb fragment corresponding to TRV RNA 1 was released from pBSTRA3 with BamHI and cloned into the BamHI site between the CaMV 35s promoter and the CaMV terminator on the plasmid pBIN61 to form pBINTRA6. pBIN61 is a modified version of the pBIN19 (Frisch, Harris-Haller et al. 1995) binary vector that carries a transcription cassette comprising the CaMV 35S promoter and terminator. To construct the pBIN61 binary vector, the transcription cassette containing the CaMV 35S promoter and terminator was released by digestion with KpnI and XhoI from the plasmid pJIT61 (kindly provided by P. Mullineaux, JIC, Norwich, UK). The transcription cassette was then ligated to the pBIN19 plasmid vector digested with KpnI and SalI to create pBIN61. pBIN61 is a low copy number vector in E. coli (10-15 copies per cell) in which the TRV insert can be stably cloned.

Agrobacterium strain GV3101 containing pBINTRA6 was infiltrated into N. benthamiana leaves causing a TRV RNA 1 infection. The full sequence of pBINTRA6 is given in the Appendix

A schematic representation of pBSTR3′C and pBINTRA6 is shown in FIG. 4.

Construction of pBSTR3′Δ16

First of all, we had to build a pBSTR3′C derivative bearing a deletion in the 16K sequence so that subsequent manipulations could be done on this derivative, in addition to pBSTR3′C, in order to produce corresponding amplicon vectors with and without the 16K open reading frame.

Most of the 16K open reading frame was removed from pBSTR3′C via chimeric PCR (Ho et al., 1989; Horton et al., 1989). Two fragments were amplified using as template pBINTRA6 and Pfu I polymerase (Promega). The 5′ PCR fragment was amplified using primers TR5400D: 5′ ttctcaaatctaggggccattg 3′ (SEQ ID NO: 19) corresponding to positions 5381 to 5403 of TRV RNA1 and Δ16R2: 5′ CCGAAAGGAACacttcattcacacaacccttga 3′ (SEQ ID NO: 20), were letters in upper case correspond to positions 6501 to 6511 of TRV RNA1, and letters in lower case correspond to positions 6124 to 6145. This fragment was 0.77 Kb. The 3′ PCR fragment was amplified using primers Δ16F2: 5′ gaatgaagtGTTCCTTTCGGGATTGATCGTT 3′ (SEQ ID NO: 21) where the letters in upper case correspond to positions 6501 to 6522 and the letters in lower case, to positions 6137 to 6145 and TRV2: 5′ggggggatccgggcgtaataacgcttacg3′ (SEQ ID NO: 10) which anneals to the 3′ end of TRV RNA1 (positions 6770-6789). This fragment was 0.3 Kb. Both fragments, therefore, share an overlapping sequence of 20 nucleotides. To produce the deletion, chimeric PCR was performed with Pfu I polymerase and primers TR5400D and TRV2 using a mixture of 5′ and 3′ PCR fragments to give a fragment of 1.07 Kb in which 355 bp from 16Kb open reading frame have been deleted.

To insert this deletion into pBSTR3′C, the PCR fragment was digested with MluI and SnaBI and inserted in the MluI and SnaBI sites of pBSTR3′C to give the plasmid pBSTR3′Δ16 (FIG. 5).

Construction of pBTAΔMP and pBTAΔMPΔ16K

Part of the movement protein sequence was removed from pBINTRA6 and a multiple cloning site was engineered and put in its place via chimeric PCR (FIG. 6).

To carry out the deletion two fragments were amplified using PfuI polymerase and pBINTRA6 as template. The 5′ PCR fragment was amplified using primers TR4870D: 5′actcactgattgcgtttcctag 3′ (SEQ ID NO: 22) (positions 4848-4869) and ΔMPR: 5′ ttaattaacacgtggcgcgccAGTCTTCTTCTTCAAGGTGACC 3′ (SEQ ID NO: 23), where the sequence in lower case corresponds to the sequence of AscI-PmlI-PacI sites of the engineered polylinker and the sequence in upper case, to positions 5345 to 5366 of TRV RNA1. This fragment was 0.54Kb. The 3′ PCR fragment was amplified using primers ΔMPF: 5′ ggcgcgccacgtgttaattaaCTGATTCGACTAGGCGCCTC 3′ (SEQ ID NO: 24), where the sequence in lower case corresponds to the sequence of AscI-PmlI-PacI sites of the engineered polylinker and the sequence in upper case, to positions 5857 to 5876. and TRV2 (see above). This fragment was 0.96 Kb. Both fragments share a 21 nucleotides fragment corresponding to the engineered polylinker. The actual deletion and introduction of the polylinker was made via chimeric PCR using PfuI polymerase and primers TR4870 and TRV2 and a mixture of both PCR fragments. The product was 1.5 Kb. Then, it was digested with AatII and EheI and introduced into the AatII and EheI sites of pBSTR3′Δ16 to produce pBSTR3′ΔMPΔ16 (FIG. 6).

To produce the corresponding construct carrying only the deletion in the MP gene and not in the 16K gene, pBSTR3′ΔMPΔ16 was digested with EheI and BamHI to remove a 568 bp fragment including the 16K deletion and replaced by a 923 bp BamHI-EheI fragment from pBSTR3′C carrying the full length 16K gene (FIG. 7).

To introduce these deletions into the binary construct pBINTRA6, pBSTR3′Δ16 and pBSTR3′ΔMPΔ16 were digested with AvrII and StuI and the fragments containing the deletions were cloned into the AvrII and StuI sites of pBINTRA6 to produce pBTAΔMP and pBTAΔMPΔ16 (FIG. 8)

Construction of the Negative Control Amplicon Vectors

The corresponding negative control, non replicative vectors bearing a deletion on the viral replicase gene were constructed by digesting both pBTAΔMP and pBTAΔMPΔ16 with SwaI and HpaI, which have unique sites on these vectors and produce blunt ends. Then the resulting fragment was religated, to produce either pBTAΔRepΔMP or pBTAΔRepΔMPΔ16. Since the SwaI site was inside the intron, these constructs have lost 368 bp of the intron and 40 bp of the replicase. They have also lost one of the intron splicing sites and, therefore, will be unable to splice the intron to produce a native replicase (FIGS. 9A and 9B).

Construction of Amplicon-Derived Constructs

Sulphur Constructs

Sulphur codifies for a magnesium chelatase, an enzyme required for chlorophyll formation. A 944 bp cDNA fragment was amplified from pTV09 plasmid (F. Ratcliff, unpublished), which contained a 1.2 Kb of A. thaliana cDNA sulphur gene (Kjemtrup et al., 1998). It was amplified using Expand HiFi polymerase and the primers SUL1: 5′ ccttggcgcgccttcactctcttctccttcc (SEQ ID NO: 25) and SUL2: 5′ ccccttaattaatctggtcttgaagcttgtcc (SEQ ID NO: 26). SUL1 carries a restriction site for AscI and SUL2, one for PacI to facilitate the insertion of the fragment into the AscI and PacI sites of the multiple cloning site of the amplicon vectors The resulting constructs were pBTAΔMP:S, pBTAΔMPΔ16K:S, pBTAΔREPΔMP:S and pBTAΔREPΔMPΔ16:S. The sequence is given in the Appendix.

RUBISCO Constructs

RUBISCO is a gene involved in carbon fixation during photosynthesis. A 469 bp cDNA fragment of the rubisco small sub-unit was PCR amplified from A. thaliana cDNA using Expand HiFi polymerase and the primers aRUB1:5′ ccttggcgcgcctctatgctctcttccgcta (SEQ ID NO: 27) and aRUB2:5′ ccccttaattaatccgatgatcctaatgaaggc (SEQ ID NO: 28). As above, the primers carry restriction sites for AscI and PacI to facilitate the cloning into the corresponding AscI and PacI sites of the multiple cloning site of the amplicon vectors. The resulting constructs were pBTAΔMP:aR, pBTAΔMPΔ16K:aR, pBTAΔREPΔMP:aR and pBTAΔREPΔMPΔ16:aR. The sequence is given in the Appendix.

LEAFY Constructs

A 940 bp cDNA fragment of LEAFY, a gene involved in floral development in A. thaliana, was PCR amplified from plasmid pDW122 (Weigel et al., 1992) using Expand HiFi and the primers LEAFY1: 5′ ccttggcgcgccatacggtatacgtttctacac (SEQ ID NO: 29) and LEAFY2: 5′ ccccttaattaaagacggcgtctatatccc (SEQ ID NO: 30). As above, the primers carry restriction sites for AscI and PacI to facilitate the cloning into the corresponding AscI and PacI sites of the multiple cloning site of the amplicon vectors. The resulting constructs were pBTAΔMP:Lfy, pBTAΔMPΔ16K:Lfy, pBTAΔREPΔMP:Lfy and pBTAΔREPΔMPΔ16:Lfy. The sequence is given in the Appendix.

GFP Constructs p A 790 bp fragment containing the whole coding sequence of mGFP5 was amplified from plasmid CL106 (Haseloff et al., 1997) using Expand HiFi polymerase and the primers 5′GFP: 5′ ggttggcgcgccaatgaagactaatctttttctc (SEQ ID NO: 31) and 3′GFP: 5′ ggggttaattaattagagttcgtcatgtttgta (SEQ ID NO: 32). As above, the primers carry restriction sites for AscI and PacI to facilitate the cloning into the corresponding AscI and PacI sites of the multiple cloning site of the amplicon vectors. This way, the GFP gene is in frame with the first 13 amino acids of the movement protein and will be expressed as a fusion protein. The resulting constructs were pBTAΔMP:GFP, pBTAΔMPΔ16K:GFP, pBTAΔREPΔMP:GFP and pBTAΔREPΔMPΔ16:GFP. The sequence is given in the Appendix.

To carry out the transient assays in N benthamiana and A. thaliana transformation, all the amplicon derived constructs were introduced into the Agrobacterium strain GV3101.

Plant Manipulations

Plant Growth Conditions

All work involving virus infected material was carried out in containment glasshouses under MAFF license PHF 1420c/1773(December 1996). N. benthamiana and A. thaliana plants were germinated on a 1:1 mixture of JIC compost and peat, then grown individually in pots at 25° C. during the day and 20° C. during the night. Supplementary winter lighting from halogen quartz iodide lamps provided a 16 hour day length.

Agrobacterium-Infiltration

Virus infections on N. benthamiana were achieved by Agrobacterium-mediated transient gene expression of infectious constructs from the T-DNA of a binary plasmid (e.g. any of the amplicon constructs). Agrobacterium was grown to saturation in L broth. The culture was then centrifuged and re-suspended in 10 mM MgCl 2, 10 mM MES and 150 mM acetosyringone, and kept at room temperature for 2 hours. The culture was then infiltrated to the underside of a leaf using a 2 ml syringe without a needle.

### Example 1

Transient Assay to Demonstrate Replication

The ability of the amplicon constructs to replicate in plants is tested on N. benthamiana as follows. Agrobacterium cultures of amplicon constructs carrying the whole GFP gene (pBTAΔMP:GFP, pBTAΔMPΔ16:GFP, pBTAΔREPΔMP:GFP, pBTAΔREPΔMPΔ16:GFP) are infiltrated into all the leaves of N benthamiana plants four weeks old. Ten days after infiltration, the infiltrated patch shows green fluorescence under UV light. Controls unable to replicate do not show green fluorescence in the infiltrated patch. Samples may be taken to confirm the presence of GFP RNA in those plants using northern blotting.

### Example 2

Transient Assay to Demonstrate Silencing

Ability to produce silencing may be tested on N benthamiana plants as follows. Agrobacterium cultures of amplicon constructs carrying a piece of sulphur gene are infiltrated into all the leaves of N. benthamiana plants four weeks old. Ten days after infiltration the infiltrated patch shows a faint yellow colour typical representing sulphur-silencing in the leaves. Controls unable to replicate, or having weaker promoters, show reduced silencing or no silencing in the infiltrated patch. Samples may be collected to confirm the absence of sulphur RNA from silenced plants using northern blotting.

Corresponding assays employing GFP-based constructs, in GFP-transgenic N benthamiana plants, may also be performed.

### Example 3

Transformation of A thaliana with Amplicons

GV3101 Agrobacterium cultures containing individual amplicon constructs were grown in 500 ml L broth in the presence of 50 μg/ml Kanamycin at 28° C. After centrifugation at room temperature the cells were resuspended in 400 ml of infiltration medium (2.2 g Murashige and Skoog medium, 50 g sucrose, 50 μl Silwet copolymer L-77, 10 μl of 1 mg/ml BAP and 0.5 g MES per litre). Flowering A. thaliana plants were immersed for 1 minute into the suspension by inverting the pot and then left standing, covered with a plastic bag, to maintain the humidity. Next day the plastic bag was removed. This was done with 5 pots per construct. Two ecotypes, c-24 and Col-0 were transformed with the amplicon constructs this way.

### REFERENCES

Inasmuch as they may be required by the person skilled in the art to practice the present invention, all citations are specifically included herein by cross-reference.

Angell, S. M., and Baulcombe, D. C. (1997). Consistent gene silencing in transgenic plants expressing a replicating potato virus X RNA. EMBO J. 16, 3675-3684.

Angell, S. M., and Baulcombe, D. C. (1999). Potato virus X amplicon-mediated silencing of nuclear genes. Plant J. 20, 357-362.

Atkinson, R. G., Bieleski, L. R. F., Gleave, A. P., Jannsen, B. J., and Morris, B. A. M. (1998). Post-transcriptional silencing of chalcone synthase in petunia using a geminivirus-based episomal vector. Plant J. 15, 593-604.

Dalmay, T., Hamilton, A. J., Mueller, E., and Baulcombe, D. C. (2000). Potato Virus X amplicons in arabidopsis mediate genetic and epigenetic gene silencing. Plant Cell 12, 369-380.

Devic, M., M. Jaegle, et al. (1989). “Symptom production on tobacco and tomato is determined by two distinct domains of the satellite RNA of cucumber mosaic virus (strain Y).” J.Gen.Virol. 70: 2765-2774.

Frisch, D. A., L. W. Harris-Haller, et al. (1995). “Complete Sequence of the binary vector Bin 19.” Plant Molecular Biology 27: 405-409.

Hamilton, W. D. O., M. Boccara, et al. (1987). “The complete nucleotide sequence of tobacco rattle virus RNA-1.” J.Gen.Virol. 68: 2563-2575.

Haseloff, J., Siemering, K. R., Prasher, D. C., and Hodge, S. (1997). Removal of a cryptic intron and subcellular localization of green fluorescent protein are required to mark transgenic Arabidopsis plants brightly. Proc. Natl. Acad. Sci. USA 94, 2122-2127.

Ho, S., Hunt, H. D., Horton, R. M., Pullen, K. J., and Peare, L. R. (1989). Site-directed mutagenesis by overlap extension using the PCR. Gene 77, 51-59.

Horton, R. M., Hunt, H. D., Ho, S. N., Pullen, J. K., and Pease, L. R. (1989). Engineering hybrid genes without the use of restriction enzymes: gene splicing by overlap extension. Gene 77, 61-68.

Kaido, M., Mori, M., Mise, K., Okuno, T., and Furusawa, I. (1995). Inhibition of brome mosaic virus (BMV) amplification in protoplasts from transgenic tobacco plants expressing replicable BMV RNAs. J.Gen.Virol. 76, 2827-2833.

Kjemtrup, S., Sampson, K. S., Peele, C. G., Nguyen, L. V., Conkling, M. A., Thompson, W. F., and Robertson, D. (1998). Gene silencing from plant DNA carried by a geminivirus. Plant J. 14, 91-100.

Kumagai, M. H., Donson, J., Della-Cioppa, G., Harvey, D., Hanley, K., and Grill, L. K. (1995). Cytoplasmic inhibition of carotenoid biosynthesis with virus-derived RNA. Proc.Natl.Acad.Sci.USA 92, 1679-1683.

Mori, M., Zhang, G.-H., Kaido, M., Okuno, T., and Furusawa, I. (1993). Efficient production of human gamma interferon in tobacco protoplasts by genetically engineered brome mosaic virus RNAs. J.Gen.Virol. 74, 1255-1260.

Napoli, C., Lemieux, C., and Jorgensen, R. A. (1990). Introduction of a chimeric chalcone synthase gene into Petunia results in reversible co-suppression of homologous genes in trans. Plant Cell 2, 279-289.

Ruiz, M. T., Voinnet, O., and Baulcombe, D. C. (1998). Initiation and maintenance of virus-induced gene silencing. Plant Cell 10, 937-946.

van der Krol, A. R., Mur, L. A., Beld, M., Mol, J. N. M., and Stuitji, A. R. (1990). Flavonoid genes in petunia: Addition of a limited number of gene copies may lead to a suppression of gene expression. Plant Cell 2, 291-299.

Weigel, D., Alvarez, J., Smyth, D. R., Yanofsky, M. F., and Meyerowitz, E. M. (1992). LEAFY Controls Floral Meristem Identity in Arabidopsis. Cell 69, 843-859.

Yamaya, J., Yoshioka, M., Meshi, T., Okada, Y., and Ohno, T. (1988). Cross protection in transgenic tobacco plants expressing a mild strain of tobacco mosaic virus. Mol.Gen.Genet. 215, 173-175.

Sequence Appendix

Key to the sequence annotation:

| | |
|---|---|
| Lower-case | plasmid backbone sequence. |
| Lower-case italics | sequence inserted into the amplicon |
| | constructs. |
| Lower-case underlined | CaMV 35S promoter sequence. |
| UPPER-CASE | tobacco rattle virus RNA1 cDNA sequence. |
| UPPER-CASE AND BOLD | Arabidopsis NIA1-intron 3 sequence. |
| UPPER-CASE ITALICS | |
| UNDERLINED | multiple cloning site. |
| Lower-case and bold | CaMV 35S terminator sequence. |
| . . . | deletion oE 16K. |
| (. . . ) | deletion of the replicase and |
| | intron. |

The sequence of pBTAΔMP is given in full, including the vector backbone. For the sequence of the other three amplicon constructs (pBTAΔMPΔ16K, pBTAΔRepΔMP and pBTAΔRepΔMPΔ16K) vector backbone is not given, since is the same for all of them.

SEQ ID NO 1 - Complete Sequence of pBINTRA6 Plasmid.
tactccaaaaatgtcaaagatacagtctcagaagaccaaagggctattgagacttttcaacaaaggg
taatttcgggaaacctcctcggattccattgcccagctatctgtcacttcatcgaaaggacagtaga
aaaggaaggtggctcctacaaatgccatcattgcgataaaggaaaggctatcattcaagatgcctct
gccgacagtggtcccaaagatggacccccacccacgaggagcatcgtggaaaaagaagacgtcccaa
ccacgtcttcaaagcaagtggattgatgtgacatctccactgacgtaagggatgacgcacaatccca
ctatccttcgcaagacccttcttctatataaggaagttcatttcatttggagaggacagcccaagct
ttctagagGATCCATAAAACATTTCAATCCTTTGAACGCGGTAGAACGTGCTAATTGGATTTTGGTG
AGAACGCGGTAGAACGTACTTATCACCTACAGTTTTATTTTGTTTTTCTTTTTGGTTTAATCTATCC
AGCTTAGTACCGAGTGGGGGAAAGTGACTGGTGTGCCTAAAACCTTTTCTTTGATACTTTGTAAAAA
TACATACAGATACAATGGCGAACGGTAACTTCAAGTTGTCTCAATTGCTCAATGTGGACGAGATGTC
TGCTGAGCAGAGGAGTCATTTCTTTGACTTGATGCTGACTAAACCTGATTGTGAGATCGGGCAAATG
ATGCAAAGAGTTGTTGTTGATAAAGTCGATGACATGATTAGAGAAAGAAAGACTAAAGATCCAGTGA
TTGTTCATGAAGTTCTTTCTCAGAAGGAACAGAACAAGTTGATGGAAATTTATCCTGAATTCAATAT
CGTGTTTAAAGACGACAAAAACATGGTTCATGGGTTTGCGGCTGCTGAGCGAAAACTACAAGCTTTA
TTGCTTTTAGATAGAGTTCCTGCTCTGCAAGAGGTGGATGACATCGGTGGTCAATGGTCGTTTTGGG
TAACTAGAGGTGAGAAAAGGATTCATTCCTGTTGTCCAAATCTAGATATTCGGGATGATCAGAGAGA
AATTTCTCGACAGATATTTCTTACTGCTATTGGTGATCAAGCTAGAAGTGGTAAGAGACAGATGTCG
GAGAATGAGCTGTGGATGTATGACCAATTTCGTGAAAATATTGCTGCGCCTAACGCGGTTAGGTGCA
ATAATACATATCAGGGTTGTACATGTAGGGGTTTTTCTGATGGTAAGAAGAAAGGCGCGCAGTATGC
GATAGCTCTTCACAGCCTGTATGACTTCAAGTTGAAAGACTTGATGGCTACTATGGTTGAGAAGAAA
ACTAAAGTGGTTCATGCTGCTATGCTTTTTGCTCCTGAAAGTATGTTAGTGGACGAAGGTCCATTAC
CTTCTGTTGACGGTTACTACATGAAGAAGAACGGGAAGATCTATTTCGGTTTTGAGAAAGATCCTTC
CTTTTCTTACATTCATGACTGGGAAGAGTACAAGAAGTATCTACTGGGGAAGCCAGTGAGTTACCAA
GGGAATGTGTTCTACTTCGAACCGTGGCAGGTGAGAGGAGACACAATGCTTTTTTCGATCTACAGGA
TAGCTGGAGTTCCGAGGAGGTCTCTATCATCGCAAGAGTACTACCGAAGAATATATATCAGTAGATG
GGAAAGCATGGTTGTTGTCCCAATTTTCGATCTGGTCGAATCAACGCGAGAGTTGGTCAAGAAAGAC
CTGTTTGTAGAGAAACAATTCATGGACAAGTGTTTGGATTACATAGCTAGGTTATCTGACCAGCAGC
TGACCATAAGCAATGTTAAATCATACTTGAGTTCAAATAATTGGGTCTTATTCATAAACGGGGCGGC
CGTGAAGAACAAGCAAAGTGTAGATTCTCGAGATTTACAGTTGTTGGCTCAAACTTTGCTAGTGAAG
GAACAAGTGGCGAGACCTGTCATGAGGGAGTTGCGTGAAGCAATTCTGACTGAGACGAAACCTATCA
CGTCATTGACTGATGTGCTGGGTTTAATATCAAGAAAACTGTGGAAGCAGTTTGCTAACAAGATCGC
AGTCGGCGGATTCGTTGGCATGGTTGGTACTCTAATTGGATTCTATCCAAAGAAGGTACTAACCTGG
GCGAAGGACACACCAAATGGTCCAGAACTATGTTACGAGAACTCGCACAAAACCAAGGTGATAGTAT
TTCTGAGTGTTGTGTATGCCATTGGAGGAATCACGCTTATGCGTCGAGACATCCGAGATGGACTGGT
GAAAAAACTATGTGATATGTTTGATATCAAACGGGGGGCCCATGTCTTAGACGTTGAGAATCCGTGC
CGCTATTATGAAATCAACGATTTCTTTAGCAGTCTGTATTCGGCATCTGAGTCCGGTGAGACCGTTT
TACCAGATTTATCCGAGGTAAAAGCCAAGTCTGATAAGCTATTGCAGCAGAAGAAAGAAATCGCTGA
CGAGTTTCTAAGTGCAAAATTCTCTAACTATTCTGGCAGTTCGGTGAGAACTTCTCCACCATCGGTG
GTCGGTTCATCTCGAAGCGGACTGGGTCTGTTGTTGGAAGACAGTAACGTGCTGACCCAAGCTAGAG
TTGGAGTTTCAAGAAAGGTAGACGATGAGGAGATCATGGAGCAGTTTCTGAGTGGTCTTATTGACAC
TGAAGCAGAAATTGACGAGGTTGTTTCAGCCTTTTCAGCTGAATGTGAAAGAGGGGAAACAAGCGGT
ACAAAGGTGTTGTGTAAACCTTTAACGCCACCAGGATTTGAGAACGTGTTGCCAGCTGTCAAACCTT
TGGTCAGCAAAGGAAAAACGGTCAAACGTGTCGATTACTTCCAAGTGATGGGAGGTGAGAGATTACC
AAAAAGGCCGGTTGTCAGTGGAGACGATTCTGTGGACGCTAGAAGAGAGTTTCTGTACTACTTAGAT
GCGGAGAGAGTCGCTCAAAATGATGAAATTATGTCTCTGTATCGTGACTATTCGAGAGGAGTTATTC
GAACTGGAGGTCAGAATTACCCGCACGGACTGGGAGTGTGGGATGTGGAGATGAAGAACTGGTGCAT
ACGTCCAGTGGTCACTGAACATGCTTATGTGTTCCAACCAGACAAACGTATGGATGATTGGTCGGGA
TACTTAGAAGTGGCTGTTTGGGAACGAGGTATGTTGGTCAACGACTTCGCGGTCGAAAGGATGAGTG
ATTATGTCATAGTTTGCGATCAGACGTATCTTTGCAATAACAGGTAATAATCCTCTCTCTTGATATT
TTTAAATTATAGAATTAATTAGTTTACTTTATTCTTTACTATATGATTTAAATAGTTTAATCTTGTT
TTTGAGTAAACTATTCGATTTTGATATTTGTATTCGTCCTACAAAGTTGGAAATACTGATGATATTT
TCTTTTGAACGTGATACCTACCAATACTAATCTTACGGAATCTTTTAATAGAGCACTAATCAACATG
GAACTAAAGACCAATTCTTAAGTGTCTCTGTTGTACAGTTCATTTTAGTAGTGCGTTTAAGTATTAT
TATCTCCCTTCATGCGGGGCAATTATGTAGATTAAAATCGAAATTATATAAAATTTACATAAGTCTA
AGTCTAGGGTCTCCAGCTAATTGTTATTTTTTTAACGATGTTGACTAAAGCAATAACGACGTTGACT
TGTGTTAAACAGGTTGATCTTGGACAATTTAAGTGCCCTGGATCTAGGACCAGTTAACTGTTCTTTT
GAATTAGTTGACGGTGTACCTGGTTGTGGTAAGTCGACAATGATTGTCAACTCAGCTAATCCTTGTG
TCGATGTGGTTCTCTCTACTGGGAGAGCAGCAACCGACGACTTGATCGAGAGATTCGCGAGCAAAGG
TTTTCCATGCAAATTGAAAAGGAGAGTGAAGACGGTTGATTCTTTTTTGATGCATTGTGTCGATGGT
TCTTTAACCGGAGACGTGTTGCATTTCGACGAAGCTCTCATGGCCCATGCTGGTATGGTGTACTTTT
GCGCTCAGATAGCTGGTGCTAAACGATGTATCTGTCAAGGAGATCAGAATCAAATTTCTTTCAAGCC
TAGGGTATCTCAAGTTGATTTGAGGTTTTCTAGTCTGGTCGGAAAGTTTGACATTGTTACAGAAAAA
AGAGAAACTTACAGAAGTCCAGCAGATGTGGCTGCCGTATTGAACAAGTACTATACTGGAGATGTCA
GAACACATAACGCGACTGCTAATTCGATGACGGTGAGGAAGATTGTGTCTAAAGAACAGGTTTCTTT
GAAGCCTGGTGCTCAGTACATAACTTTCCTTCAGTCTGAGAAGAAGGAGTTGGTAAATTTGTTGGCA
TTGAGGAAAGTGGCAGCTAAAGTGAGTACAGTACACGAGTCGCAAGGAGAGACATTCAAAGATGTAG
TCCTAGTCAGGACGAAACCTACGGATGACTCAATCGCTAGAGGTCGGGAGTACTTAATCGTGGCATT
GTCGCGTCACACACAATCACTTGTGTATGAAACTGTGAAAGAGGACGATGTAAGCAAAGAGATCAGG
GAAAGTGCCGCGCTTACGAAGGCGGCTTTGGCAAGATTTTTTGTTACTGAGACCGTCTTATGACGGT
TTCGGTCTAGGTTTGATGTCTTTAGACATCATGAAGGGCCTTGCGCCGTTCCAGATTCAGGTACGAT
TACGGACTTGGAGATGTGGTACGACGCTTTGTTTCCGGGAAATTCGTTAAGAGACTCAAGCCTAGAC
GGGTATTTGGTGGCAACGACTGATTGCAATTTGCGATTAGACAATGTTACGATCAAAAGTGGAAACT
GGAAAGACAAGTTTGCTGAAAAAGAAACGTTTCTGAAACCGGTTATTCGTACTGCTATGCCTGACAA
AAGGAAGACTACTCAGTTGGAGAGTTTGTTAGCATTGCAGAAAAGGAACCAAGCGGCACCCGATCTA
CAAGAAAATGTGCACGCGACAGTTCTAATCGAAGAGACGATGAAGAAGCTGAAATCTGTTGTCTACG
ATGTGGGAAAAATTCGGGCTGATCCTATTGTCAATAGAGCTCAAATGGAGAGATGGTGGAGAAATCA
AAGCACAGCGGTACAGGCTAAGGTAGTAGCAGATGTGAGAGAGTTACATGAAATAGACTATTCGTCT
TACATGTATATGATCAAATCTGACGTGAAACCTAAGACTGATTTAACACCGCAATTTGAATACTCAG
CTCTACAGACTGTTGTGTATCACGAGAAGTTGATCAACTCGTTGTTCGGTCCAATTTTCAAAGAAAT
TAATGAACGCAAGTTGGATGCTATGCAACCACATTTTGTGTTCAACACGAGAATGACATCGAGTGAT
TTAAACGATCGAGTGAAGTTCTTAAATACGGAAGCGGCTTACGACTTTGTTGAGATAGACATGTCTA
AATTCGACAAGTCGGCAAATCGCTTCCATTTACAACTGCAGCTGGAGATTTACAGGTTATTTGGGCT
GGATGAGTGGGCGGCCTTCCTTTGGGAGGTGTCGCACACTCAAACTACTGTGAGAGATATTCAAAAT
GGTATGATGGCGCATATTTGGTACCAACAAAAGAGTGGAGATGCTGATACTTATAATGCAAATTCAG
ATAGAACACTGTGTGCACTCTTGTCTGAATTACCATTGGAGAAAGCAGTCATGGTTACATATGGAGG
AGATGACTCACTGATTGCGTTTCCTAGAGGAACGCAGTTTGTTGATCCGTGTCCAAAGTTGGCTACT
AAGTGGAATTTCGAGTGCAAGATTTTTAAGTACGATGTCCCAATGTTTTGTGGGAAGTTCTTGCTTA
AGACGTCATCGTGTTACGAGTTCGTGCCAGATCCGGTAAAAGTTCTGACGAAGTTGGGGAAAAAGAG
TATAAAGGATGTGCAACATTTAGCCGAGATCTACATCTCGCTGAATGATTCCAATAGAGCTCTTGGG
AACTACATGGTGGTATCCAAACTGTCCGAGTCTGTTTCAGACCGGTATTTGTACAAAGGTGATTCTG
TTCATGCGCTTTGTGCGCTATGGAAGCATATTAAGAGTTTTACAGCTCTGTGTACATTATTCCGAGA
CGAAAACGATAAGGAATTGAACCCGGCTAAGGTTGATTGGAAGAAGGCACAGAGAGCTGTGTCAAAC
TTTTACGACTGGTAATATGGAAGACAAGTCATTGGTCACCTTGAAGAAGAAGACTTTCGAAGTCTCA
AAATTCTCAAATCTAGGGGCCATTGAATTGTTTGTGGACGGTAGGAGGAAGAGACCGAAGTATTTTC
ACAGAAGAAGAGAAACTGTCCTAAATCATGTTGGTGGGAAGAAGAGTGAACACAAGTTAGACGTTTT
TGACCAAAGGGATTACAAAATGATTAAATCTTACGCGTTTCTAAAGGTAGTAGGTGTACAACTAGTT
GTAACATCACATCTACCTGCAGATACGCCTGGGTTCATTCAAATCGATCTGTTGGATTCGAGACTTA
CTGAGAAAAGAAAGAGAGGAAAGACTATTCAGAGATTCAAAGCTCGAGCTTGCGATAACTGTTCAGT
TGCGCAGTACAAGGTTGAATACAGTATTTCCACACAGGAGAACGTACTTGATGTCTGGAAGGTGGGT
TGTATTTCTGAGGGCGTTCCGGTCTGTGACGGTACATACCCTTTCAGTATCGAAGTGTCGCTAATAT
GGGTTGCTACTGATTCGACTAGGCGCCTCAATGTGGAAGAACTGAACAGTTCGGATTACATTGAAGG
CGATTTTACCGATCAAGAGGTTTTCGGTGAGTTCATGTCTTTGAAACAAGTGGAGATGAAGACGATT
GAGGCGAAGTACGATGGTCCTTACAGACCAGCTACTACTAGACCTAAGTCATTATTGTCAAGTGAAG
ATGTTAAGAGAGCGTCTAATAAGAAAAACTCGTCTTAATGCATAAAGAAATTTATTGTCAATATGAC
GTGTGTACTCAAGGGTTGTGTGAATGAAGTCACTGTTCTTGGTCACGAGACGTGTAGTATCGGTCAT
GCTAACAAATTGCGAAAGCAAGTTGCTGACATGGTTGGTGTCACACGTAGGTGTGCGGAAAATAATT
GTGGATGGTTTGTCTGTGTTGTTATCAATGATTTTACTTTTGATGTGTATAATTGTTGTGGCCGTAG
TCACCTTGAAAAGTGTCGTAAACGTGTTGAAACAAGAAATCGAGAAATTTGGAAACAAATTCGACGA
AATCAAGCTGAAAACATGTCTGCGACAGCTAAAAAGTCTCATAATTCGAAGACCTCTAAGAAGAAAT
TCAAAGAGGACAGAGAATTTGGGACACCAAAAAGATTTTTAAGAGATGATGTTCCTTTCGGGATTGA
TCGTTTGTTTGCTTTTTGATTTTATTTTATATTGTTATCTGTTTCTGTGTATAGACTGTTTGAGATT
GGCGCTTGGCCGACTCATTGTCTTACCATAGGGGAACGGACTTTGTTTGTGTTGTTATTTTATTTGT
ATTTTATTAAAATTCTCAATGATCTGAAAAGGCCTCGAGGCTAAGAGATTATTGGCGGGTGAGTAAG
TACTTTTAAAGTGATGATGGTTACAAAGGCAAAAGGGGTAAAACCCCTCGCCTACGTAAGCGTTATT
ACGCCCGgatcccccggggagctcgaattcgctgaaatcaccagtctctctctacaaatctatctct
ctctattttttccataaataatgtgtgagtagtttcccgataagggaaattagggttcttatagggt
ttcgetcatgtgttgagcatataagaaacccttagtatgtatttgtatttgtaaaatacttctatta
tcaataaaatttctaattcctaaaaccaaaatccagtactaaaatccagatctcctaaagtccctat
agatctttgtcgtgaatataaaccagacacgagacgactaaacdtggagcccagacgccgttcgaag
ctagaagtaccgcttaggcaggaggccgttagggaaaagatgctaaggcagggttggttacgttgac
tcccccgtaggtttggtttaaatatgatgaagtggacggaaggaaggaggaagacaaggaaggataa
ggttgcaggccctgtgcaaggtaagaagatggaaatttgatagaggtacgctactatacttatacta
tacgctaagggaatgcttgtatttataccctataccccctaataaccccttatcaatttaagaaata
atccgcataagcccccgcttaaaaattggtatcagagccatgaataggtctatgaccaaaactcaag
aggataaaacctcaccaaaatacgaaagagttcttaactctaaagataaaagatctttcaagatcaa
aactagttccctcacaccggagcatgcgatatcctcgacctgcaggcatgcaagcttggcgtaatca
tggtcatagctgtttcctgtgtgaaattgttatccgctcacaattccacacaacatacgagccggaa
gcataaagtgtaaagcctggggtgcctaatgagtgagctaactcacattaattgcgttgcgctcact
gcccgctttccagtcgggaaacctgtcgtgccagctgcattaatgaatcggccaacgcgcggggaga
ggcggtttgcgtattgggccaaagacaaaagggcgacattcaaccgattgagggagggaaggtaaat
attgacggaaattattcattaaaggtgaattatcaccgtcaccgacttgagccatttgggaattaga
gccagcaaaatcaccagtagcaccattaccattagcaaggccggaaacgtcaccaatgaaaccatcg
atagcagcaccgtaatcagtagcgacagaatcaagtttgcctttagcgtcagactgtagcgcgtttt
catcggcattttcggtcatagcccccttattagcgtttgccatcttttcataatcaaaatcaccgga
accagagccaccaccggaaccgcctccctcagagccgccaccctcagaaccgccaccctcagagcca
ccaccctcagagccgccaccagaaccaccaccagagccgccgccagcattgacaggaggcccgatct
agtaacatagatgacaccgcgcgcgataatttatcctagtttgcgcgctatattttgttttctatcg
cgtattaaatgtataattgcgggactctaatcataaaaacccatctcataaataacgtcatgcatta
catgttaattattacatgcttaacgtaattcaacagaaattatatgataatcatcgcaagaccggca
acaggattcaatcttaagaaactttattgccaaatgtttgaacgatcggggatcatccgggtctgtg
gcgggaactccacgaaaatatccgaacgcagcaagatatcgcggtgcatctcggtcttgcctgggca
gtcgccgccgacgccgttgatgtggacgccgggcccgatcatattgtcgctcaggatcgtggcgttg
tgcttgtcggccgttgctgtcgtaatgatatcggcaccttcgaccgcctgttccgcagagatcccgt
gggcgaagaactccagcatgagatccccgcgctggaggatcatccagccggcgtcccggaaaacgat
tccgaagcccaacctttcatagaaggcggcggtggaatcgaaatctcgtgatggcaggttgggcgtc
gcttggtcggtcatttcgaaccccagagtcccgctcagaagaactcgtcaagaaggcgatagaaggc
gatgcgctgcgaatcgggagcggcgataccgtaaagcacgaggaagcggtcagcccattcgccgcca
agctcttcagcaatatcacgggtagccaacgctatgtcctgatagcggtccgccacacccagccggc
cacagtcgatgaatccagaaaagcggccattttccaccatgatattcggcaagcaggcatcgccatg
ggtcacgacgagatcatcgccgtcgggcatgcgcgccttgagcctggcgaacagttcggctggcgcg
agcccctgatgctcttcgtccagatcatcctgatcgacaagaccggcttccatccgagtacgtgctc
gctcgatgcgatgtttcgcttggtggtcgaatgggcaggtagccggatcaagcgtatgcagccgccg
cattgcatcagccatgatggatactttctcggcaggagcaaggtgagatgacaggagatcctgcccc
ggcacttcgcccaatagcagccagtcccttcccgcttcagtgacaacgtcgagcacagctgcgcaag
gaacgcccgtcgtggccagccacgatagccgcgctgcctcgtcctgcagttcattcagggcaccgga
caggtcggtcttgacaaaaagaaccgggcgcccctgcgctgacagccggaacacggcggcatcagag
cagccgattgtctgttgtgcccagtcatagccgaatagcctctccacccaagcggccggagaacctg
cgtgcaatccatcttgttcaatcatgcgaaacgatccagatccggtgcagattatttggattgagag
tgaatatgagactctaattggataccgaggggaatttatggaacgtcagtggagcatttttgacaag
aaatatttgctagctgatagtgaccttaggcgacttttgaacgcgcaataatggtttctgacgtatg
tgcttagctcattaaactccagaaacccgcggctgagtggctccttcaacgttgcggttctgtcagt
tccaaacgtaaaacggcttgtcccgcgtcatcggcgggggtcataacgtgactcccttaattctccg
ctcatgatcagattgtcgtttcccgccttcagtttaaactatcagtgtttgacaggatatattggcg
ggtaaacctaagagaaaagagcgtttattagaataatcggatatttaaaagggcgtgaaaaggttta
tccgttcgtccatttgtatgtgcatgccaaccacagggttccccagatctggcgccggccagcgaga
cgagcaagattggccgccgcccgaaacgatccgacagcgcgcccagcacaggtgcgcaggcaaattg
caccaacgcatacagcgccagcagaatgccatagtgggcggtgacgtcgttcgagtgaaccagatcg
cgcaggaggcccggcagcaccggcataatcaggccgatgccgacagcgtcgagcgcgacagtgctca
gaattacgatcaggggtatgttgggtttcacgtctggcctccggaccagcctccgctggtccgattg
aacgcgcggattctttatcactgataagttggtggacatattatgtttatcagtgataaagtgtcaa
gcatgacaaagttgcagccgaatacagtgatccgtgccgccctggacctgttgaacgaggtcggcgt
agacggtctgacgacacgcaaactggcggaacggttgggggttcagcagccggcgctttactggcac
ttcaggaacaagcgggcgctgctcgacgcactggccgaagccatgctggcggagaatcatacgcatt
cggtgccgagagccgacgacgactggcgctcatttctgatcgggaatgcccgcagcttcaggcaggc
gctgctcgcctaccgcgatggcgcgcgcatccatgccggcacgcgaccgggcgcaccgcagatggaa
acggccgacgcgcagcttcgcttcctctgcgaggcgggtttttcggccggggacgccgtcaatgcgc
tgatgacaatcagctacttcactgttggggccgtgcttgaggagcaggccggcgacagcgatgccgg
cgagcgcggcggcaccgttgaacaggctccgctctcgccgctgttgcgggccgcgatagacgccttc
gacgaagccggtccggacgcagcgttcgagcagggactcgcggtgattgtcgatggattggcgaaaa
ggaggctcgttgtcaggaacgttgaaggaccgagaaagggtgacgattgatcaggaccgctgccgga
gcgcaacccactcactacagcagagccatgtagacaacatcccctccccctttccaccgcgtcagac
gcccgtagcagcccgctacgggctttttcatgccctgccctagcgtccaagcctcacggccgcgctc
ggcctctctggcggccttctggcgctcttccgcttcctcgctcactgactcgctgcgctcggtcgtt
cggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgct
ggcgtttttccataggctccgcccccctgacgagcatcacaaaaatcgacgctcaagtcagaggtgg
cgaaacccgacaggactataaagataccaggcgtttccccctggaagctccctcgtgcgctctcctg
ttccgaccctgccgcttaccggatacctgtccgcctttctcccttcgggaagcgtggcgcttttccg
ctgcataaccctgcttcggggtcattatagcgattttttcggtatatccatcctttttcgcacgata
tacaggattttgccaaagggttcgtgtagactttccttggtgtatccaacggcgtcagccgggcagg
ataggtgaagtaggcccacccgcgagcgggtgttccttcttcactgtcccttattcgcacctggcgg
tgctcaacgggaatcctgctctgcgaggctggccggctaccgccggcgtaacagatgagggcaagcg
gatggctgatgaaaccaagccaaccaggaagggcagcccacctatcaaggtgtactgccttccagac
gaacgaagagcgattgaggaaaaggcggcggcggccggcatgagcctgtcggcctacctgctggccg
tcggccagggctacaaaatcacgggcgtcgtggactatgagcacgtccgcgagctggcccgcatcaa
tggcgacctgggccgcctgggcggcctgctgaaactctggctcaccgacgacccgcgcacggcgcgg
ttcggtgatgccacgatcctcgccctgctggcgaagatcgaagagaagcaggacgagcttggcaagg
tcatgatgggcgtggtccgcccgagggcagagccatgacttttttagccgctaaaacggccgggggg
tgcgcgtgattgccaagcacgtccccatgcgctccatcaagaagagcgacttcgcggagctggtgaa
gtacatcaccgacgagcaaggcaagaccgagcgcctttgcgacgctcaccgggctggttgccctcgc
cgctgggctggcggccgtctatggccctgcaaacgcgccagaaacgccgtcgaagccgtgtgcgaga
caccgcggccgccggcgttgtggatacctcgcggaaaacttggccctcactgacagatgaggggcgg
acgttgacacttgaggggccgactcacccggcgcggcgttgacagatgaggggcaggctcgatttcg
gccggcgacgtggagctggccagcctcgcaaatcggcgaaaacgcctgattttacgcgagtttccca
cagatgatgtggacaagcctggggataagtgccctgcggtattgacacttgaggggcgcgactactg
acagatgaggggcgcgatccttgacacttgaggggcagagtgctgacagatgaggggcgcacctatt
gacatttgaggggctgtccacaggcagaaaatccagcatttgcaagggtttccgcccgtttttcggc
caccgctaacctgtcttttaacctgcttttaaaccaatatttataaaccttgtttttaaccagggct
gcgccctgtgcgcgtgaccgcgcacgccgaaggggggtgcccccccttctcgaaccctcccggcccg
ctaacgcgggcctcccatccccccaggggctgcgcccctcggccgcgaacggcctcaccccaaaaat
ggcagcgctggcagtccttgccattgccgggatcggggcagtaacgggatgggcgatcagcccgagc
gcgacgcccggaagcattgacgtgccgcaggtgctggcatcgacattcagcgaccaggtgccgggca
gtgagggcggcggcctgggtggcggcctgcccttcacttcggccgtcggggcattcacggacttcat
ggcggggccggcaatttttaccttgggcattcttggcatagtggtcgcgggtgccgtgctcgtgttc
gggggtgcgataaacccagcgaaccatttgaggtgataggtaagattataccgaggtatgaaaacga
gaattggacctttacagaattactctatgaagcgccatatttaaaaagctaccaagacgaagaggat
gaagaggatgaggaggcagattgccttgaatatattgacaatactgataagataatatatcttttat
atagaagatatcgccgtatgtaaggatttcagggggcaaggcataggcagcgcgcttatcaatatat
ctatagaatgggcaaagcataaaaacttgcatggactaatgcttgaaacccaggacaataaccttat
agcttgtaaattctatcataattgggtaatgactccaacttattgatagtgttttatgttcagataa
tgcccgatgactttgtcatgcagctccaccgattttgagaacgacagcgacttccgtcccagccgtg
ccaggtgctgcctcagattcaggttatgccgctcaattcgctgcgtatatcgcttgctgattacgtg
cagctttcccttcaggcgggattcatacagcggccagccatccgtcatccatatcaccacgtcaaag
ggtgacagcaggctcataagacgccccagcgtcgccatagtgcgttcaccgaatacgtgcgcaacaa
ccgtcttccggagactgtcatacgcgtaaaacagccagcgctggcgcgatttagccccgacatagcc
ccactgttcgtccatttccgcgcagacgatgacgtcactgcccggctgtatgcgcgaggttaccgac
tgcggcctgagttttttaagtgacgtaaaatcgtgttgaggccaacgcccataatgcgggctgttgc
ccggcatccaacgccattcatggccatatcaatgattttctggtgcgtaccgggttgagaagcggtg
taagtgaactgcagttgccatgttttacggcagtgagagcagagatagcgctgatgtccggcggtgc
ttttgccgttacgcaccaccccgtcagtagctgaacaggagggacagctgatagacacagaagccac
tggagcacctcaaaaacaccatcatacactaaatcagtaagttggcagcatcacccataattgtggt
ttcaaaatcggctccgtcgatactatgttatacgccaactttgaaaacaactttgaaaaagctgttt
tctggtatttaaggttttagaatgcaaggaacagtgaattggagttcgtcttgttataattagcttc
ttggggtatctttaaatactgtagaaaagaggaaggaaataataaatggctaaaatgagaatatcac
cggaattgaaaaaactgatcgaaaaataccgctgcgtaaaagatacggaaggaatgtctcctgctaa
ggtatataagctggtgggagaaaatgaaaacctatatttaaaaatgacggacagccggtataaaggg
accacctatgatgtggaacgggaaaaggacatgatgctatggctggaaggaaagctgcctgttccaa
aggtcctgcactttgaacggcatgatggctggagcaatctgctcatgagtgaggccgatggcgtcct
ttgctcggaagagtatgaagatgaacaaagccctgaaaagattatcgagctgtatgcggagtgcatc
aggctctttcactccatcgacatatcggattgtccctatacgaatagcttagacagccgcttagccg
aattggattacttactgaataacgatctggccgatgtggattgcgaaaactgggaagaagacactcc
atttaaagatccgcgcgagctgtatgattttttaaagacggaaaagcccgaagaggaacttgtcttt
tcccacggcgacctgggagacagcaacatctttgtgaaagatggcaaagtaagtggctttattgatc
ttgggagaagcggcagggcggacaagtggtatgacattgccttctgcgtccggtcgatcagggagga
tatcggggaagaacagtatgtcgagctattttttgacttactggggatcaagcctgattgggagaaa
ataaaatattatattttactggatgaattgttttagtacctagatgtggcgcaacgatgccggcgac
aagcaggagcgcaccgacttcttccgcatcaagtgttttggctctcaggccgaggcccacggcaagt
atttgggcaaggggtcgctggtattcgtgcagggcaagattcggaataccaagtacgagaaggacgg
ccagacggtctacgggaccgacttcattgccgataaggtggattatctggacaccaaggcaccaggc
gggtcaaatcaggaataagggcacattgccccggcgtgagtcggggcaatcccgcaaggagggtgaa
tgaatcggacgtttgaccggaaggcatacaggcaagaactgatcgacgcggggttttccgccgagga
tgccgaaaccatcgcaagccgcaccgtcatgcgtgcgccccgcgaaaccttccagtccgtcggctcg
atggtccagcaagctacggccaagatcgagcgcgacagcgtgcaactggctccccctgccctgcccg
cgccatcggccgccgtggagcgttcgcgtcgtctcgaacaggaggcggcaggtttggcgaagtcgat
gaccatcgacacgcgaggaactatgacgaccaagaagcgaaaaaccgccggcgaggacctggcaaaa
caggtcagcgaggccaagcaggccgcgttgctgaaacacacgaagcagcagatcaaggaaatgcagc
tttccttgttcgatattgcgccgtggccggacacgatgcgagcgatgccaaacgacacggcccgctc
tgccctgttcaccacgcgcaacaagaaaatcccgcgcgaggcgctgcaaaacaaggtcattttccac
gtcaacaaggacgtgaagatcacctacaccggcgtcgagctgcgggccgacgatgacgaactggtgt
ggcagcaggtgttggagtacgcgaagcgcacccctatcggcgagccgatcaccttcacgttctacga
gctttgccaggacctgggctggtcgatcaatggccggtattacacgaaggccgaggaatgcctgtcg
cgcctacaggcgacggcgatgggcttcacgtccgaccgcgttgggcacctggaatcggtgtcgctgc
tgcaccgcttccgcgtcctggaccgtggcaagaaaacgtcccgttgccaggtcctgatcgacgagga
aatcgtcgtgctgtttgctggcgaccactacacgaaattcatatgggagaagtaccgcaagctgtcg
ccgacggcccgacggatgttcgactatttcagctcgcaccgggagccgtacccgctcaagctggaaa
ccttccgcctcatgtgcggatcggattccacccgcgtgaagaagtggcgcgagcaggtcggcgaagc
ctgcgaagagttgcgaggcagcggcctggtggaacacgcctgggtcaatgatgacctggtgcattgc
aaacgctagggccttgtggggtcagttccggctgggggttcagcagccagcgctttactggcatttc
aggaacaagcgggcactgctcgacgcacttgcttcgctcagtatcgctcgggacgcacggcgcgctc
tacgaactgccgataaacagaggattaaaattgacaattgtgattaaggctcagattcgacggcttg
gagcggccgacgtgcaggatttccgcgagatccgattgtcggccctgaagaaagctccagagatgtt
cgggtccgtttacgagcacgaggagaaaaagcccatggaggcgttcgctgaacggttgcgagatgcc
gtggcattcggcgcctacatcgacggcgagatcattgggctgtcggtcttcaaacaggaggacggcc
ccaaggacgctcacaaggcgcatctgtccggcgttttcgtggagcccgaacagcgaggccgaggggt
cgccggtatgctgctgcgggcgttgccggcgggtttattgctcgtgatgatcgtccgacagattcca
acgggaatctggtggatgcgcatcttcatcctcggcgcacttaatatttcgctattctggagcttgt
tgtttatttcggtctaccgcctgccgggcggggtcgcggcgacggtaggcgctgtgcagccgctgat
ggtcgtgttcatctctgccgctctgctaggtagcccgatacgattgatggcggtcctgggggctatt
tgcggaactgcgggcgtggcgctgttggtgttgacaccaaacgcagcgctagatcctgtcggcgtcg
cagcgggcctggcgggggcggtttccatggcgttcggaaccgtgctgacccgcaagtggcaacctcc
cgtgcctctgctcacctttaccgcctggcaactggcggccggaggacttctgctcgttccagtagct
ttagtgtttgatccgccaatcccgatgcctacaggaaccaatgttctcggcctggcgtggctcggcc
tgatcggagcgggtttaacctacttcctttggttccgggggatctcgcgactcgaacctacagttgt
ttccttactgggctttctcagccccagatctggggtcgatcagccggggatgcatcaggccgacagt
cggaacttcgggtccccgacctgtaccattcggtgagcaatggataggggagttgatatcgtcaacg
ttcacttctaaagaaatagcgccactcagcttcctcagcggctttatccagcgatttcctattatgt
cggcatagttctcaagatcgacagcctgtcacggttaagcgagaaatgaataagaaggctgataatt
cggatctctgcgagggagatgatatttgatcacaggcagcaacgctctgtcatcgttacaatcaaca
tgctaccctccgcgagatcatccgtgtttcaaacccggcagcttagttgccgttcttccgaatagca
tcggtaacatgagcaaagtctgccgccttacaacggctctcccgctgacgccgtcccggactgatgg
gctgcctgtatcgagtggtgattttgtgccgagctgccggtcggggagctgttggctggctggtggc
aggatatattgtggtgtaaacaaattgacgcttagacaacttaataacacattgcggacgtttttaa
tgtactggggtggtttttcttttcaccagtgagacgggcaacagctgattgcccttcaccgcctggc
cctgagagagttgcagcaagcggtccacgctggtttgccccagcaggcgaaaatcctgtttgatggt
ggttccgaaatcggcaaaatcccttataaatcaaaagaatagcccgagatagggttgagtgttgttc
cagtttggaacaagagtccactattaaagaacgtggactccaacgtcaaagggcgaaaaaccgtcta
tcagggcgatggcccactacgtgaaccatcacccaaatcaagttttttggggtcgaggtgccgtaaa
gcactaaatcggaaccctaaagggagcccccgatttagagcttgacggggaaagccggcgaacgtgg
cgagaaaggaagggaagaaagcgaaaggagcgggcgccattcaggctgcgcaactgttgggaagggc
gatcggtgcgggcctcttcgctattacgccagctggcgaaagggggatgtgctgcaaggcgattaag
ttgggtaacgccagggttttcccagtcacgacgttgtaaaacgacggccagtgaattcgagctcggt
acccccc

### Amplicon Constructs

SEQ ID NO 2 - pBTAΔMP
tactccaaaaatgtcaaagatacagtctcagaagaccaaagggctattgagacttttcaacaaaggg
taatttcgggaaacctcctcggattccattgcccagctatctgtcacttcatcgaaaggacagtaga
aaaggaaggtggctcctacaaatgccatcattgcgataaaggaaaggctatcattcaagatgcctct
gccgacagtggtcccaaagatggacccccacccacgaggagcatcgtggaaaaagaagacgtcccaa
ccacgtcttcaaagcaagtggattgatgtgacatctccactgacgtaagggatgacgcacaatccca
ctatccttcgcaagacccttcttctatataaggaagttcatttcatttggagaggacagcccaagct
ttctagagGATCCATAAAACATTTCAATCCTTTGAACGCGGTAGAACGTGCTAATTGGATTTTGGTG
AGAACGCGGTAGAACGTACTTATCACCTACAGTTTTATTTTGTTTTTCTTTTTGGTTTAATCTATCC
AGCTTAGTACCGAGTGGGGGAAAGTGACTGGTGTGCCTAAAACCTTTTCTTTGATACTTTGTAAAAA
TACATACAGATACAATGGCGAACGGTAACTTCAAGTTGTCTCAATTGCTCAATGTGGACGAGATGTC
TGCTGAGCAGAGGAGTCATTTCTTTGACTTGATGCTGACTAAACCTGATTGTGAGATCGGGCAAATG
ATGCAAAGAGTTGTTGTTGATAAAGTCGATGACATGATTAGAGAAAGAAAGACTAAAGATCCAGTGA
TTGTTCATGAAGTTCTTTCTCAGAAGGAACAGAACAAGTTGATGGAAATTTATCCTGAATTCAATAT
CGTGTTTAAAGACGACAAAAACATGGTTCATGGGTTTGCGGCTGCTGAGCGAAAACTACAAGCTTTA
TTGCTTTTAGATAGAGTTCCTGCTCTGCAAGAGGTGGATGACATCGGTGGTCAATGGTCGTTTTGGG
TAACTAGAGGTGAGAAAAGGATTCATTCCTGTTGTCCAAATCTAGATATTCGGGATGATCAGAGAGA
AATTTCTCGACAGATATTTCTTACTGCTATTGGTGATCAAGCTAGAAGTGGTAAGAGACAGATGTCG
GAGAATGAGCTGTGGATGTATGACCAATTTCGTGAAAATATTGCTGCGCCTAACGCGGTTAGGTGCA
ATAATACATATCAGGGTTGTACATGTAGGGGTTTTTCTGATGGTAAGAAGAAAGGCGCGCAGTATGC
GATAGCTCTTCACAGCCTGTATGACTTCAAGTTGAAAGACTTGATGGCTACTATGGTTGAGAAGAAA
ACTAAAGTGGTTCATGCTGCTATGCTTTTTGCTCCTGAAAGTATGTTAGTGGACGAAGGTCCATTAC
CTTCTGTTGACGGTTACTACATGAAGAAGAACGGGAAGATCTATTTCGGTTTTGAGAAAGATCCTTC
CTTTTCTTACATTCATGACTGGGAAGAGTACAAGAAGTATCTACTGGGGAAGCCAGTGAGTTACCAA
GGGAATGTGTTCTACTTCGAACCGTGGCAGGTGAGAGGAGACACAATGCTTTTTTCGATCTACAGGA
TAGCTGGAGTTCCGAGGAGGTCTCTATCATCGCAAGAGTACTACCGAAGAATATATATCAGTAGATG
GGAAAGCATGGTTGTTGTCCCAATTTTCGATCTGGTCGAATCAACGCGAGAGTTGGTCAAGAAAGAC
CTGTTTGTAGAGAAACAATTCATGGACAAGTGTTTGGATTACATAGCTAGGTTATCTGACCAGCAGC
TGACCATAAGCAATGTTAAATCATACTTGAGTTCAAATAATTGGGTCTTATTCATAAACGGGGCGGC
CGTGAAGAACAAGCAAAGTGTAGATTCTCGAGATTTACAGTTGTTGGCTCAAACTTTGCTAGTGAAG
GAACAAGTGGCGAGACCTGTCATGAGGGAGTTGCGTGAAGCAATTCTGACTGAGACGAAACCTATCA
CGTCATTGACTGATGTGCTGGGTTTAATATCAAGAAAACTGTGGAAGCAGTTTGCTAACAAGATCGC
AGTCGGCGGATTCGTTGGCATGGTTGGTACTCTAATTGGATTCTATCCAAAGAAGGTACTAACCTGG
GCGAAGGACACACCAAATGGTCCAGAACTATGTTACGAGAACTCGCACAAAACCAAGGTGATAGTAT
TTCTGAGTGTTGTGTATGCCATTGGAGGAATCACGCTTATGCGTCGAGACATCCGAGATGGACTGGT
GAAAAAACTATGTGATATGTTTGATATCAAACGGGGGGCCCATGTCTTAGACGTTGAGAATCCGTGC
CGCTATTATGAAATCAACGATTTCTTTAGCAGTCTGTATTCGGCATCTGAGTCCGGTGAGACCGTTT
TACCAGATTTATCCGAGGTAAAAGCCAAGTCTGATAAGCTATTGCAGCAGAAGAAAGAAATCGCTGA
CGAGTTTCTAAGTGCAAAATTCTCTAACTATTCTGGCAGTTCGGTGAGAACTTCTCCACCATCGGTG
GTCGGTTCATCTCGAAGCGGACTGGGTCTGTTGTTGGAAGACAGTAACGTGCTGACCCAAGCTAGAG
TTGGAGTTTCAAGAAAGGTAGACGATGAGGAGATCATGGAGCAGTTTCTGAGTGGTCTTATTGACAC
TGAAGCAGAAATTGACGAGGTTGTTTCAGCCTTTTCAGCTGAATGTGAAAGAGGGGAAACAAGCGGT
ACAAAGGTGTTGTGTAAACCTTTAACGCCACCAGGATTTGAGAACGTGTTGCCAGCTGTCAAACCTT
TGGTCAGCAAAGGAAAAACGGTCAAACGTGTCGATTACTTCCAAGTGATGGGAGGTGAGAGATTACC
AAAAAGGCCGGTTGTCAGTGGAGACGATTCTGTGGACGCTAGAAGAGAGTTTCTGTACTACTTAGAT
GCGGAGAGAGTCGCTCAAAATGATGAAATTATGTCTCTGTATCGTGACTATTCGAGAGGAGTTATTC
GAACTGGAGGTCAGAATTACCCGCACGGACTGGGAGTGTGGGATGTGGAGATGAAGAACTGGTGCAT
ACGTCCAGTGGTCACTGAACATGCTTATGTGTTCCAACCAGACAAACGTATGGATGATTGGTCGGGA
TACTTAGAAGTGGCTGTTTGGGAACGAGGTATGTTGGTCAACGACTTCGCGGTCGAAAGGATGAGTG
ATTATGTCATAGTTTGCGATCAGACGTATCTTTGCAATAACAGGTAATAATCCTCTCTCTTGATATT
TTTAAATTATAGAATTAATTAGTTTACTTTATTCTTTACTATATGATTTAAATAGTTTAATCTTGTT
TTTGAGTAAACTATTCGATTTTGATATTTGTATTCGTCCTACAAAGTTGGAAATACTGATGATATTT
TCTTTTGAACGTGATACCTACCAATACTAATCTTACGGAATCTTTTAATAGAGCACTAATCAACATG
GAACTAAAGACCAATTCTTAAGTGTCTCTGTTGTACAGTTCATTTTAGTAGTGCGTTTAAGTATTAT
TATCTCCCTTCATGCGGGGCAATTATGTAGATTAAAATCGAAATTATATAAAATTTACATAAGTCTA
AGTCTAGGGTCTCCAGCTAATTGTTATTTTTTTAACGATGTTGACTAAAGCAATAACGACGTTGACT
TGTGTTAAACAGGTTGATCTTGGACAATTTAAGTGCCCTGGATCTAGGACCAGTTAACTGTTCTTTT
GAATTAGTTGACGGTGTACCTGGTTGTGGTAAGTCGACAATGATTGTCAACTCAGCTAATCCTTGTG
TCGATGTGGTTCTCTCTACTGGGAGAGCAGCAACCGACGACTTGATCGAGAGATTCGCGAGCAAAGG
TTTTCCATGCAAATTGAAAAGGAGAGTGAAGACGGTTGATTCTTTTTTGATGCATTGTGTCGATGGT
TCTTTAACCGGAGACGTGTTGCATTTCGACGAAGCTCTCATGGCCCATGCTGGTATGGTGTACTTTT
GCGCTCAGATAGCTGGTGCTAAACGATGTATCTGTCAAGGAGATCAGAATCAAATTTCTTTCAAGCC
TAGGGTATCTCAAGTTGATTTGAGGTTTTCTAGTCTGGTCGGAAAGTTTGACATTGTTACAGAAAAA
AGAGAAACTTACAGAAGTCCAGCAGATGTGGCTGCCGTATTGAACAAGTACTATACTGGAGATGTCA
GAACACATAACGCGACTGCTAATTCGATGACGGTGAGGAAGATTGTGTCTAAAGAACAGGTTTCTTT
GAAGCCTGGTGCTCAGTACATAACTTTCCTTCAGTCTGAGAAGAAGGAGTTGGTAAATTTGTTGGCA
TTGAGGAAAGTGGCAGCTAAAGTGAGTACAGTACACGAGTCGCAAGGAGAGACATTCAAAGATGTAG
TCCTAGTCAGGACGAAACCTACGGATGACTCAATCGCTAGAGGTCGGGAGTACTTAATCGTGGCATT
GTCGCGTCACACACAATCACTTGTGTATGAAACTGTGAAAGAGGACGATGTAAGCAAAGAGATCAGG
GAAAGTGCCGCGCTTACGAAGGCGGCTTTGGCAAGATTTTTTGTTACTGAGACCGTCTTATGACGGT
TTCGGTCTAGGTTTGATGTCTTTAGACATCATGAAGGGCCTTGCGCCGTTCCAGATTCAGGTACGAT
TACGGACTTGGAGATGTGGTACGACGCTTTGTTTCCGGGAAATTCGTTAAGAGACTCAAGCCTAGAC
GGGTATTTGGTGGCAACGACTGATTGCAATTTGCGATTAGACAATGTTACGATCAAAAGTGGAAACT
GGAAAGACAAGTTTGCTGAAAAAGAAACGTTTCTGAAACCGGTTATTCGTACTGCTATGCCTGACAA
AAGGAAGACTACTCAGTTGGAGAGTTTGTTAGCATTGCAGAAAAGGAACCAAGCGGCACCCGATCTA
CAAGAAAATGTGCACGCGACAGTTCTAATCGAAGAGACGATGAAGAAGCTGAAATCTGTTGTCTACG
ATGTGGGAAAAATTCGGGCTGATCCTATTGTCAATAGAGCTCAAATGGAGAGATGGTGGAGAAATCA
AAGCACAGCGGTACAGGCTAAGGTAGTAGCAGATGTGAGAGAGTTACATGAAATAGACTATTCGTCT
TACATGTATATGATCAAATCTGACGTGAAACCTAAGACTGATTTAACACCGCAATTTGAATACTCAG
CTCTACAGACTGTTGTGTATCACGAGAAGTTGATCAACTCGTTGTTCGGTCCAATTTTCAAAGAAAT
TAATGAACGCAAGTTGGATGCTATGCAACCACATTTTGTGTTCAACACGAGAATGACATCGAGTGAT
TTAAACGATCGAGTGAAGTTCTTAAATACGGAAGCGGCTTACGACTTTGTTGAGATAGACATGTCTA
AATTCGACAAGTCGGCAAATCGCTTCCATTTACAACTGCAGCTGGAGATTTACAGGTTATTTGGGCT
GGATGAGTGGGCGGCCTTCCTTTGGGAGGTGTCGCACACTCAAACTACTGTGAGAGATATTCAAAAT
GGTATGATGGCGCATATTTGGTACCAACAAAAGAGTGGAGATGCTGATACTTATAATGCAAATTCAG
ATAGAACACTGTGTGCACTCTTGTCTGAATTACCATTGGAGAAAGCAGTCATGGTTACATATGGAGG
AGATGACTCACTGATTGCGTTTCCTAGAGGAACGCAGTTTGTTGATCCGTGTCCAAAGTTGGCTACT
AAGTGGAATTTCGAGTGCAAGATTTTTAAGTACGATGTCCCAATGTTTTGTGGGAAGTTCTTGCTTA
AGACGTCATCGTGTTACGAGTTCGTGCCAGATCCGGTAAAAGTTCTGACGAAGTTGGGGAAAAAGAG
TATAAAGGATGTGCAACATTTAGCCGAGATCTACATCTCGCTGAATGATTCCAATAGAGCTCTTGGG
AACTACATGGTGGTATCCAAACTGTCCGAGTCTGTTTCAGACCGGTATTTGTACAAAGGTGATTCTG
TTCATGCGCTTTGTGCGCTATGGAAGCATATTAAGAGTTTTACAGCTCTGTGTACATTATTCCGAGA
CGAAAACGATAAGGAATTGAACCCGGCTAAGGTTGATTGGAAGAAGGCACAGAGAGCTGTGTCAAAC
TTTTACGACTGGTAATATGGAAGACAAGTCATTGGTCACCTTGAAGAAGAAGACTGGCGCGCCACGT
GTTAATTAACTGATTCGACTAGGCGCCTCAATGTGGAAGAACTGAACAGTTCGGATTACATTGAAGG
CGATTTTACCGATCAAGAGGTTTTCGGTGAGTTCATGTCTTTGAAACAAGTGGAGATGAAGACGATT
GAGGCGAAGTACGATGGTCCTTACAGACCAGCTACTACTAGACCTAAGTCATTATTGTCAAGTGAAG
ATGTTAAGAGAGCGTCTAATAAGAAAAACTCGTCTTAATGCATAAAGAAATTTATTGTCAATATGAC
GTGTGTACTCAAGGGTTGTGTGAATGAAGTCACTGTTCTTGGTCACGAGACGTGTAGTATCGGTCAT
GCTAACAAATTGCGAAAGCAAGTTGCTGACATGGTTGGTGTCACACGTAGGTGTGCGGAAAATAATT
GTGGATGGTTTGTCTGTGTTGTTATCAATGATTTTACTTTTGATGTGTATAATTGTTGTGGCCGTAG
TCACCTTGAAAAGTGTCGTAAACGTGTTGAAACAAGAAATCGAGAAATTTGGAAACAAATTCGACGA
AATCAAGCTGAAAACATGTCTGCGACAGCTAAAAAGTCTCATAATTCGAAGACCTCTAAGAAGAAAT
TCAAAGAGGACAGAGAATTTGGGACACCAAAAAGATTTTTAAGAGATGATGTTCCTTTCGGGATTGA
TCGTTTGTTTGCTTTTTGATTTTATTTTATATTGTTATCTGTTTCTGTGTATAGACTGTTTGAGATT
GGCGCTTGGCCGACTCATTGTCTTACCATAGGGGAACGGACTTTGTTTGTGTTGTTATTTTATTTGT
ATTTTATTAAAATTCTCAATGATCTGAAAAGGCCTCGAGGCTAAGAGATTATTGGGGGGTGAGTAAG
TACTTTTAAAGTGATGATGGTTACAAAGGCAAAAGGGGTAAAACCCCTCGCCTACGTAAGCGTTATT
ACGCCCGgatcccccggggagctcgaattcgctgaaatcaccagtctctctctacaaatctatctct
ctctattttttccataaataatgtgtgagtagtttcccgataagggaaattagggttcttatagggt
ttcgctcatgtgttgagcatataagaaacccttagtatgtatttgtatttgtaaaatacttctatta
tcaataaaatttctaattcctaaaaccaaaatccagtactaaaatccagatctcctaaagtccctat
agatctttgtcgtgaatataaaccagacacgagacgactaaacctggagcccagacgccgttcgaag
ctagaagtaccgcttaggcaggaggccgttagggaaaagatgctaaggcagggttggttacgttgac
tcccccgtaggtttggtttaaatatgatgaagtggacggaaggaaggaggaagacaaggaaggataa
ggttgcaggccctgtgcaaggtaagaagatggaaatttgatagaggtacgctactatacttatacta
tacgctaagggaatgcttgtatttataccctatacaccotaataaccccttatcaatttaagaaata
atccgcataagcccccgcttaaaaattggtatcagagccatgaataggtctatgaccaaaactcaag
aggataaaacctcaccaaaatacgaaagagttcttaactctaaagataaaagatctttcaagatcaa
aactagttccctcacaccggagcatgcgatatcctcgacctgcaggcatgcaagcttggcgtaatca
tggtcatagctgtttcctgtgtgaaattgttatccgctcacaattccacacaacatacgagccggaa
gcataaagtgtaaagcctggggtgcctaatgagtgagctaactcacattaattgcgttgcgctcact
gcccgctttccagtcgggaaacctgtcgtgccagctgcattaatgaatcggccaacgcgcggggaga
ggcggtttgcgtattgggccaaagacaaaagggcgacattcaaccgattgagggagggaaggtaaat
attgacggaaattattcattaaaggtgaattatcaccgtcaccgacttgagccatttgggaattaga
gccagcaaaatcaccagtagcaccattaccattagcaaggccggaaacgtcaccaatgaaaccatcg
atagcagcaccgtaatcagtagcgacagaatcaagtttgcctttagcgtcagactgtagcgcgtttt
catcggcattttcggtcatagcccccttattagcgtttgccatcttttcataatcaaaatcaccgga
accagagccaccaccggaaccgcctccctcagagccgccaccctcagaaccgccaccctcagagcca
ccaccctcagagccgccaccagaaccaccaccagagccgccgccagcattgacaggaggcccgatct
agtaacatagatgacaccgcgcgcgataatttatcctagtttgcgcgctatattttgttttctatcg
cgtattaaatgtataattgcgggactctaatcataaaaacccatctcataaataacgtcatgcatta
catgttaattattacatgcttaacgtaattcaacagaaattatatgataatcatcgcaagaccggca
acaggattcaatcttaagaaactttattgccaaatgtttgaacgatcggggatcatccgggtctgtg
gcgggaactccacgaaaatatccgaacgcagcaagatatcgcggtgcatctcggtcttgcctgggca
gtcgccgccgacgccgttgatgtggacgccgggcccgatcatattgtcgctcaggatcgtggcgttg
tgcttgtcggccgttgctgtcgtaatgatatcggcaccttcgaccgcctgttccgcagagatcccgt
gggcgaagaactccagcatgagatccccgcgctggaggatcatccagccggcgtcccggaaaacgat
tccgaagcccaacctttcatagaaggcggcggtggaatcgaaatctcgtgatggcaggttgggcgtc
gcttggtcggtcatttcgaaccccagagtcccgctcagaagaactcgtcaagaaggcgatagaaggc
gatgcgctgcgaatcgggagcggcgataccgtaaagcacgaggaagcggtcagcccattcgccgcca
agctcttcagcaatatcacgggtagccaacgctatgtcctgatagcggtccgccacacccagccggc
cacagtcgatgaatccagaaaagcggccattttccaccatgatattcggcaagcaggcatcgccatg
ggtcacgacgagatcatcgccgtcgggcatgcgcgccttgagcctggcgaacagttcggctggcgcg
agcccctgatgctcttcgtccagatcatcctgatcgacaagaccggcttccatccgagtacgtgctc
gctcgatgcgatgtttcgcttggtggtcgaatgggcaggtagccggatcaagcgtatgcagccgccg
cattgcatcagccatgatggatactttctcggcaggagcaaggtgagatgacaggagatcctgcccc
ggcacttcgcccaatagcagccagtcccttcccgcttcagtgacaacgtcgagcacagctgcgcaag
gaacgcccgtcgtggccagccacgatagccgcgctgcctcgtcctgcagttcattcagggcaccgga
caggtcggtcttgacaaaaagaaccgggcgcccctgcgctgacagccggaacacggcggcatcagag
cagccgattgtctgttgtgcccagtcatagccgaatagcctctccacccaagcggccggagaacctg
cgtgcaatccatcttgttcaatcatgcgaaacgatccagatccggtgcagattatttggattgagag
tgaatatgagactctaattggataccgaggggaatttatggaacgtcagtggagcatttttgacaag
aaatatttgctagctgatagtgaccttaggcgacttttgaacgcgcaataatggtttctgacgtatg
tgcttagctcattaaactccagaaacccgcggctgagtggctccttcaacgttgcggttctgtcagt
tccaaacgtaaaacggcttgtcccgcgtcatcggcgggggtcataacgtgactcccttaattctccg
ctcatgatcagattgtcgtttcccgccttcagtttaaactatcagtgtttgacaggatatattggcg
ggtaaacctaagagaaaagagcgtttattagaataatcggatatttaaaagggcgtgaaaaggttta
tccgttcgtccatttgtatgtgcatgccaaccacagggttccccagatctggcgccggccagcgaga
cgagcaagattggccgccgcccgaaacgatccgacagcgcgcccagcacaggtgcgcaggcaaattg
caccaacgcatacagcgccagcagaatgccatagtgggcggtgacgtcgttcgagtgaaccagatcg
cgcaggaggcccggcagcaccggcataatcaggccgatgccgacagcgtcgagcgcgacagtgctca
gaattacgatcaggggtatgttgggtttcacgtctggcctccggaccagcctccgctggtccgattg
aacgcgcggattctttatcactgataagttggtggacatattatgtttatcagtgataaagtgtcaa
gcatgacaaagttgcagccgaatacagtgatccgtgccgccctggacctgttgaacgaggtcggcgt
agacggtctgacgacacgcaaactggcggaacggttgggggttcagcagccggcgctttactggcac
ttcaggaacaagcgggcgctgctcgacgcactggccgaagccatgctggcggagaatcatacgcatt
cggtgccgagagccgacgacgactggcgctcatttctgatcgggaatgcccgcagcttcaggcaggc
gctgctcgcctaccgcgatggcgcgcgcatccatgccggcacgcgaccgggcgcaccgcagatggaa
acggccgacgcgcagcttcgcttcctctgcgaggcgggtttttcggccggggacgccgtcaatgcgc
tgatgacaatcagctacttcactgttggggccgtgcttgaggagcaggccggcgacagcgatgccgg
cgagcgcggcggcaccgttgaacaggctccgctctcgccgctgttgcgggccgcgatagacgccttc
gacgaagccggtccggacgcagcgttcgagcagggactcgcggtgattgtcgatggattggcgaaaa
ggaggctcgttgtcaggaacgttgaaggaccgagaaagggtgacgattgatcaggaccgctgccgga
gcgcaacccactcactacagcagagccatgtagacaacatcccctccccctttccaccgcgtcagac
gcccgtagcagcccgctacgggctttttcatgccctgccctagcgtccaagcctcacggccgcgctc
ggcctctctggcggccttctggcgctcttccgcttcctcgctcactgactcgctgcgctcggtcgtt
cggctgcggcgagcggtatcagctcactcaaaggcggtaatacggttatccacagaatcaggggata
acgcaggaaagaacatgtgagcaaaaggccagcaaaaggccaggaaccgtaaaaaggccgcgttgct
ggcgtttttccataggctccgcccccctgacgagcatcacaaaaatcgacgctcaagtcagaggtgg
cgaaacccgacaggactataaagataccaggcgtttccccctggaagctccctcgtgcgctctcctg
ttccgaccctgccgcttaccggatacctgtccgcctttctcccttcgggaagcgtggcgcttttccg
ctgcataaccctgcttcggggtcattatagcgattttttcggtatatccatcctttttcgcacgata
tacaggattttgccaaagggttcgtgtagactttccttggtgtatccaacggcgtcagccgggcagg
ataggtgaagtaggcccacccgcgagcgggtgttccttcttcactgtcccttattcgcacctggcgg
tgctcaacgggaatcctgctctgcgaggctggccggctaccgccggcgtaacagatgagggcaagcg
gatggctgatgaaaccaagccaaccaggaagggcagcccacctatcaaggtgtactgccttccagac
gaacgaagagcgattgaggaaaaggcggcggcggccggcatgagcctgtcggcctacctgctggccg
tcggccagggctacaaaatcacgggcgtcgtggactatgagcacgtccgcgagctggcccgcatcaa
tggcgacctgggccgcctgggcggcctgctgaaactctggctcaccgacgacccgcgcacggcgcgg
ttcggtgatgccacgatcctcgccctgctggcgaagatcgaagagaagcaggacgagcttggcaagg
tcatgatgggcgtggtccgcccgagggcagagccatgacttttttagccgctaaaacggccgggggg
tgcgcgtgattgccaagcacgtccccatgcgctccatcaagaagagcgacttcgcggagctggtgaa
gtacatcaccgacgagcaaggcaagaccgagcgcctttgcgacgctcaccgggctggttgccctcgc
cgctgggctggcggccgtctatggccctgcaaacgcgccagaaacgccgtcgaagccgtgtgcgaga
caccgcggccgccggcgttgtggatacctcgcggaaaacttggccctcactgacagatgaggggcgg
acgttgacacttgaggggccgactcacccggcgcggcgttgacagatgaggggcaggctcgatttcg
gccggcgacgtggagctggccagcctcgcaaatcggcgaaaacgcctgattttacgcgagtttccca
cagatgatgtggacaagcctggggataagtgccctgcggtattgacacttgaggggcgcgactactg
acagatgaggggcgcgatccttgacacttgaggggcagagtgctgacagatgaggggcgcacctatt
gacatttgaggggctgtccacaggcagaaaatccagcatttgcaagggtttccgcccgtttttcggc
caccgctaacctgtcttttaacctgcttttaaaccaatatttataaaccttgtttttaaccagggct
gcgccctgtgcgcgtgaccgcgcacgccgaaggggggtgcccccccttctcgaaccctcccggcccg
ctaacgcgggcctcccatccccccaggggctgcgcccctcggccgcgaacggcctcaccccaaaaat
ggcagcgctggcagtccttgccattgccgggatcggggcagtaacgggatgggcgatcagcccgagc
gcgacgcccggaagcattgacgtgccgcaggtgctggcatcgacattcagcgaccaggtgccgggca
gtgagggcggcggcctgggtggcggcctgcccttcacttcggccgtcggggcattcacggacttcat
ggcggggccggcaatttttaccttgggcattcttggcatagtggtcgcgggtgccgtgctcgtgttc
gggggtgcgataaacccagcgaaccatttgaggtgataggtaagattataccgaggtatgaaaacga
gaattggacctttacagaattactctatgaagcgccatatttaaaaagctaccaagacgaagaggat
gaagaggatgaggaggcagattgccttgaatatattgacaatactgataagataatatatcttttat
atagaagatatcgccgtatgtaaggatttcagggggcaaggcataggcagcgcgcttatcaatatat
ctatagaatgggcaaagcataaaaacttgcatggactaatgcttgaaacccaggacaataaccttat
agcttgtaaattctatcataattgggtaatgactccaacttattgatagtgttttatgttcagataa
tgcccgatgactttgtcatgcagctccaccgattttgagaacgacagcgacttccgtcccagccgtg
ccaggtgctgcctcagattcaggttatgccgctcaattcgctgcgtatatcgcttgctgattacgtg
cagctttcccttcaggcgggattcatacagcggccagccatccgtcatccatatcaccacgtcaaag
ggtgacagcaggctcataagacgccccagcgtcgccatagtgcgttcaccgaatacgtgcgcaacaa
ccgtcttccggagactgtcatacgcgtaaaacagccagcgctggcgcgatttagccccgacatagcc
ccactgttcgtccatttccgcgcagacgatgacgtcactgcccggctgtatgcgcgaggttaccgac
tgcggcctgagttttttaagtgacgtaaaatcgtgttgaggccaacgcccataatgcgggctgttgc
ccggcatccaacgccattcatggccatatcaatgattttctggtgcgtaccgggttgagaagcggtg
taagtgaactgcagttgccatgttttacggcagtgagagcagagatagcgctgatgtccggcggtgc
ttttgccgttacgcaccaccccgtcagtagctgaacaggagggacagctgatagacacagaagccac
tggagcacctcaaaaacaccatcatacactaaatcagtaagttggcagcatcacccataattgtggt
ttcaaaatcggctccgtcgatactatgttatacgccaactttgaaaacaactttgaaaaagctgttt
tctggtatttaaggttttagaatgcaaggaacagtgaattggagttcgtcttgttataattagcttc
ttggggtatctttaaatactgtagaaaagaggaaggaaataataaatggctaaaatgagaatatcac
cggaattgaaaaaactgatcgaaaaataccgctgcgtaaaagatacggaaggaatgtctcctgctaa
ggtatataagctggtgggagaaaatgaaaacctatatttaaaaatgacggacagccggtataaaggg
accacctatgatgtggaacgggaaaaggacatgatgctatggctggaaggaaagctgcctgttccaa
aggtcctgcactttgaacggcatgatggctggagcaatctgctcatgagtgaggccgatggcgtcct
ttgctcggaagagtatgaagatgaacaaagccctgaaaagattatcgagctgtatgcggagtgcatc
aggctctttcactccatcgacatatcggattgtccctatacgaatagcttagacagccgcttagccg
aattggattacttactgaataacgatctggccgatgtggattgcgaaaactgggaagaagacactcc
atttaaagatccgcgcgagctgtatgattttttaaagacggaaaagcccgaagaggaacttgtcttt
tcccacggcgacctgggagacagcaacatctttgtgaaagatggcaaagtaagtggctttattgatc
ttgggagaagcggcagggcggacaagtggtatgacattgccttctgcgtccggtcgatcagggagga
tatcggggaagaacagtatgtcgagctattttttgacttactggggatcaagcctgattgggagaaa
ataaaatattatattttactggatgaattgttttagtacctagatgtggcgcaacgatgccggcgac
aagcaggagcgcaccgacttcttccgcatcaagtgttttggctctcaggccgaggcccacggcaagt
atttgggcaaggggtcgctggtattcgtgcagggcaagattcggaataccaagtacgagaaggacgg
ccagacggtctacgggaccgacttcattgccgataaggtggattatctggacaccaaggcaccaggc
gggtcaaatcaggaataagggcacattgccccggcgtgagtcggggcaatcccgcaaggagggtgaa
tgaatcggacgtttgaccggaaggcatacaggcaagaactgatcgacgcggggttttccgccgagga
tgccgaaaccatcgcaagccgcaccgtcatgcgtgcgccccgcgaaaccttccagtccgtcggctcg
atggtccagcaagctacggccaagatcgagcgcgacagcgtgcaactggctccccctgccctgcccg
cgccatcggccgccgtggagcgttcgcgtcgtctcgaacaggaggcggcaggtttggcgaagtcgat
gaccatcgacacgcgaggaactatgacgaccaagaagcgaaaaaccgccggcgaggacctggcaaaa
caggtcagcgaggccaagcaggccgcgttgctgaaacacacgaagcagcagatcaaggaaatgcagc
tttccttgttcgatattgcgccgtggccggacacgatgcgagcgatgccaaacgacacggcccgctc
tgccctgttcaccacgcgcaacaagaaaatcccgcgcgaggcgctgcaaaacaaggtcattttccac
gtcaacaaggacgtgaagatcacctacaccggcgtcgagctgcgggccgacgatgacgaactggtgt
ggcagcaggtgttggagtacgcgaagcgcacccctatcggcgagccgatcaccttcacgttctacga
gctttgccaggacctgggctggtcgatcaatggccggtattacacgaaggccgaggaatgcctgtcg
cgcctacaggcgacggcgatgggcttcacgtccgaccgcgttgggcacctggaatcggtgtcgctgc
tgcaccgcttccgcgtcctggaccgtggcaagaaaacgtcccgttgccaggtcctgatcgacgagga
aatcgtcgtgctgtttgctggcgaccactacacgaaattcatatgggagaagtaccgcaagctgtcg
ccgacggcccgacggatgttcgactatttcagctcgcaccgggagccgtacccgctcaagctggaaa
ccttccgcctcatgtgcggatcggattccacccgcgtgaagaagtggcgcgagcaggtcggcgaagc
ctgcgaagagttgcgaggcagcggcctggtggaacacgcctgggtcaatgatgacctggtgcattgc
aaacgctagggccttgtggggtcagttccggctgggggttcagcagccagcgctttactggcatttc
aggaacaagcgggcactgctcgacgcacttgcttcgctcagtatcgctcgggacgcacggcgcgctc
tacgaactgccgataaacagaggattaaaattgacaattgtgattaaggctcagattcgacggcttg
gagcggccgacgtgcaggatttccgcgagatccgattgtcggccctgaagaaagctccagagatgtt
cgggtccgtttacgagcacgaggagaaaaagcccatggaggcgttcgctgaacggttgcgagatgcc
gtggcattcggcgcctacatcgacggcgagatcattgggctgtcggtcttcaaacaggaggacggcc
ccaaggacgctcacaaggcgcatctgtccggcgttttcgtggagcccgaacagcgaggccgaggggt
cgccggtatgctgctgcgggcgttgccggcgggtttattgctcgtgatgatcgtccgacagattcca
acgggaatctggtggatgcgcatcttcatcctcggcgcacttaatatttcgctattctggagcttgt
tgtttatttcggtctaccgcctgccgggcggggtcgcggcgacggtaggcgctgtgcagccgctgat
ggtcgtgttcatctctgccgctctgctaggtagcccgatacgattgatggcggtcctgggggctatt
tgcggaactgcgggcgtggcgctgttggtgttgacaccaaacgcagcgctagatcctgtcggcgtcg
cagcgggcctggcgggggcggtttccatggcgttcggaaccgtgctgacccgcaagtggcaacctcc
cgtgcctctgctcacctttaccgcctggcaactggcggccggaggacttctgctcgttccagtagct
ttagtgtttgatccgccaatcccgatgcctacaggaaccaatgttctcggcctggcgtggctcggcc
tgatcggagcgggtttaacctacttcctttggttccgggggatctcgcgactcgaacctacagttgt
ttccttactgggctttctcagccccagatctggggtcgatcagccggggatgcatcaggccgacagt
cggaacttcgggtccccgacctgtaccattcggtgagcaatggataggggagttgatatcgtcaacg
ttcacttctaaagaaatagcgccactcagcttcctcagcggctttatccagcgatttcctattatgt
cggcatagttctcaagatcgacagcctgtcacggttaagcgagaaatgaataagaaggctgataatt
cggatctctgcgagggagatgatatttgatcacaggcagcaacgctctgtcatcgttacaatcaaca
tgctaccctccgcgagatcatccgtgtttcaaacccggcagcttagttgccgttcttccgaatagca
tcggtaacatgagcaaagtctgccgccttacaacggctctcccgctgacgccgtcccggactgatgg
gctgcctgtatcgagtggtgattttgtgccgagctgccggtcggggagctgttggctggctggtggc
aggatatattgtggtgtaaacaaattgacgcttagacaacttaataacacattgcggacgtttttaa
tgtactggggtggtttttcttttcaccagtgagacgggcaacagctgattgcccttcaccgcctggc
cctgagagagttgcagcaagcggtccacgctggtttgccccagcaggcgaaaatcctgtttgatggt
ggttccgaaatcggcaaaatcccttataaatcaaaagaatagcccgagatagggttgagtgttgttc
cagtttggaacaagagtccactattaaagaacgtggactccaacgtcaaagggcgaaaaaccgtcta
tcagggcgatggcccactacgtgaaccatcacccaaatcaagttttttggggtcgaggtgccgtaaa
gcactaaatcggaaccctaaagggagcccccgatttagagcttgacggggaaagccggcgaacgtgg
cgagaaaggaagggaagaaagcgaaaggagcgggcgccattcaggctgcgcaactgttgggaagggc
gatcggtgcgggcctcttcgctattacgccagctggcgaaagggggatgtgctgcaaggcgattaag
ttgggtaacgccagggttttcccagtcacgacgttgtaaaacgacggccagtgaattcgagctcggt
acccccc
SEQ ID NO 3 - pBTAΔMPΔ16K
GATCCATAAAACATTTCAATCCTTTGAACGCGGTAGAACGTGCTAATTGGATTTTGGTGAGAACGCG
GTAGAACGTACTTATCACCTACAGTTTTATTTTGTTTTTCTTTTTGGTTTAATCTATCCAGCTTAGT
ACCGAGTGGGGGAAAGTGACTGGTGTGCCTAAAACCTTTTCTTTGATACTTTGTAAAAATACATACA
GATACAATGGCGAACGGTAACTTCAAGTTGTCTCAATTGCTCAATGTGGACGAGATGTCTGCTGAGC
AGAGGAGTCATTTCTTTGACTTGATGCTGACTAAACCTGATTGTGAGATCGGGCAAATGATGCAAAG
AGTTGTTGTTGATAAAGTCGATGACATGATTAGAGAAAGAAAGACTAAAGATCCAGTGATTGTTCAT
GAAGTTCTTTCTCAGAAGGAACAGAACAAGTTGATGGAAATTTATCCTGAATTCAATATCGTGTTTA
AAGACGACAAAAACATGGTTCATGGGTTTGCGGCTGCTGAGCGAAAACTACAAGCTTTATTGCTTTT
AGATAGAGTTCCTGCTCTGCAAGAGGTGGATGACATCGGTGGTCAATGGTCGTTTTGGGTAACTAGA
GGTGAGAAAAGGATTCATTCCTGTTGTCCAAATCTAGATATTCGGGATGATCAGAGAGAAATTTCTC
GACAGATATTTCTTACTGCTATTGGTGATCAAGCTAGAAGTGGTAAGAGACAGATGTCGGAGAATGA
GCTGTGGATGTATGACCAATTTCGTGAAAATATTGCTGCGCCTAACGCGGTTAGGTGCAATAATACA
TATCAGGGTTGTACATGTAGGGGTTTTTCTGATGGTAAGAAGAAAGGCGCGCAGTATGCGATAGCTC
TTCACAGCCTGTATGACTTCAAGTTGAAAGACTTGATGGCTACTATGGTTGAGAAGAAAACTAAAGT
GGTTCATGCTGCTATGCTTTTTGCTCCTGAAAGTATGTTAGTGGACGAAGGTCCATTACCTTCTGTT
GACGGTTACTACATGAAGAAGAACGGGAAGATCTATTTCGGTTTTGAGAAAGATCCTTCCTTTTCTT
ACATTCATGACTGGGAAGAGTACAAGAAGTATCTACTGGGGAAGCCAGTGAGTTACCAAGGGAATGT
GTTCTACTTCGAACCGTGGCAGGTGAGAGGAGACACAATGCTTTTTTCGATCTACAGGATAGCTGGA
GTTCCGAGGAGGTCTCTATCATCGCAAGAGTACTACCGAAGAATATATATCAGTAGATGGGAAAGCA
TGGTTGTTGTCCCAATTTTCGATCTGGTCGAATCAACGCGAGAGTTGGTCAAGAAAGACCTGTTTGT
AGAGAAACAATTCATGGACAAGTGTTTGGATTACATAGCTAGGTTATCTGACCAGCAGCTGACCATA
AGCAATGTTAAATCATACTTGAGTTCAAATAATTGGGTCTTATTCATAAACGGGGCGGCCGTGAAGA
ACAAGCAAAGTGTAGATTCTCGAGATTTACAGTTGTTGGCTCAAACTTTGCTAGTGAAGGAACAAGT
GGCGAGACCTGTCATGAGGGAGTTGCGTGAAGCAATTCTGACTGAGACGAAACCTATCACGTCATTG
ACTGATGTGCTGGGTTTAATATCAAGAAAACTGTGGAAGCAGTTTGCTAACAAGATCGCAGTCGGCG
GATTCGTTGGCATGGTTGGTACTCTAATTGGATTCTATCCAAAGAAGGTACTAACCTGGGCGAAGGA
CACACCAAATGGTCCAGAACTATGTTACGAGAACTCGCACAAAACCAAGGTGATAGTATTTCTGAGT
GTTGTGTATGCCATTGGAGGAATCACGCTTATGCGTCGAGACATCCGAGATGGACTGGTGAAAAAAC
TATGTGATATGTTTGATATCAAACGGGGGGCCCATGTCTTAGACGTTGAGAATCCGTGCCGCTATTA
TGAAATCAACGATTTCTTTAGCAGTCTGTATTCGGCATCTGAGTCCGGTGAGACCGTTTTACCAGAT
TTATCCGAGGTAAAAGCCAAGTCTGATAAGCTATTGCAGCAGAAGAAAGAAATCGCTGACGAGTTTC
TAAGTGCAAAATTCTCTAACTATTCTGGCAGTTCGGTGAGAACTTCTCCACCATCGGTGGTCGGTTC
ATCTCGAAGCGGACTGGGTCTGTTGTTGGAAGACAGTAACGTGCTGACCCAAGCTAGAGTTGGAGTT
TCAAGAAAGGTAGACGATGAGGAGATCATGGAGCAGTTTCTGAGTGGTCTTATTGACACTGAAGCAG
AAATTGACGAGGTTGTTTCAGCCTTTTCAGCTGAATGTGAAAGAGGGGAAACAAGCGGTACAAAGGT
GTTGTGTAAACCTTTAACGCCACCAGGATTTGAGAACGTGTTGCCAGCTGTCAAACCTTTGGTCAGC
AAAGGAAAAACGGTCAAACGTGTCGATTACTTCCAAGTGATGGGAGGTGAGAGATTACCAAAAAGGC
CGGTTGTCAGTGGAGACGATTCTGTGGACGCTAGAAGAGAGTTTCTGTACTACTTAGATGCGGAGAG
AGTCGCTCAAAATGATGAAATTATGTCTCTGTATCGTGACTATTCGAGAGGAGTTATTCGAACTGGA
GGTCAGAATTACCCGCACGGACTGGGAGTGTGGGATGTGGAGATGAAGAACTGGTGCATACGTCCAG
TGGTCACTGAACATGCTTATGTGTTCCAACCAGACAAACGTATGGATGATTGGTCGGGATACTTAGA
AGTGGCTGTTTGGGAACGAGGTATGTTGGTCAACGACTTCGCGGTCGAAAGGATGAGTGATTATGTC
ATAGTTTGCGATCAGACGTATCTTTGCAATAACAGGTAATAATCCTCTCTCTTGATATTTTTAAATT
ATAGAATTAATTAGTTTACTTTATTCTTTACTATATGATTTAAATAGTTTAATCTTGTTTTTGAGTA
AACTATTCGATTTTGATATTTGTATTCGTCCTACAAAGTTGGAAATACTGATGATATTTTCTTTTGA
ACGTGATACCTACCAATACTAATCTTACGGAATCTTTTAATAGAGCACTAATCAACATGGAACTAAA
GACCAATTCTTAAGTGTCTCTGTTGTACAGTTCATTTTAGTAGTGCGTTTAAGTATTATTATCTCCC
TTCATGCGGGGCAATTATGTAGATTAAAATCGAAATTATATAAAATTTACATAAGTCTAAGTCTAGG
GTCTCCAGCTAATTGTTATTTTTTTAACGATGTTGACTAAAGCAATAACGACGTTGACTTGTGTTAA
ACAGGTTGATCTTGGACAATTTAAGTGCCCTGGATCTAGGACCAGTTAACTGTTCTTTTGAATTAGT
TGACGGTGTACCTGGTTGTGGTAAGTCGACAATGATTGTCAACTCAGCTAATCCTTGTGTCGATGTG
GTTCTCTCTACTGGGAGAGCAGCAACCGACGACTTGATCGAGAGATTCGCGAGCAAAGGTTTTCCAT
GCAAATTGAAAAGGAGAGTGAAGACGGTTGATTCTTTTTTGATGCATTGTGTCGATGGTTCTTTAAC
CGGAGACGTGTTGCATTTCGACGAAGCTCTCATGGCCCATGCTGGTATGGTGTACTTTTGCGCTCAG
ATAGCTGGTGCTAAACGATGTATCTGTCAAGGAGATCAGAATCAAATTTCTTTCAAGCCTAGGGTAT
CTCAAGTTGATTTGAGGTTTTCTAGTCTGGTCGGAAAGTTTGACATTGTTACAGAAAAAAGAGAAAC
TTACAGAAGTCCAGCAGATGTGGCTGCCGTATTGAACAAGTACTATACTGGAGATGTCAGAACACAT
AACGCGACTGCTAATTCGATGACGGTGAGGAAGATTGTGTCTAAAGAACAGGTTTCTTTGAAGCCTG
GTGCTCAGTACATAACTTTCCTTCAGTCTGAGAAGAAGGAGTTGGTAAATTTGTTGGCATTGAGGAA
AGTGGCAGCTAAAGTGAGTACAGTACACGAGTCGCAAGGAGAGACATTCAAAGATGTAGTCCTAGTC
AGGACGAAACCTACGGATGACTCAATCGCTAGAGGTCGGGAGTACTTAATCGTGGCATTGTCGCGTC
ACACACAATCACTTGTGTATGAAACTGTGAAAGAGGACGATGTAAGCAAAGAGATCAGGGAAAGTGC
CGCGCTTACGAAGGCGGCTTTGGCAAGATTTTTTGTTACTGAGACCGTCTTATGACGGTTTCGGTCT
AGGTTTGATGTCTTTAGACATCATGAAGGGCCTTGCGCCGTTCCAGATTCAGGTACGATTACGGACT
TGGAGATGTGGTACGACGCTTTGTTTCCGGGAAATTCGTTAAGAGACTCAAGCCTAGACGGGTATTT
GGTGGCAACGACTGATTGCAATTTGCGATTAGACAATGTTACGATCAAAAGTGGAAACTGGAAAGAC
AAGTTTGCTGAAAAAGAAACGTTTCTGAAACCGGTTATTCGTACTGCTATGCCTGACAAAAGGAAGA
CTACTCAGTTGGAGAGTTTGTTAGCATTGCAGAAAAGGAACCAAGCGGCACCCGATCTACAAGAAAA
TGTGCACGCGACAGTTCTAATCGAAGAGACGATGAAGAAGCTGAAATCTGTTGTCTACGATGTGGGA
AAAATTCGGGCTGATCCTATTGTCAATAGAGCTCAAATGGAGAGATGGTGGAGAAATCAAAGCACAG
CGGTACAGGCTAAGGTAGTAGCAGATGTGAGAGAGTTACATGAAATAGACTATTCGTCTTACATGTA
TATGATCAAATCTGACGTGAAACCTAAGACTGATTTAACACCGCAATTTGAATACTCAGCTCTACAG
ACTGTTGTGTATCACGAGAAGTTGATCAACTCGTTGTTCGGTCCAATTTTCAAAGAAATTAATGAAC
GCAAGTTGGATGCTATGCAACCACATTTTGTGTTCAACACGAGAATGACATCGAGTGATTTAAACGA
TCGAGTGAAGTTCTTAAATACGGAAGCGGCTTACGACTTTGTTGAGATAGACATGTCTAAATTCGAC
AAGTCGGCAAATCGCTTCCATTTACAACTGCAGCTGGAGATTTACAGGTTATTTGGGCTGGATGAGT
GGGCGGCCTTCCTTTGGGAGGTGTCGCACACTCAAACTACTGTGAGAGATATTCAAAATGGTATGAT
GGCGCATATTTGGTACCAACAAAAGAGTGGAGATGCTGATACTTATAATGCAAATTCAGATAGAACA
CTGTGTGCACTCTTGTCTGAATTACCATTGGAGAAAGCAGTCATGGTTACATATGGAGGAGATGACT
CACTGATTGCGTTTCCTAGAGGAACGCAGTTTGTTGATCCGTGTCCAAAGTTGGCTACTAAGTGGAA
TTTCGAGTGCAAGATTTTTAAGTACGATGTCCCAATGTTTTGTGGGAAGTTCTTGCTTAAGACGTCA
TCGTGTTACGAGTTCGTGCCAGATCCGGTAAAAGTTCTGACGAAGTTGGGGAAAAAGAGTATAAAGG
ATGTGCAACATTTAGCCGAGATCTACATCTCGCTGAATGATTCCAATAGAGCTCTTGGGAACTACAT
GGTGGTATCCAAACTGTCCGAGTCTGTTTCAGACCGGTATTTGTACAAAGGTGATTCTGTTCATGCG
CTTTGTGCGCTATGGAAGCATATTAAGAGTTTTACAGCTCTGTGTACATTATTCCGAGACGAAAACG
ATAAGGAATTGAACCCGGCTAAGGTTGATTGGAAGAAGGCACAGAGAGCTGTGTCAAACTTTTACGA
CTGGTAATATGGAAGACAAGTCATTGGTCACCTTGAAGAAGAAGACTGGCGCGCCACGTGTTAATTA
ACTGATTCGACTAGGCGCCTCAATGTGGAAGAACTGAACAGTTCGGATTACATTGAAGGCGATTTTA
CCGATCAAGAGGTTTTCGGTGAGTTCATGTCTTTGAAACAAGTGGAGATGAAGACGATTGAGGCGAA
GTACGATGGTCCTTACAGACCAGCTACTACTAGACCTAAGTCATTATTGTCAAGTGAAGATGTTAAG
AGAGCGTCTAATAAGAAAAACTCGTCTTAATGCATAAAGAAATTTATTGTCAATATGACGTGTGTAC
TCAAGGGTTGTGTGAATGAAGT...GTTCCTTTCGGGATTGATCGTTTGTTTGCTTTTTGATTTTAT
TTTATATTGTTATCTGTTTCTGTGTATAGACTGTTTGAGATTGGCGCTTGGCCGACTCATTGTCTTA
CCATAGGGGAACGGACTTTGTTTGTGTTGTTATTTTATTTGTATTTTATTAAAATTCTCAATGATCT
GAAAAGGCCTCGAGGCTAAGAGATTATTGGGGGGTGAGTAAGTACTTTTAAAGTGATGATGGTTACA
AAGGCAAAAGGGGTAAAACCCCTCGCCTACGTAAGCGTTATTACGCCCG
SEQ ID NO 4 - pBTAΔRepΔMP
GATCCATAAAACATTTCAATCCTTTGAACGCGGTAGAACGTGCTAATTGGATTTTGGTGAGAACGCG
GTAGAACGTACTTATCACCTACAGTTTTATTTTGTTTTTCTTTTTGGTTTAATCTATCCAGCTTAGT
ACCGAGTGGGGGAAAGTGACTGGTGTGCCTAAAACCTTTTCTTTGATACTTTGTAAAAATACATACA
GATACAATGGCGAACGGTAACTTCAAGTTGTCTCAATTGCTCAATGTGGACGAGATGTCTGCTGAGC
AGAGGAGTCATTTCTTTGACTTGATGCTGACTAAACCTGATTGTGAGATCGGGCAAATGATGCAAAG
AGTTGTTGTTGATAAAGTCGATGACATGATTAGAGAAAGAAAGACTAAAGATCCAGTGATTGTTCAT
GAAGTTCTTTCTCAGAAGGAACAGAACAAGTTGATGGAAATTTATCCTGAATTCAATATCGTGTTTA
AAGACGACAAAAACATGGTTCATGGGTTTGCGGCTGCTGAGCGAAAACTACAAGCTTTATTGCTTTT
AGATAGAGTTCCTGCTCTGCAAGAGGTGGATGACATCGGTGGTCAATGGTCGTTTTGGGTAACTAGA
GGTGAGAAAAGGATTCATTCCTGTTGTCCAAATCTAGATATTCGGGATGATCAGAGAGAAATTTCTC
GACAGATATTTCTTACTGCTATTGGTGATCAAGCTAGAAGTGGTAAGAGACAGATGTCGGAGAATGA
GCTGTGGATGTATGACCAATTTCGTGAAAATATTGCTGCGCCTAACGCGGTTAGGTGCAATAATACA
TATCAGGGTTGTACATGTAGGGGTTTTTCTGATGGTAAGAAGAAAGGCGCGCAGTATGCGATAGCTC
TTCACAGCCTGTATGACTTCAAGTTGAAAGACTTGATGGCTACTATGGTTGAGAAGAAAACTAAAGT
GGTTCATGCTGCTATGCTTTTTGCTCCTGAAAGTATGTTAGTGGACGAAGGTCCATTACCTTCTGTT
GACGGTTACTACATGAAGAAGAACGGGAAGATCTATTTCGGTTTTGAGAAAGATCCTTCCTTTTCTT
ACATTCATGACTGGGAAGAGTACAAGAAGTATCTACTGGGGAAGCCAGTGAGTTACCAAGGGAATGT
GTTCTACTTCGAACCGTGGCAGGTGAGAGGAGACACAATGCTTTTTTCGATCTACAGGATAGCTGGA
GTTCCGAGGAGGTCTCTATCATCGCAAGAGTACTACCGAAGAATATATATCAGTAGATGGGAAAGCA
TGGTTGTTGTCCCAATTTTCGATCTGGTCGAATCAACGCGAGAGTTGGTCAAGAAAGACCTGTTTGT
AGAGAAACAATTCATGGACAAGTGTTTGGATTACATAGCTAGGTTATCTGACCAGCAGCTGACCATA
AGCAATGTTAAATCATACTTGAGTTCAAATAATTGGGTCTTATTCATAAACGGGGCGGCCGTGAAGA
ACAAGCAAAGTGTAGATTCTCGAGATTTACAGTTGTTGGCTCAAACTTTGCTAGTGAAGGAACAAGT
GGCGAGACCTGTCATGAGGGAGTTGCGTGAAGCAATTCTGACTGAGACGAAACCTATCACGTCATTG
ACTGATGTGCTGGGTTTAATATCAAGAAAACTGTGGAAGCAGTTTGCTAACAAGATCGCAGTCGGCG
GATTCGTTGGCATGGTTGGTACTCTAATTGGATTCTATCCAAAGAAGGTACTAACCTGGGCGAAGGA
CACACCAAATGGTCCAGAACTATGTTACGAGAACTCGCACAAAACCAAGGTGATAGTATTTCTGAGT
GTTGTGTATGCCATTGGAGGAATCACGCTTATGCGTCGAGACATCCGAGATGGACTGGTGAAAAAAC
TATGTGATATGTTTGATATCAAACGGGGGGCCCATGTCTTAGACGTTGAGAATCCGTGCCGCTATTA
TGAAATCAACGATTTCTTTAGCAGTCTGTATTCGGCATCTGAGTCCGGTGAGACCGTTTTACCAGAT
TTATCCGAGGTAAAAGCCAAGTCTGATAAGCTATTGCAGCAGAAGAAAGAAATCGCTGACGAGTTTC
TAAGTGCAAAATTCTCTAACTATTCTGGCAGTTCGGTGAGAACTTCTCCACCATCGGTGGTCGGTTC
ATCTCGAAGCGGACTGGGTCTGTTGTTGGAAGACAGTAACGTGCTGACCCAAGCTAGAGTTGGAGTT
TCAAGAAAGGTAGACGATGAGGAGATCATGGAGCAGTTTCTGAGTGGTCTTATTGACACTGAAGCAG
AAATTGACGAGGTTGTTTCAGCCTTTTCAGCTGAATGTGAAAGAGGGGAAACAAGCGGTACAAAGGT
GTTGTGTAAACCTTTAACGCCACCAGGATTTGAGAACGTGTTGCCAGCTGTCAAACCTTTGGTCAGC
AAAGGAAAAACGGTCAAACGTGTCGATTACTTCCAAGTGATGGGAGGTGAGAGATTACCAAAAAGGC
CGGTTGTCAGTGGAGACGATTCTGTGGACGCTAGAAGAGAGTTTCTGTACTACTTAGATGCGGAGAG
AGTCGCTCAAAATGATGAAATTATGTCTCTGTATCGTGACTATTCGAGAGGAGTTATTCGAACTGGA
GGTCAGAATTACCCGCACGGACTGGGAGTGTGGGATGTGGAGATGAAGAACTGGTGCATACGTCCAG
TGGTCACTGAACATGCTTATGTGTTCCAACCAGACAAACGTATGGATGATTGGTCGGGATACTTAGA
AGTGGCTGTTTGGGAACGAGGTATGTTGGTCAACGACTTCGCGGTCGAAAGGATGAGTGATTATGTC
ATAGTTTGCGATCAGACGTATCTTTGCAATAACAGGTAATAATCCTCTCTCTTGATATTTTTAAATT
ATAGAATTAATTAGTTTACTTTATTCTTTACTATATGATTT(...)AACTGTTCTTTTGAATTAGTTG
ACGGTGTACCTGGTTGTGGTAAGTCGACAATGATTGTCAACTCAGCTAATCCTTGTGTCGATGTGGT
TCTCTCTACTGGGAGAGCAGCAACCGACGACTTGATCGAGAGATTCGCGAGCAAAGGTTTTCCATGC
AAATTGAAAAGGAGAGTGAAGACGGTTGATTCTTTTTTGATGCATTGTGTCGATGGTTCTTTAACCG
GAGACGTGTTGCATTTCGACGAAGCTCTCATGGCCCATGCTGGTATGGTGTACTTTTGCGCTCAGAT
AGCTGGTGCTAAACGATGTATCTGTCAAGGAGATCAGAATCAAATTTCTTTCAAGCCTAGGGTATCT
CAAGTTGATTTGAGGTTTTCTAGTCTGGTCGGAAAGTTTGACATTGTTACAGAAAAAAGAGAAACTT
ACAGAAGTCCAGCAGATGTGGCTGCCGTATTGAACAAGTACTATACTGGAGATGTCAGAACACATAA
CGCGACTGCTAATTCGATGACGGTGAGGAAGATTGTGTCTAAAGAACAGGTTTCTTTGAAGCCTGGT
GCTCAGTACATAACTTTCCTTCAGTCTGAGAAGAAGGAGTTGGTAAATTTGTTGGCATTGAGGAAAG
TGGCAGCTAAAGTGAGTACAGTACACGAGTCGCAAGGAGAGACATTCAAAGATGTAGTCCTAGTCAG
GACGAAACCTACGGATGACTCAATCGCTAGAGGTCGGGAGTACTTAATCGTGGCATTGTCGCGTCAC
ACACAATCACTTGTGTATGAAACTGTGAAAGAGGACGATGTAAGCAAAGAGATCAGGGAAAGTGCCG
CGCTTACGAAGGCGGCTTTGGCAAGATTTTTTGTTACTGAGACCGTCTTATGACGGTTTCGGTCTAG
GTTTGATGTCTTTAGACATCATGAAGGGCCTTGCGCCGTTCCAGATTCAGGTACGATTACGGACTTG
GAGATGTGGTACGACGCTTTGTTTCCGGGAAATTCGTTAAGAGACTCAAGCCTAGACGGGTATTTGG
TGGCAACGACTGATTGCAATTTGCGATTAGACAATGTTACGATCAAAAGTGGAAACTGGAAAGACAA
GTTTGCTGAAAAAGAAACGTTTCTGAAACCGGTTATTCGTACTGCTATGCCTGACAAAAGGAAGACT
ACTCAGTTGGAGAGTTTGTTAGCATTGCAGAAAAGGAACCAAGCGGCACCCGATCTACAAGAAAATG
TGCACGCGACAGTTCTAATCGAAGAGACGATGAAGAAGCTGAAATCTGTTGTCTACGATGTGGGAAA
AATTCGGGCTGATCCTATTGTCAATAGAGCTCAAATGGAGAGATGGTGGAGAAATCAAAGCACAGCG
GTACAGGCTAAGGTAGTAGCAGATGTGAGAGAGTTACATGAAATAGACTATTCGTCTTACATGTATA
TGATCAAATCTGACGTGAAACCTAAGACTGATTTAACACCGCAATTTGAATACTCAGCTCTACAGAC
TGTTGTGTATCACGAGAAGTTGATCAACTCGTTGTTCGGTCCAATTTTCAAAGAAATTAATGAACGC
AAGTTGGATGCTATGCAACCACATTTTGTGTTCAACACGAGAATGACATCGAGTGATTTAAACGATC
GAGTGAAGTTCTTAAATACGGAAGCGGCTTACGACTTTGTTGAGATAGACATGTCTAAATTCGACAA
GTCGGCAAATCGCTTCCATTTACAACTGCAGCTGGAGATTTACAGGTTATTTGGGCTGGATGAGTGG
GCGGCCTTCCTTTGGGAGGTGTCGCACACTCAAACTACTGTGAGAGATATTCAAAATGGTATGATGG
CGCATATTTGGTACCAACAAAAGAGTGGAGATGCTGATACTTATAATGCAAATTCAGATAGAACACT
GTGTGCACTCTTGTCTGAATTACCATTGGAGAAAGCAGTCATGGTTACATATGGAGGAGATGACTCA
CTGATTGCGTTTCCTAGAGGAACGCAGTTTGTTGATCCGTGTCCAAAGTTGGCTACTAAGTGGAATT
TCGAGTGCAAGATTTTTAAGTACGATGTCCCAATGTTTTGTGGGAAGTTCTTGCTTAAGACGTCATC
GTGTTACGAGTTCGTGCCAGATCCGGTAAAAGTTCTGACGAAGTTGGGGAAAAAGAGTATAAAGGAT
GTGCAACATTTAGCCGAGATCTACATCTCGCTGAATGATTCCAATAGAGCTCTTGGGAACTACATGG
TGGTATCCAAACTGTCCGAGTCTGTTTCAGACCGGTATTTGTACAAAGGTGATTCTGTTCATGCGCT
TTGTGCGCTATGGAAGCATATTAAGAGTTTTACAGCTCTGTGTACATTATTCCGAGACGAAAACGAT
AAGGAATTGAACCCGGCTAAGGTTGATTGGAAGAAGGCACAGAGAGCTGTGTCAAACTTTTACGACT
GGTAATATGGAAGACAAGTCATTGGTCACCTTGAAGAAGAAGACTGGCGCGCCACGTGTTAATTAAC
TGATTCGACTAGGCGCCTCAATGTGGAAGAACTGAACAGTTCGGATTACATTGAAGGCGATTTTACC
GATCAAGAGGTTTTCGGTGAGTTCATGTCTTTGAAACAAGTGGAGATGAAGACGATTGAGGCGAAGT
ACGATGGTCCTTACAGACCAGCTACTACTAGACCTAAGTCATTATTGTCAAGTGAAGATGTTAAGAG
AGCGTCTAATAAGAAAAACTCGTCTTAATGCATAAAGAAATTTATTGTCAATATGACGTGTGTACTC
AAGGGTTGTGTGAATGAAGTCACTGTTCTTGGTCACGAGACGTGTAGTATCGGTCATGCTAACAAAT
TGCGAAAGCAAGTTGCTGACATGGTTGGTGTCACACGTAGGTGTGCGGAAAATAATTGTGGATGGTT
TGTCTGTGTTGTTATCAATGATTTTACTTTTGATGTGTATAATTGTTGTGGCCGTAGTCACCTTGAA
AAGTGTCGTAAACGTGTTGAAACAAGAAATCGAGAAATTTGGAAACAAATTCGACGAAATCAAGCTG
AAAACATGTCTGCGACAGCTAAAAAGTCTCATAATTCGAAGACCTCTAAGAAGAAATTCAAAGAGGA
CAGAGAATTTGGGACACCAAAAAGATTTTTAAGAGATGATGTTCCTTTCGGGATTGATCGTTTGTTT
GCTTTTTGATTTTATTTTATATTGTTATCTGTTTCTGTGTATAGACTGTTTGAGATTGGCGCTTGGC
CGACTCATTGTCTTACCATAGGGGAACGGACTTTGTTTGTGTTGTTATTTTATTTGTATTTTATTAA
AATTCTCAATGATCTGAAAAGGCCTCGAGGCTAAGAGATTATTGGGGGGTGAGTAAGTACTTTTAAA
GTGATGATGGTTACAAAGGCAAAAGGGGTAAAACCCCTCGCCTACGTAAGCGTTATTACGCCCG
SEQ ID NO 5 - pBTAΔRepΔM A16K
GATCCATAAAACATTTCAATCCTTTGAACGCGGTAGAACGTGCTAATTGGATTTTGGTGAGAACGCG
GTAGAACGTACTTATCACCTACAGTTTTATTTTGTTTTTCTTTTTGGTTTAATCTATCCAGCTTAGT
ACCGAGTGGGGGAAAGTGACTGGTGTGCCTAAAACCTTTTCTTTGATACTTTGTAAAAATACATACA
GATACAATGGCGAACGGTAACTTCAAGTTGTCTCAATTGCTCAATGTGGACGAGATGTCTGCTGAGC
AGAGGAGTCATTTCTTTGACTTGATGCTGACTAAACCTGATTGTGAGATCGGGCAAATGATGCAAAG
AGTTGTTGTTGATAAAGTCGATGACATGATTAGAGAAAGAAAGACTAAAGATCCAGTGATTGTTCAT
GAAGTTCTTTCTCAGAAGGAACAGAACAAGTTGATGGAAATTTATCCTGAATTCAATATCGTGTTTA
AAGACGACAAAAACATGGTTCATGGGTTTGCGGCTGCTGAGCGAAAACTACAAGCTTTATTGCTTTT
AGATAGAGTTCCTGCTCTGCAAGAGGTGGATGACATCGGTGGTCAATGGTCGTTTTGGGTAACTAGA
GGTGAGAAAAGGATTCATTCCTGTTGTCCAAATCTAGATATTCGGGATGATCAGAGAGAAATTTCTC
GACAGATATTTCTTACTGCTATTGGTGATCAAGCTAGAAGTGGTAAGAGACAGATGTCGGAGAATGA
GCTGTGGATGTATGACCAATTTCGTGAAAATATTGCTGCGCCTAACGCGGTTAGGTGCAATAATACA
TATCAGGGTTGTACATGTAGGGGTTTTTCTGATGGTAAGAAGAAAGGCGCGCAGTATGCGATAGCTC
TTCACAGCCTGTATGACTTCAAGTTGAAAGACTTGATGGCTACTATGGTTGAGAAGAAAACTAAAGT
GGTTCATGCTGCTATGCTTTTTGCTCCTGAAAGTATGTTAGTGGACGAAGGTCCATTACCTTCTGTT
GACGGTTACTACATGAAGAAGAACGGGAAGATCTATTTCGGTTTTGAGAAAGATCCTTCCTTTTCTT
ACATTCATGACTGGGAAGAGTACAAGAAGTATCTACTGGGGAAGCCAGTGAGTTACCAAGGGAATGT
GTTCTACTTCGAACCGTGGCAGGTGAGAGGAGACACAATGCTTTTTTCGATCTACAGGATAGCTGGA
GTTCCGAGGAGGTCTCTATCATCGCAAGAGTACTACCGAAGAATATATATCAGTAGATGGGAAAGCA
TGGTTGTTGTCCCAATTTTCGATCTGGTCGAATCAACGCGAGAGTTGGTCAAGAAAGACCTGTTTGT
AGAGAAACAATTCATGGACAAGTGTTTGGATTACATAGCTAGGTTATCTGACCAGCAGCTGACCATA
AGCAATGTTAAATCATACTTGAGTTCAAATAATTGGGTCTTATTCATAAACGGGGCGGCCGTGAAGA
ACAAGCAAAGTGTAGATTCTCGAGATTTACAGTTGTTGGCTCAAACTTTGCTAGTGAAGGAACAAGT
GGCGAGACCTGTCATGAGGGAGTTGCGTGAAGCAATTCTGACTGAGACGAAACCTATCACGTCATTG
ACTGATGTGCTGGGTTTAATATCAAGAAAACTGTGGAAGCAGTTTGCTAACAAGATCGCAGTCGGCG
GATTCGTTGGCATGGTTGGTACTCTAATTGGATTCTATCCAAAGAAGGTACTAACCTGGGCGAAGGA
CACACCAAATGGTCCAGAACTATGTTACGAGAACTCGCACAAAACCAAGGTGATAGTATTTCTGAGT
GTTGTGTATGCCATTGGAGGAATCACGCTTATGCGTCGAGACATCCGAGATGGACTGGTGAAAAAAC
TATGTGATATGTTTGATATCAAACGGGGGGCCCATGTCTTAGACGTTGAGAATCCGTGCCGCTATTA
TGAAATCAACGATTTCTTTAGCAGTCTGTATTCGGCATCTGAGTCCGGTGAGACCGTTTTACCAGAT
TTATCCGAGGTAAAAGCCAAGTCTGATAAGCTATTGCAGCAGAAGAAAGAAATCGCTGACGAGTTTC
TAAGTGCAAAATTCTCTAACTATTCTGGCAGTTCGGTGAGAACTTCTCCACCATCGGTGGTCGGTTC
ATCTCGAAGCGGACTGGGTCTGTTGTTGGAAGACAGTAACGTGCTGACCCAAGCTAGAGTTGGAGTT
TCAAGAAAGGTAGACGATGAGGAGATCATGGAGCAGTTTCTGAGTGGTCTTATTGACACTGAAGCAG
AAATTGACGAGGTTGTTTCAGCCTTTTCAGCTGAATGTGAAAGAGGGGAAACAAGCGGTACAAAGGT
GTTGTGTAAACCTTTAACGCCACCAGGATTTGAGAACGTGTTGCCAGCTGTCAAACCTTTGGTCAGC
AAAGGAAAAACGGTCAAACGTGTCGATTACTTCCAAGTGATGGGAGGTGAGAGATTACCAAAAAGGC
CGGTTGTCAGTGGAGACGATTCTGTGGACGCTAGAAGAGAGTTTCTGTACTACTTAGATGCGGAGAG
AGTCGCTCAAAATGATGAAATTATGTCTCTGTATCGTGACTATTCGAGAGGAGTTATTCGAACTGGA
GGTCAGAATTACCCGCACGGACTGGGAGTGTGGGATGTGGAGATGAAGAACTGGTGCATACGTCCAG
TGGTCACTGAACATGCTTATGTGTTCCAACCAGACAAACGTATGGATGATTGGTCGGGATACTTAGA
AGTGGCTGTTTGGGAACGAGGTATGTTGGTCAACGACTTCGCGGTCGAAAGGATGAGTGATTATGTC
ATAGTTTGCGATCAGACGTATCTTTGCAATAACAGGTAATAATCCTCTCTCTTGATATTTTTAAATT
ATAGAATTAATTAGTTTACTTTATTCTTTACTATATGATTT(...)AACTGTTCTTTTGAATTAGTTG
ACGGTGTACCTGGTTGTGGTAAGTCGACAATGATTGTCAACTCAGCTAATCCTTGTGTCGATGTGGT
TCTCTCTACTGGGAGAGCAGCAACCGACGACTTGATCGAGAGATTCGCGAGCAAAGGTTTTCCATGC
AAATTGAAAAGGAGAGTGAAGACGGTTGATTCTTTTTTGATGCATTGTGTCGATGGTTCTTTAACCG
GAGACGTGTTGCATTTCGACGAAGCTCTCATGGCCCATGCTGGTATGGTGTACTTTTGCGCTCAGAT
AGCTGGTGCTAAACGATGTATCTGTCAAGGAGATCAGAATCAAATTTCTTTCAAGCCTAGGGTATCT
CAAGTTGATTTGAGGTTTTCTAGTCTGGTCGGAAAGTTTGACATTGTTACAGAAAAAAGAGAAACTT
ACAGAAGTCCAGCAGATGTGGCTGCCGTATTGAACAAGTACTATACTGGAGATGTCAGAACACATAA
CGCGACTGCTAATTCGATGACGGTGAGGAAGATTGTGTCTAAAGAACAGGTTTCTTTGAAGCCTGGT
GCTCAGTACATAACTTTCCTTCAGTCTGAGAAGAAGGAGTTGGTAAATTTGTTGGCATTGAGGAAAG
TGGCAGCTAAAGTGAGTACAGTACACGAGTCGCAAGGAGAGACATTCAAAGATGTAGTCCTAGTCAG
GACGAAACCTACGGATGACTCAATCGCTAGAGGTCGGGAGTACTTAATCGTGGCATTGTCGCGTCAC
ACACAATCACTTGTGTATGAAACTGTGAAAGAGGACGATGTAAGCAAAGAGATCAGGGAAAGTGCCG
CGCTTACGAAGGCGGCTTTGGCAAGATTTTTTGTTACTGAGACCGTCTTATGACGGTTTCGGTCTAG
GTTTGATGTCTTTAGACATCATGAAGGGCCTTGCGCCGTTCCAGATTCAGGTACGATTACGGACTTG
GAGATGTGGTACGACGCTTTGTTTCCGGGAAATTCGTTAAGAGACTCAAGCCTAGACGGGTATTTGG
TGGCAACGACTGATTGCAATTTGCGATTAGACAATGTTACGATCAAAAGTGGAAACTGGAAAGACAA
GTTTGCTGAAAAAGAAACGTTTCTGAAACCGGTTATTCGTACTGCTATGCCTGACAAAAGGAAGACT
ACTCAGTTGGAGAGTTTGTTAGCATTGCAGAAAAGGAACCAAGCGGCACCCGATCTACAAGAAAATG
TGCACGCGACAGTTCTAATCGAAGAGACGATGAAGAAGCTGAAATCTGTTGTCTACGATGTGGGAAA
AATTCGGGCTGATCCTATTGTCAATAGAGCTCAAATGGAGAGATGGTGGAGAAATCAAAGCACAGCG
GTACAGGCTAAGGTAGTAGCAGATGTGAGAGAGTTACATGAAATAGACTATTCGTCTTACATGTATA
TGATCAAATCTGACGTGAAACCTAAGACTGATTTAACACCGCAATTTGAATACTCAGCTCTACAGAC
TGTTGTGTATCACGAGAAGTTGATCAACTCGTTGTTCGGTCCAATTTTCAAAGAAATTAATGAACGC
AAGTTGGATGCTATGCAACCACATTTTGTGTTCAACACGAGAATGACATCGAGTGATTTAAACGATC
GAGTGAAGTTCTTAAATACGGAAGCGGCTTACGACTTTGTTGAGATAGACATGTCTAAATTCGACAA
GTCGGCAAATCGCTTCCATTTACAACTGCAGCTGGAGATTTACAGGTTATTTGGGCTGGATGAGTGG
GCGGCCTTCCTTTGGGAGGTGTCGCACACTCAAACTACTGTGAGAGATATTCAAAATGGTATGATGG
CGCATATTTGGTACCAACAAAAGAGTGGAGATGCTGATACTTATAATGCAAATTCAGATAGAACACT
GTGTGCACTCTTGTCTGAATTACCATTGGAGAAAGCAGTCATGGTTACATATGGAGGAGATGACTCA
CTGATTGCGTTTCCTAGAGGAACGCAGTTTGTTGATCCGTGTCCAAAGTTGGCTACTAAGTGGAATT
TCGAGTGCAAGATTTTTAAGTACGATGTCCCAATGTTTTGTGGGAAGTTCTTGCTTAAGACGTCATC
GTGTTACGAGTTCGTGCCAGATCCGGTAAAAGTTCTGACGAAGTTGGGGAAAAAGAGTATAAAGGAT
GTGCAACATTTAGCCGAGATCTACATCTCGCTGAATGATTCCAATAGAGCTCTTGGGAACTACATGG
TGGTATCCAAACTGTCCGAGTCTGTTTCAGACCGGTATTTGTACAAAGGTGATTCTGTTCATGCGCT
TTGTGCGCTATGGAAGCATATTAAGAGTTTTACAGCTCTGTGTACATTATTCCGAGACGAAAACGAT
AAGGAATTGAACCCGGCTAAGGTTGATTGGAAGAAGGCACAGAGAGCTGTGTCAAACTTTTACGACT
GGTAATATGGAAGACAAGTCATTGGTCACCTTGAAGAAGAAGACTGGCGCGCCACGTGTTAATTAAC
TGATTCGACTAGGCGCCTCAATGTGGAAGAACTGAACAGTTCGGATTACATTGAAGGCGATTTTACC
GATCAAGAGGTTTTCGGTGAGTTCATGTCTTTGAAACAAGTGGAGATGAAGACGATTGAGGCGAAGT
ACGATGGTCCTTACAGACCAGCTACTACTAGACCTAAGTCATTATTGTCAAGTGAAGATGTTAAGAG
AGCGTCTAATAAGAAAAACTCGTCTTAATGCATAAAGAAATTTATTGTCAATATGACGTGTGTACTC
AAGGGTTGTGTGAATGAAGT...GTTCCTTTCGGGATTGATCGTTTGTTTGCTTTTTGATTTTATTT
TATATTGTTATCTGTTTCTGTGTATAGACTGTTTGAGATTGGCGCTTGGCCGACTCATTGTCTTACC
ATAGGGGAACGGACTTTGTTTGTGTTGTTATTTTATTTGTATTTTATTAAAATTCTCAATGATCTGA
AAAGGCCTCGAGGCTAAGAGATTATTGGGGGGTGAGTAAGTACTTTTAAAGTGATGATGGTTACAAA
GGCAAAAGGGGTAAAACCCCTCGCCTACGTAAGCGTTATTACGCCCG

### Sequences Inserted Into the Amplicon Constructs

SEQ ID NO 6 - A. thaliana Partial CDNA Sequence Sulphur Gene.
ccttcactctcttctccttcctcaaaaccttcctcctcccccatttgcttcaggccaggtaaattgt
ttggaagcaagttaaatgcaggaatccaaataaggccaaagaagaacaggtctcgttaccatgtttc
ggttatgaatgtagccactgaaatcaactctactgaacaagtagtagggaagtttgattcaaagaag
agtgcgagaccggtttatccatttgcagctatagtagggcaagatgagatgaagttatgtcttttgt
tgaatgttattgatccaaagattggtggtgttatgattatgggagatagaggaactggaaaatctac
aactgttagatcattagttgatctgttacctgagattaatgtagttgcaggtgacccgtataactcg
gatccgatagatcctgagtttatgggtgttgaagtaagagagagagttgagaaaggagagcaagttc
ctgttattgcgactaagattaatatggttgatcttcctttgggtgcaacagaagatagagtttgtgg
aaccatcgatatcgaaaaggctttgacagaaggtgtaaaagcctttgagcctggtttgttggctaaa
gctaatagagggattctttatgttgatgaagttaatctcttggatgatcatttggttgatgttcttt
tggattcagctgcttctggttggaatacggttgagagagaagggatttcgatttctcacccggcgag
gtttatcttgatcggttcaggaaatccggaagaaggagagcttaggccacagcttcttgatcggttt
ggtatgcatgcacaagtagggacggttagagatgctgatttacgggtcaagattgttgaagagagag
ctcgtttcgatagtaacccaaaggatttccgtgacacttacaaaaccgagcaggacaagcttcaaga
ccagatt
SEQ ID NO 7 - A. thaliana Partial cDNA Sequence RUBISCO Small
Sub-Unit Gene.
cctctatgctctcttccgctactatggttgcctctccggctcaggccactatggtcgctcctttcaa
cggacttaagtcctccgctgccttcccagccacccgcaaggctaacaacgacattacttccatcaca
agcaacggcggaagagttaactgcatgcaggtgtggcctccgattggaaagaagaagtttgagactc
tctcttaccttcctgaccttaccgattccgaattggctaaggaagttgactaccttatccgcaacaa
gtggattccttgtgttgaattcgagttggagcacggatttgtgtaccgtgagcacggtaactcaccc
ggatactatgatggacggtactggacaatgtggaagcttcccttgttcggttgcaccgactccgctc
aagtgttgaaggaagtggaagagtgcaagaaggagtaccccaatgccttcattaggatcatcggatt
SEQ ID NO 8 - A. thaliana Partial cDNA Sequence LEAFY Gene.
ccatacggtatacgtttctacacggcggcgaagatagcggagttaggttttacggcgagcacgcttg
tgggtatgaaggacgaggagcttgaagagatgatgaatagtctctctcatatctttcgttgggagct
tcttgttggtgaacggtacggtatcaaagctgccgttagagctgaacggagacgattgcaagaagag
gaggaagaggaatcttctagacgccgtcatttgctactctccgccgctggtgattccggtactcatc
acgctcttgatgctctctcccaagaagtgattggacagggttatctgaggaaccggtgcagcaacaa
gaccagactgatgcggcggggaataacggcggaggaggaagtggttactgggacgcaggtcaaggaa
agatgaagaagcaacagcagcagagacggagaaagaaaccaatgctgacgtcagtggaaaccgacga
agacgtcaacgaaggtgaggatgacgacgggatggataacggcaacggaggtagtggtttggggaca
gagagacagagggagcatccgtttatcgtaacggagcctggggaagtggcacgtggcaaaaagaacg
gcttagattatctgttccacttgtacgaacaatgccgtgagttccttcttcaggtccagacaattgc
taaagaccgtggcgaaaaatgccccaccaaggtgacgaaccaagtattcaggtacgcgaagaaatca
ggagcgagttacataaacaagcctaaaatgcgacactacgttcactgttacgctctccactgcctag
acgaagaagcttcaaatgctctcagaagagcgtttaaagaacgcggtgagaacgttggctcatggcg
tcaggcttgttacaagccacttgtgaacatcgcttgtcgtcatggctgggatatagacgccgtcttt
aa
SEQ ID NO 9 - mGFP5 cDNA Sequence.
aatgaagactaatctttttctctttctcatcttttcacttctcctatcattatcctcggccgaattc
agtaaaggagaagaacttttcactggagttgtcccaattcttgttgaattagatggtgatgttaatg
ggcacaaattttctgtcagtggagagggtgaaggtgatgcaacatacggaaaacttacccttaaatt
tatttgcactactggaaaactacctgttccatggccaacacttgtcactactttctcttatggtgtt
caatgcttttcaagatacccagatcatatgaagcggcacgacttcttcaagagcgccatgcctgagg
gatacgtgcaggagaggaccatcttcttcaaggacgacgggaactacaagacacgtgctgaagtcaa
gtttgagggagacaccctcgtcaacaggatcgagcttaagggaatcgatttcaaggaggacggaaac
atcctcggccacaagttggaatacaactacaactcccacaacgtatacatcatggccgacaagcaga
agaacggcatcaaagccaacttcaagacccgccacaacatcgaagacggcggcgtgcaactcgctga
tcattatcaacaaaatactccaattggcgatggccctgtccttttaccagacaaccattacctgtcc
acacaatctgccctttcgaaagatcccaacgaaaagagagaccacatggtccttcttgagtttgtaa
cagctgctgggattacacatggcatggatgaactatacaaacatgacgaactctaa

1. An insolated DNA vector which comprises:
(a) transfer nucleotide sequence comprising (i) a plant active promoter, operably linked to (ii) a recombinant tobacco rattle virus (TRV) nucleic acid which includes: a sequence encoding a TRV trans acting factor, and cis acting elements, which confer on the TRV nucleic acid transcript the ability to replicate in the cytoplasm of a plant cell; and a heterologous nucleotide sequence which is foreign to said virus;
(b) border sequences which permit the transfer of the transfer nucleotide sequence into a plant cell genome.

2. A vector as claimed in claim 1 wherein the promoter is constitutive.

3. A vector as claimed in claim 1 wherein the promoter is: developmentally regulated, inducible, or tissue specific.

4. A vector as claimed in any one of the preceding claims comprising a transcriptional terminator within the transfer nucleotide sequence.

5. A vector as claimed in any one of the preceding claims wherein the recombinant TRV nucleic acid does not encode a TRV coat protein.

6. A vector as claimed in any one of the preceding claims wherein the recombinant TRV nucleic acid corresponds to all or part of TRV RNA 1.

7. A vector as claimed in any one of the preceding claims wherein the recombinant TRV nucleic acid lacks all or part of the viral genome not required for replication in the cytoplasm.

8. A vector as claimed in claim 6 which does not comprises an MP and\or 16K ORF.

9. A vector as claimed in claim 7 or claim 8 wherein the heterologous nucleotide sequence is replaces part of the viral genome.

10. A vector as claimed in any one of claims 1 to 8 wherein the heterologous nucleotide sequence is additional to the viral genome.

11. A vector as claimed in any one of the preceding claims wherein the heterologous nucleotide sequence does not contain and is operably linked to a subgenomic promoter.

12. A vector as claimed in any one of the preceding claims wherein the heterologous gene sequence is a multiple cloning site.

13. A vector as claimed in claim 12 wherein the multiple cloning site comprises at least AscI, PmlI and PacI restriction enzyme sites.

14. A vector as claimed in any one of claims 1 to 11 wherein the heterologous gene sequence is a targeting sequence which is capable of down-regulating expression of a target gene.

15. A vector as claimed in claim 14 wherein the targeting sequence is, in a sense or anti-sense orientation, either:
(a) complementary to a sequence within the target gene, or
(b) homologous to a sequence within the target gene.

16. A vector as claimed in claim 14 or claim 15 wherein the targeting sequence targets a conserved sequence within a target gene group such as to down-regulate expression of one or more members of a target gene group.

17. A vector as claimed in any one claims 14 to 16 comprising more than one targeting sequence.

18. A vector as claimed in any one of claims 14 to 17 wherein the target gene is: an endogenous plant gene, a transgene, or a gene from a pathogen.

19. A vector as claimed in claim 18 wherein the endogenous plant gene is one associated with one or more of the following traits: ripening, pollen formation, lignin biosynthesis, flower pigment production, regulatory pathways controlling development, environmental responses, growth, disease resistance, toxin production.

20. A vector as claimed in any one of the preceding claims which is an Agrobacterium binary vector.

21. A vector as claimed in claim 20 which is derived from transformation vector pBINTRA6 (SEQ ID NO: 1).

22. A vector as claimed in claim 21 which is pBTAΔMPΔ16K (SEQ ID NO: 3) or pBTAΔMP (SEQ ID NO: 2)

23. A vector as claimed in any one of the preceding claims which is suitable for stable transformation of a plant cell.

24. A host cell containing a vector of any one of the preceding claims.

25. A method which includes the step of causing or allowing transcription from a vector of any one of claims 14 to 23. within a plant cell to produce a cytoplasmically-replicating RNA.

26. A method of silencing a target gene in to at least part of a plant, the method including:
(i) introducing a vector of any one of claims 14 to 23 into at least part of a plant,
(ii) causing or allowing transcription from the vector to produce a cytoplasmically-replicating RNA.

27. A method as claimed in claim 26 wherein the vector is introduced by Agrobacterium-mediated transient transformation.

28. A method of characterizing a target gene comprising the steps of:
(a) silencing the target gene in at least part of a plant according to a method of claim 26 or claim 27,
(b) observing the phenotype of the part of the plant in which the target gene has been silenced.

29. A method as claimed in claim 28 wherein the phenotype is compared with a plant or part of a plant wherein the target gene is being expressed.

30. A method as claimed in any one of claims 26 to 29 wherein the at least part of the plant includes the meristems of the plant.

31. Use of a vector of any one of claims 14 to 23 in the production of a transgenic plant.

32. A method of silencing a target gene in a plant, the method including:
(i) introducing a vector of any one of claims 14 to 23 into a plant cell such that the transfer nucleotide sequence is stably incorporated into the genome of the cell,
(ii) regenerating a plant from the plant cell.

33. A plant cell having incorporated into its genome the transfer nucleotide sequence of any one of claims 14 to 23.

34. A plant obtainable by the process of claim 32 comprising the plant cell of claim 33.

35. A plant which is clone or descendant of the plant of claim 34 and which comprises the plant cell of claim 33.

36. A plant as claimed in claim 34 or claim 35 which is selected from: Arabidopsis thaliana; Allium cepa; Amaranthus caudatus; Amaranthus retroflexus; Antirrhinum majus; snap-dragon; Arachis hypogaea; Avena sativa; Bellis perennis; Beta vulgaris; Brassica campestris; Brassica campestris ssp. napus; Brassica campestris ssp. pekinensis; Brassica juncea; Calendula officinalis; Capsella bursa-pastoris; Capsicum annuum; Catharanthus roseus; Cheiranthus cheiri; Chenopodium album; Chenopodium amaranticolor; Chenopodium foetidum; Chenopodium quinoa; Coriandrum sativum; Cucumis melo; Cucumis sativus; Glycine max; Gomphrena globosa; Gypsophila elegans; Helianthus annuus; Hyacinthus; Hyoscyamus niger; Lactuca sativa; Lathyrus odoratus; Linum usitatissimum; Lobelia erinus; Lupinus mutabilis; Lycopersicon esculentum; Lycopersicon pimpinellifolium; Melilotus albus; Momordica balsamina; Myosotis sylvatica; Narcissus pseudonarcissus; Nicandra physalodes; Nicotiana benthamiana; Nicotiana clevelandii; Nicotiana glutinosa; Nicotiana rustica; Nicotiana sylvestris; Nicotiana tabacum; Nicotiana edwardsonii; Ocimum basilicum; Petunia hybrida; Phaseolus vulgaris; Phytolacca americana; Pisum sativum; Raphanus sativus; Ricinus communis; Salvia splendens; Senecio vulgaris; Solanum melongena; Solanum nigrum; Solanum tuberosum; Spinacia oleracea; Stellaria media; Trifolium pratense; Trifolium repens; Tropaeolum majus; Tulipa; Vicia faba; Vicia villosa; Viola arvensis.

37. A plant propagule of the plant of any of claims 34 to 36 and which comprises the plant cell of claim 33.

38. An extract or derivative of the plant or plant propagule of any one of claims 34 to 37 and which comprises the plant cell of claim 33.

39. A process for producing a vector of claim of any one of claims 14 to 23, which process comprises the step of cloning a targeting sequence into a vector of claim 12 or 13.


SEQUENCE LISTING (32 sequences)

SEQ ID NO: 1 - DNA, 20174 bp, Artificial Sequence
  Description of Artificial Sequence pBINTRA6
tactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgtcccaaccacgtcttcaaagcaagtggattgatgtgacatctccactgacgtaagggatgacgcacaatcccactatccttcgcaagacccttcttctatataaggaagttcatttcatttggagaggacagcccaagctttctagaggatccataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaagcatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgtttcagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggtaataatcctctctcttgatatttttaaattatagaattaattagtttactttattctttactatatgatttaaatagtttaatcttgtttttgagtaaactattcgattttgatatttgtattcgtcctacaaagttggaaatactgatgatattttcttttgaacgtgatacctaccaatactaatcttacggaatcttttaatagagcactaatcaacatggaactaaagaccaattcttaagtgtctctgttgtacagttcattttagtagtgcgtttaagtattattatctcccttcatgcggggcaattatgtagattaaaatcgaaattatataaaatttacataagtctaagtctagggtctccagctaattgttatttttttaacgatgttgactaaagcaataacgacgttgacttgtgttaaacaggttgatcttggacaatttaagtgccctggatctaggaccagttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgtcgatggttctttaaccggagacgtgttgcatttcgacgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcattgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcgacagttctaatcgaagagacgatgaagaagctgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctggatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactttcgaagtctcaaaattctcaaatctaggggccattgaattgtttgtggacggtaggaggaagagaccgaagtattttcacagaagaagagaaactgtcctaaatcatgttggtgggaagaagagtgaacacaagttagacgtttttgaccaaagggattacaaaatgattaaatcttacgcgtttctaaaggtagtaggtgtacaactagttgtaacatcacatctacctgcagatacgcctgggttcattcaaatcgatctgttggattcgagacttactgagaaaagaaagagaggaaagactattcagagattcaaagctcgagcttgcgataactgttcagttgcgcagtacaaggttgaatacagtatttccacacaggagaacgtacttgatgtctggaaggtgggttgtatttctgagggcgttccggtctgtgacggtacataccctttcagtatcgaagtgtcgctaatatgggttgctactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtcactgttcttggtcacgagacgtgtagtatcggtcatgctaacaaattgcgaaagcaagttgctgacatggttggtgtcacacgtaggtgtgcggaaaataattgtggatggtttgtctgtgttgttatcaatgattttacttttgatgtgtataattgttgtggccgtagtcaccttgaaaagtgtcgtaaacgtgttgaaacaagaaatcgagaaatttggaaacaaattcgacgaaatcaagctgaaaacatgtctgcgacagctaaaaagtctcataattcgaagacctctaagaagaaattcaaagaggacagagaatttgggacaccaaaaagatttttaagagatgatgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccggatcccccggggagctcgaattcgctgaaatcaccagtctctctctacaaatctatctctctctattttttccataaataatgtgtgagtagtttcccgataagggaaattagggttcttatagggtttcgctcatgtgttgagcatataagaaacccttagtatgtatttgtatttgtaaaatacttctattatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggagcatgcgatatcctcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccccc

SEQ ID NO: 2 - DNA, 19705 bp, Artificial Sequence
  Description of Artificial Sequence pBTA-delta -MP
tactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgtcccaaccacgtcttcaaagcaagtggattgatgtgacatctccactgacgtaagggatgacgcacaatcccactatccttcgcaagacccttcttctatataaggaagttcatttcatttggagaggacagcccaagctttctagaggatccataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaagcatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgtttcagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggtaataatcctctctcttgatatttttaaattatagaattaattagtttactttattctttactatatgatttaaatagtttaatcttgtttttgagtaaactattcgattttgatatttgtattcgtcctacaaagttggaaatactgatgatattttcttttgaacgtgatacctaccaatactaatcttacggaatcttttaatagagcactaatcaacatggaactaaagaccaattcttaagtgtctctgttgtacagttcattttagtagtgcgtttaagtattattatctcccttcatgcggggcaattatgtagattaaaatcgaaattatataaaatttacataagtctaagtctagggtctccagctaattgttatttttttaacgatgttgactaaagcaataacgacgttgacttgtgttaaacaggttgatcttggacaatttaagtgccctggatctaggaccagttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgtcgatggttctttaaccggagacgtgttgcatttcgacgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcattgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcgacagttctaatcgaagagacgatgaagaagctgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctggatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactggcgcgccacgtgttaattaactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtcactgttcttggtcacgagacgtgtagtatcggtcatgctaacaaattgcgaaagcaagttgctgacatggttggtgtcacacgtaggtgtgcggaaaataattgtggatggtttgtctgtgttgttatcaatgattttacttttgatgtgtataattgttgtggccgtagtcaccttgaaaagtgtcgtaaacgtgttgaaacaagaaatcgagaaatttggaaacaaattcgacgaaatcaagctgaaaacatgtctgcgacagctaaaaagtctcataattcgaagacctctaagaagaaattcaaagaggacagagaatttgggacaccaaaaagatttttaagagatgatgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccggatcccccggggagctcgaattcgctgaaatcaccagtctctctctacaaatctatctctctctattttttccataaataatgtgtgagtagtttcccgataagggaaattagggttcttatagggtttcgctcatgtgttgagcatataagaaacccttagtatgtatttgtatttgtaaaatacttctattatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccggagcatgcgatatcctcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggccaaagacaaaagggcgacattcaaccgattgagggagggaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccatcttttcataatcaaaatcaccggaaccagagccaccaccggaaccgcctccctcagagccgccaccctcagaaccgccaccctcagagccaccaccctcagagccgccaccagaaccaccaccagagccgccgccagcattgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatatcgcggtgcatctcggtcttgcctgggcagtcgccgccgacgccgttgatgtggacgccgggcccgatcatattgtcgctcaggatcgtggcgttgtgcttgtcggccgttgctgtcgtaatgatatcggcaccttcgaccgcctgttccgcagagatcccgtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccccc

SEQ ID NO: 3 - DNA, 6411 bp, Artificial Sequence
  Description of Artificial Sequence pBTA-delta-MP-delta-16K
gatccataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaagcatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgtttcagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggtaataatcctctctcttgatatttttaaattatagaattaattagtttactttattctttactatatgatttaaatagtttaatcttgtttttgagtaaactattcgattttgatatttgtattcgtcctacaaagttggaaatactgatgatattttcttttgaacgtgatacctaccaatactaatcttacggaatcttttaatagagcactaatcaacatggaactaaagaccaattcttaagtgtctctgttgtacagttcattttagtagtgcgtttaagtattattatctcccttcatgcggggcaattatgtagattaaaatcgaaattatataaaatttacataagtctaagtctagggtctccagctaattgttatttttttaacgatgttgactaaagcaataacgacgttgacttgtgttaaacaggttgatcttggacaatttaagtgccctggatctaggaccagttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgtcgatggttctttaaccggagacgtgttgcatttcgacgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcattgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcgacagttctaatcgaagagacgatgaagaagctgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctggatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactggcgcgccacgtgttaattaactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccg

SEQ ID NO: 4 - DNA, 6358 bp, Artificial Sequence
  Description of Artificial Sequence pBTA-delta-REP-delta-MP
gatccataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaagcatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgtttcagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggtaataatcctctctcttgatatttttaaattatagaattaattagtttactttattctttactatatgatttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgtcgatggttctttaaccggagacgtgttgcatttcgacgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcattgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcgacagttctaatcgaagagacgatgaagaagctgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctggatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactggcgcgccacgtgttaattaactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtcactgttcttggtcacgagacgtgtagtatcggtcatgctaacaaattgcgaaagcaagttgctgacatggttggtgtcacacgtaggtgtgcggaaaataattgtggatggtttgtctgtgttgttatcaatgattttacttttgatgtgtataattgttgtggccgtagtcaccttgaaaagtgtcgtaaacgtgttgaaacaagaaatcgagaaatttggaaacaaattcgacgaaatcaagctgaaaacatgtctgcgacagctaaaaagtctcataattcgaagacctctaagaagaaattcaaagaggacagagaatttgggacaccaaaaagatttttaagagatgatgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccg

SEQ ID NO: 5 - DNA, 6003 bp, Artificial Sequence
  Description of Artificial Sequence pBTA-delta-REP-delta-MP-delta-16K
gatccataaaacatttcaatcctttgaacgcggtagaacgtgctaattggattttggtgagaacgcggtagaacgtacttatcacctacagttttattttgtttttctttttggtttaatctatccagcttagtaccgagtgggggaaagtgactggtgtgcctaaaaccttttctttgatactttgtaaaaatacatacagatacaatggcgaacggtaacttcaagttgtctcaattgctcaatgtggacgagatgtctgctgagcagaggagtcatttctttgacttgatgctgactaaacctgattgtgagatcgggcaaatgatgcaaagagttgttgttgataaagtcgatgacatgattagagaaagaaagactaaagatccagtgattgttcatgaagttctttctcagaaggaacagaacaagttgatggaaatttatcctgaattcaatatcgtgtttaaagacgacaaaaacatggttcatgggtttgcggctgctgagcgaaaactacaagctttattgcttttagatagagttcctgctctgcaagaggtggatgacatcggtggtcaatggtcgttttgggtaactagaggtgagaaaaggattcattcctgttgtccaaatctagatattcgggatgatcagagagaaatttctcgacagatatttcttactgctattggtgatcaagctagaagtggtaagagacagatgtcggagaatgagctgtggatgtatgaccaatttcgtgaaaatattgctgcgcctaacgcggttaggtgcaataatacatatcagggttgtacatgtaggggtttttctgatggtaagaagaaaggcgcgcagtatgcgatagctcttcacagcctgtatgacttcaagttgaaagacttgatggctactatggttgagaagaaaactaaagtggttcatgctgctatgctttttgctcctgaaagtatgttagtggacgaaggtccattaccttctgttgacggttactacatgaagaagaacgggaagatctatttcggttttgagaaagatccttccttttcttacattcatgactgggaagagtacaagaagtatctactggggaagccagtgagttaccaagggaatgtgttctacttcgaaccgtggcaggtgagaggagacacaatgcttttttcgatctacaggatagctggagttccgaggaggtctctatcatcgcaagagtactaccgaagaatatatatcagtagatgggaaagcatggttgttgtcccaattttcgatctggtcgaatcaacgcgagagttggtcaagaaagacctgtttgtagagaaacaattcatggacaagtgtttggattacatagctaggttatctgaccagcagctgaccataagcaatgttaaatcatacttgagttcaaataattgggtcttattcataaacggggcggccgtgaagaacaagcaaagtgtagattctcgagatttacagttgttggctcaaactttgctagtgaaggaacaagtggcgagacctgtcatgagggagttgcgtgaagcaattctgactgagacgaaacctatcacgtcattgactgatgtgctgggtttaatatcaagaaaactgtggaagcagtttgctaacaagatcgcagtcggcggattcgttggcatggttggtactctaattggattctatccaaagaaggtactaacctgggcgaaggacacaccaaatggtccagaactatgttacgagaactcgcacaaaaccaaggtgatagtatttctgagtgttgtgtatgccattggaggaatcacgcttatgcgtcgagacatccgagatggactggtgaaaaaactatgtgatatgtttgatatcaaacggggggcccatgtcttagacgttgagaatccgtgccgctattatgaaatcaacgatttctttagcagtctgtattcggcatctgagtccggtgagaccgttttaccagatttatccgaggtaaaagccaagtctgataagctattgcagcagaagaaagaaatcgctgacgagtttctaagtgcaaaattctctaactattctggcagttcggtgagaacttctccaccatcggtggtcggttcatctcgaagcggactgggtctgttgttggaagacagtaacgtgctgacccaagctagagttggagtttcaagaaaggtagacgatgaggagatcatggagcagtttctgagtggtcttattgacactgaagcagaaattgacgaggttgtttcagccttttcagctgaatgtgaaagaggggaaacaagcggtacaaaggtgttgtgtaaacctttaacgccaccaggatttgagaacgtgttgccagctgtcaaacctttggtcagcaaaggaaaaacggtcaaacgtgtcgattacttccaagtgatgggaggtgagagattaccaaaaaggccggttgtcagtggagacgattctgtggacgctagaagagagtttctgtactacttagatgcggagagagtcgctcaaaatgatgaaattatgtctctgtatcgtgactattcgagaggagttattcgaactggaggtcagaattacccgcacggactgggagtgtgggatgtggagatgaagaactggtgcatacgtccagtggtcactgaacatgcttatgtgttccaaccagacaaacgtatggatgattggtcgggatacttagaagtggctgtttgggaacgaggtatgttggtcaacgacttcgcggtcgaaaggatgagtgattatgtcatagtttgcgatcagacgtatctttgcaataacaggtaataatcctctctcttgatatttttaaattatagaattaattagtttactttattctttactatatgatttaactgttcttttgaattagttgacggtgtacctggttgtggtaagtcgacaatgattgtcaactcagctaatccttgtgtcgatgtggttctctctactgggagagcagcaaccgacgacttgatcgagagattcgcgagcaaaggttttccatgcaaattgaaaaggagagtgaagacggttgattcttttttgatgcattgtgtcgatggttctttaaccggagacgtgttgcatttcgacgaagctctcatggcccatgctggtatggtgtacttttgcgctcagatagctggtgctaaacgatgtatctgtcaaggagatcagaatcaaatttctttcaagcctagggtatctcaagttgatttgaggttttctagtctggtcggaaagtttgacattgttacagaaaaaagagaaacttacagaagtccagcagatgtggctgccgtattgaacaagtactatactggagatgtcagaacacataacgcgactgctaattcgatgacggtgaggaagattgtgtctaaagaacaggtttctttgaagcctggtgctcagtacataactttccttcagtctgagaagaaggagttggtaaatttgttggcattgaggaaagtggcagctaaagtgagtacagtacacgagtcgcaaggagagacattcaaagatgtagtcctagtcaggacgaaacctacggatgactcaatcgctagaggtcgggagtacttaatcgtggcattgtcgcgtcacacacaatcacttgtgtatgaaactgtgaaagaggacgatgtaagcaaagagatcagggaaagtgccgcgcttacgaaggcggctttggcaagattttttgttactgagaccgtcttatgacggtttcggtctaggtttgatgtctttagacatcatgaagggccttgcgccgttccagattcaggtacgattacggacttggagatgtggtacgacgctttgtttccgggaaattcgttaagagactcaagcctagacgggtatttggtggcaacgactgattgcaatttgcgattagacaatgttacgatcaaaagtggaaactggaaagacaagtttgctgaaaaagaaacgtttctgaaaccggttattcgtactgctatgcctgacaaaaggaagactactcagttggagagtttgttagcattgcagaaaaggaaccaagcggcacccgatctacaagaaaatgtgcacgcgacagttctaatcgaagagacgatgaagaagctgaaatctgttgtctacgatgtgggaaaaattcgggctgatcctattgtcaatagagctcaaatggagagatggtggagaaatcaaagcacagcggtacaggctaaggtagtagcagatgtgagagagttacatgaaatagactattcgtcttacatgtatatgatcaaatctgacgtgaaacctaagactgatttaacaccgcaatttgaatactcagctctacagactgttgtgtatcacgagaagttgatcaactcgttgttcggtccaattttcaaagaaattaatgaacgcaagttggatgctatgcaaccacattttgtgttcaacacgagaatgacatcgagtgatttaaacgatcgagtgaagttcttaaatacggaagcggcttacgactttgttgagatagacatgtctaaattcgacaagtcggcaaatcgcttccatttacaactgcagctggagatttacaggttatttgggctggatgagtgggcggccttcctttgggaggtgtcgcacactcaaactactgtgagagatattcaaaatggtatgatggcgcatatttggtaccaacaaaagagtggagatgctgatacttataatgcaaattcagatagaacactgtgtgcactcttgtctgaattaccattggagaaagcagtcatggttacatatggaggagatgactcactgattgcgtttcctagaggaacgcagtttgttgatccgtgtccaaagttggctactaagtggaatttcgagtgcaagatttttaagtacgatgtcccaatgttttgtgggaagttcttgcttaagacgtcatcgtgttacgagttcgtgccagatccggtaaaagttctgacgaagttggggaaaaagagtataaaggatgtgcaacatttagccgagatctacatctcgctgaatgattccaatagagctcttgggaactacatggtggtatccaaactgtccgagtctgtttcagaccggtatttgtacaaaggtgattctgttcatgcgctttgtgcgctatggaagcatattaagagttttacagctctgtgtacattattccgagacgaaaacgataaggaattgaacccggctaaggttgattggaagaaggcacagagagctgtgtcaaacttttacgactggtaatatggaagacaagtcattggtcaccttgaagaagaagactggcgcgccacgtgttaattaactgattcgactaggcgcctcaatgtggaagaactgaacagttcggattacattgaaggcgattttaccgatcaagaggttttcggtgagttcatgtctttgaaacaagtggagatgaagacgattgaggcgaagtacgatggtccttacagaccagctactactagacctaagtcattattgtcaagtgaagatgttaagagagcgtctaataagaaaaactcgtcttaatgcataaagaaatttattgtcaatatgacgtgtgtactcaagggttgtgtgaatgaagtgttcctttcgggattgatcgtttgtttgctttttgattttattttatattgttatctgtttctgtgtatagactgtttgagattggcgcttggccgactcattgtcttaccataggggaacggactttgtttgtgttgttattttatttgtattttattaaaattctcaatgatctgaaaaggcctcgaggctaagagattattggggggtgagtaagtacttttaaagtgatgatggttacaaaggcaaaaggggtaaaacccctcgcctacgtaagcgttattacgcccg

SEQ ID NO: 6 - DNA, 945 bp, Artificial Sequence
  Description of Artificial Sequence A. thaliana partial cDNA sequence sulphur gene
ccttcactctcttctccttcctcaaaaccttcctcctcccccatttgcttcaggccaggtaaattgtttggaagcaagttaaatgcaggaatccaaataaggccaaagaagaacaggtctcgttaccatgtttcggttatgaatgtagccactgaaatcaactctactgaacaagtagtagggaagtttgattcaaagaagagtgcgagaccggtttatccatttgcagctatagtagggcaagatgagatgaagttatgtcttttgttgaatgttattgatccaaagattggtggtgttatgattatgggagatagaggaactggaaaatctacaactgttagatcattagttgatctgttacctgagattaatgtagttgcaggtgacccgtataactcggatccgatagatcctgagtttatgggtgttgaagtaagagagagagttgagaaaggagagcaagttcctgttattgcgactaagattaatatggttgatcttcctttgggtgcaacagaagatagagtttgtggaaccatcgatatcgaaaaggctttgacagaaggtgtaaaagcctttgagcctggtttgttggctaaagctaatagagggattctttatgttgatgaagttaatctcttggatgatcatttggttgatgttcttttggattcagctgcttctggttggaatacggttgagagagaagggatttcgatttctcacccggcgaggtttatcttgatcggttcaggaaatccggaagaaggagagcttaggccacagcttcttgatcggtttggtatgcatgcacaagtagggacggttagagatgctgatttacgggtcaagattgttgaagagagagctcgtttcgatagtaacccaaaggatttccgtgacacttacaaaaccgagcaggacaagcttcaagaccagatt

SEQ ID NO: 7 - DNA, 469 bp, Artificial Sequence
  Description of Artificial Sequence A. thaliana partial cDNA sequence RUBISCO small subunit gene
cctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccgaattggctaaggaagttgactaccttatccgcaacaagtggattccttgtgttgaattcgagttggagcacggatttgtgtaccgtgagcacggtaactcacccggatactatgatggacggtactggacaatgtggaagcttcccttgttcggttgcaccgactccgctcaagtgttgaaggaagtggaagagtgcaagaaggagtaccccaatgccttcattaggatcatcggatt

SEQ ID NO: 8 - DNA, 940 bp, Artificial Sequence
  Description of Artificial Sequence A. thaliana partial cDNA sequence LEAFY gene
ccatacggtatacgtttctacacggcggcgaagatagcggagttaggttttacggcgagcacgcttgtgggtatgaaggacgaggagcttgaagagatgatgaatagtctctctcatatctttcgttgggagcttcttgttggtgaacggtacggtatcaaagctgccgttagagctgaacggagacgattgcaagaagaggaggaagaggaatcttctagacgccgtcatttgctactctccgccgctggtgattccggtactcatcacgctcttgatgctctctcccaagaagtgattggacagggttatctgaggaaccggtgcagcaacaagaccagactgatgcggcggggaataacggcggaggaggaagtggttactgggacgcaggtcaaggaaagatgaagaagcaacagcagcagagacggagaaagaaaccaatgctgacgtcagtggaaaccgacgaagacgtcaacgaaggtgaggatgacgacgggatggataacggcaacggaggtagtggtttggggacagagagacagagggagcatccgtttatcgtaacggagcctggggaagtggcacgtggcaaaaagaacggcttagattatctgttccacttgtacgaacaatgccgtgagttccttcttcaggtccagacaattgctaaagaccgtggcgaaaaatgccccaccaaggtgacgaaccaagtattcaggtacgcgaagaaatcaggagcgagttacataaacaagcctaaaatgcgacactacgttcactgttacgctctccactgcctagacgaagaagcttcaaatgctctcagaagagcgtttaaagaacgcggtgagaacgttggctcatggcgtcaggcttgttacaagccacttgtgaacatcgcttgtcgtcatggctgggatatagacgccgtctttaa

SEQ ID NO: 9 - DNA, 793 bp, Artificial Sequence
  Description of Artificial Sequence mGFP5 cDNA sequence
aatgaagactaatctttttctctttctcatcttttcacttctcctatcattatcctcggccgaattcagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcagaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaacatgacgaactctaa

SEQ ID NO: 10 - DNA, 29 bp, Artificial Sequence
  Description of Artificial Sequence Primer
ggggggatccgggcgtaataacgcttacg

SEQ ID NO: 11 - DNA, 31 bp, Artificial Sequence
  Description of Artificial Sequence Primer
ggggggatccataaaacatttcaatcctttg

SEQ ID NO: 12 - DNA, 22 bp, Artificial Sequence
  Description of Artificial Sequence Primer
ttagcaccagctatctgagcgc

SEQ ID NO: 13 - DNA, 23 bp, Artificial Sequence
  Description of Artificial Sequence Primer
gttccaaccagacaaacgtatgg

SEQ ID NO: 14 - DNA, 43 bp, Artificial Sequence
  Description of Artificial Sequence Primer
cgtatctttgcaataacaggtaataatcctctctcttgatatt

SEQ ID NO: 15 - DNA, 42 bp, Artificial Sequence
  Description of Artificial Sequence Primer
ttaaattgtccaagatcaacctgtttaacacaagtcaacgtc

SEQ ID NO: 16 - DNA, 22 bp, Artificial Sequence
  Description of Artificial Sequence Primer
tcgcacaaaaccaaggtgatag

SEQ ID NO: 17 - DNA, 33 bp, Artificial Sequence
  Description of Artificial Sequence Primer
ggattattacctgttattgcaaagatacgtctg

SEQ ID NO: 18 - DNA, 34 bp, Artificial Sequence
  Description of Artificial Sequence Primer
tgttaaacaggttgatcttggacaatttaagtgc

SEQ ID NO: 19 - DNA, 22 bp, Artificial Sequence
  Description of Artificial Sequence Primer
ttctcaaatctaggggccattg

SEQ ID NO: 20 - DNA, 33 bp, Artificial Sequence
  Description of Artificial Sequence Primer
ccgaaaggaacacttcattcacacaacccttga

SEQ ID NO: 21 - DNA, 31 bp, Artificial Sequence
  Description of Artificial Sequence Primer
gaatgaagtgttcctttcgggattgatcgtt

SEQ ID NO: 22 - DNA, 22 bp, Artificial Sequence
  Description of Artificial Sequence Primer
actcactgattgcgtttcctag

SEQ ID NO: 23 - DNA, 43 bp, Artificial Sequence
  Description of Artificial Sequence Primer
ttaattaacacgtggcgcgccagtcttcttcttcaaggtgacc

SEQ ID NO: 24 - DNA, 41 bp, Artificial Sequence
  Description of Artificial Sequence Primer
ggcgcgccacgtgttaattaactgattcgactaggcgcctc

SEQ ID NO: 25 - DNA, 31 bp, Artificial Sequence
  Description of Artificial Sequence Primer
ccttggcgcgccttcactctcttctccttcc

SEQ ID NO: 26 - DNA, 32 bp, Artificial Sequence
  Description of Artificial Sequence Primer
ccccttaattaatctggtcttgaagcttgtcc

SEQ ID NO: 27 - DNA, 31 bp, Artificial Sequence
  Description of Artificial Sequence Primer
ccttggcgcgcctctatgctctcttccgcta

SEQ ID NO: 28 - DNA, 33 bp, Artificial Sequence
  Description of Artificial Sequence Primer
ccccttaattaatccgatgatcctaatgaaggc

SEQ ID NO: 29 - DNA, 33 bp, Artificial Sequence
  Description of Artificial Sequence Primer
ccttggcgcgccatacggtatacgtttctacac

SEQ ID NO: 30 - DNA, 30 bp, Artificial Sequence
  Description of Artificial Sequence Primer
ccccttaattaaagacggcgtctatatccc

SEQ ID NO: 31 - DNA, 34 bp, Artificial Sequence
  Description of Artificial Sequence Primer
ggttggcgcgccaatgaagactaatctttttctc

SEQ ID NO: 32 - DNA, 33 bp, Artificial Sequence
  Description of Artificial Sequence Primer
ggggttaattaattagagttcgtcatgtttgta